(19) United States
(12) Patent Application Publication
(10) Pub. No.: US 2021/0315638 A1
(43) Pub. Date: Oct. 14, 2021

(54) SYSTEMS AND METHODS FOR IDENTIFYING AND CHARACTERIZING TISSUE AND PROVIDING TARGETED TREATMENT THEREOF

(71) Applicant: Neurent Medical Limited, Galway (IE)

(72) Inventors: David Townley, County Clare (IE); Swetha Rathan, Galway (IE); Cathal Mclaughlin, Galway (IE); Anup Poudel, Galway (IE); Manus Biggs, Galway (IE)

(21) Appl. No.: 17/225,603

(22) Filed: Apr. 8, 2021

Related U.S. Application Data
(60) Provisional application No. 63/007,639, filed on Apr. 9, 2020.

(51) Int. Cl. A61B 34/10; A61B 18/14; G16H 20/30; G06N 3/04; G06N 3/08

(52) U.S. Cl. CPC A61B 34/10; A61B 18/1492; A61B 2018/00267; G06N 3/04; G06N 3/08; G16H 20/30

(57) ABSTRACT

The invention generally relates to systems and methods for providing detection, identification, and precision targeting of specific tissue of interest to undergo a therapeutic treatment while minimizing or avoiding collateral damage to surrounding or adjacent non-targeted tissue.

### CROSS-REFERENCE TO RELATED APPLICATIONS

This application claims the benefit of, and priority to, U.S. Provisional Patent Application No. 63/007,639, filed Apr. 9, 2020, the contents of which are incorporated by reference.

### FIELD OF THE INVENTION

The invention generally relates to systems and methods for providing detection, identification, and precision targeting of specific tissue(s) of interest to undergo a therapeutic treatment while minimizing or avoiding collateral damage to surrounding or adjacent non-targeted tissue.

### BACKGROUND

Certain surgical procedures, such as ablation therapy, require a surgeon to apply precise treatment to the intended target site (i.e., tissue intended to receive treatment) at appropriate levels so as to avoid collateral damage to surrounding tissue, which can lead to further complications and even death. For example, certain procedures require increased precision due to the nature tissue to be treated and the location of such tissue in relation to any nearby or underlying tissue that may be highly sensitive and/or is critical to keep intact and free of unintended damage (i.e., blood vessels, nerves, etc.).

For example, many neuromodulation procedures require such precision. Neuromodulation refers to the alteration, or modulation, of nerve activity by delivering electrical (or sometimes pharmaceutical) agents directly to a target area. The delivery of electrical stimulation can result in partial or complete incapacitation, or other effective disruption, of neural activity. Therapeutic neuromodulation, for example, can include partially or completely inhibiting, reducing, and/or blocking neural communication along neural fibers for the treatment of certain conditions and disorders, specifically for pain relief and/or restoration of function. Some conditions and disorders that may be treated via neuromodulation include, but are not limited to, epilepsy, migraine headaches, spinal cord injuries, Parkinson's disease, and urinary incontinence, to name a few. Neuromodulation can also be used to treat certain conditions associated with the nose, such as rhinosinusitis, including, but not limited to, allergic rhinitis, non-allergic rhinitis, chronic rhinitis, acute rhinitis, recurrent rhinitis, chronic sinusitis, acute sinusitis, recurrent sinusitis, and medical resistant rhinitis and/or sinusitis, in addition to combinations of one or more of the preceding conditions.

Neuromodulation treatment procedures may generally involve the application of electrodes to the brain, the spinal cord, or peripheral nerves for subsequent treatment of conditions or disorders associated therewith. The electrodes are coupled, via an extension cable, to a pulse generator and power source, which generates the necessary electrical stimulation. An electrical current passes from the generator to the nerve, and can either inhibit pain signals or stimulate neural impulses where they were previously absent. Importantly, electrodes must be precisely placed and the level of electrical stimulation must be controlled so as to avoid or minimize creating collateral damage to surrounding or adjacent non-neural structures, such as bone and blood vessels, as well as non-targeted neural tissue.

Peripheral nerve stimulation is a commonly used approach to treat peripheral neurological conditions and conditions, including chronic pain. In order to establish accurate placement of electrodes and level of electrical stimulation to the targeted peripheral nerve, peripheral nerve stimulation treatment typically requires an initial testing or trial period. For example, a small electrical device (a wire-like electrode) is surgically implanted and placed next to one of the peripheral nerves. The electrode delivers rapid electrical pulses during the initial testing period (trial) to determine whether the electrical pulses result in the desired effect. Once the desired effect is established (via repositioning and/or adjusting of electrical stimulation levels) a more permanent electrode may be implanted into a patient's body. Accordingly, a drawback to current neuromodulation procedures, notably neuromodulation of peripheral nerves, is that such procedures cannot precisely target neural tissue, thereby presenting risk of causing significant collateral damage to surrounding non-neural tissue (such as blood vessels), and/or other non-targeted neural tissue.

Another exemplary procedure requiring precision includes interventional cardiac electrophysiology (EP) procedures, for example. In such a procedure, it is often necessary for the surgeon to determine the condition of cardiac tissue at a target ablation site in or near the heart. During some EP procedures, the surgeon may deliver a mapping catheter through a main vein or artery into an interior region of the heart to be treated. Using the mapping catheter, the surgeon may then determine the source of a cardiac rhythm disturbance or abnormality by placing a number of mapping elements carried by the catheter into contact with the adjacent cardiac tissue and then operating the catheter to generate an electrophysiology map of the interior region of the heart based on sensed electrical cardiac signals. Once a map of the heart is generated, the surgeon may then advance an ablation catheter into the heart, and position an ablation electrode carried by the catheter tip near the targeted cardiac tissue to ablate the tissue and form a lesion, thereby treating the cardiac rhythm disturbance or abnormality. In some techniques, the ablation catheter itself may include a number of mapping electrodes, allowing the same device to be used for both mapping and ablation.

Various ultrasound-based imaging catheters and probes have been developed for visualizing body tissue in applications such as interventional cardiology, interventional radiology, and electrophysiology. For interventional cardiac electrophysiology procedures, for example, ultrasound imaging devices have been developed that permit the visualization of anatomical structures of the heart directly and in real-time. While such imaging-based products allow some form of visualization of the targeted tissue, such procedures still lack the ability to precisely target and apply treatment to the tissue of interest while reducing or eliminating the risk of further treatment non-targeted, adjacent tissue.

### SUMMARY

The invention recognizes that knowing certain bioelectric properties of tissue, both active and passive, specifically interfacial polarization, dielectric dispersion, and dielectric relaxation phenomena/behavior of tissue, at a given target site prior to electrotherapeutic treatment (i.e., neuromodulation, ablation, etc.) provides an ability to more precisely target a specific tissue of interest (i.e., targeted tissue) and minimize and/or prevent collateral damage to adjacent or surrounding non-targeted tissue.

For example, certain target sites intended to undergo treatment may consist of more than one type of tissue (i.e., nerves, muscles, bone, blood vessels, etc.). In particular, a tissue of interest (i.e., the specific tissue to undergo treatment) may be adjacent to one or more tissues that are not of interest (i.e., tissue that is not intended to undergo treatment). In one scenario, a surgeon may wish to provide electrotherapeutic stimulation to a nerve tissue, while avoiding providing any such stimulation to an adjacent blood vessel, for example, as unintended collateral damage may result in damage to the blood vessel and cause further complications. In such a scenario, the specific type of targeted tissue may generally dictate the level of electrical stimulation required to elicit a desired effect. Furthermore, physical properties of the targeted tissue, including the specific location and depth of the targeted tissue, in relation to the non-targeted tissue, further impacts the level of electrical stimulation necessary to result in effective therapeutic treatment.

The invention provides systems and methods with an ability to characterize, prior to an electrotherapeutic treatment, tissue at a target site by sensing bioelectric properties of tissue, wherein such characterization includes identifying specific types of tissue present and further determining a interfacial polarization, or dielectric dispersion and relaxation phenomena/behavior pattern for the identified types of tissue. For example, different tissue types include different physiological and histological characteristics (e.g., cell components, proteins, etc.). As a result of the different characteristics, different tissue types have different associated bioelectrical properties and thus exhibit different associated electrical behavior in response to application of energy and frequencies applied thereto. One change in such electrical behavior is referred to as relaxation phenomena. The relaxation phenomena of a given tissue occurs at a particular electrical frequency in which the membranes of cells of the given tissue become permeable to thereby allow electrical stimulation current (at the particular frequency) to flow through the membrane to thereby elicit a desired effect upon the tissue. When a tissue is not exhibiting the relaxation phenomena (i.e., when electrical stimulation current is tuned to a different frequency that does not correlate to the relaxation phenomena), the membranes of cells of the given tissue may or may not be permeable to that specific electrical stimulation current and thus do not elicit an effect. The systems and methods are further configured to tune energy output (i.e., delivery of electrotherapeutic stimulation) based on the these relaxation patterns of a tissue of interest such that the energy delivered is at a specific frequency configured to target the tissue of interest while avoiding the non-targeted tissue (i.e., the energy is tuned to a frequency level associated with the dielectric relaxation phenomena of the targeted tissue only).

Accordingly, the invention solves the problem of causing unnecessary collateral damage to non-targeted tissue during a procedure involving the application of electrotherapeutic stimulation at a target site composed of a variety of tissue types. In particular, the systems and methods are able to, prior to treatment, characterize and identify tissue types and further identify specific levels of energy (i.e., specific target frequencies) to be delivered so as to cause only those intended, targeted tissues to exhibit dielectric relaxation phenomena and thus receive the energy for treatment, while non-targeted tissues remain intact and avoid collateral damage.

One aspect of the present invention provides a system for treating a condition. The system includes a device comprising an end effector including a plurality of electrodes and a controller operably associated with the device. The controller is configured to receive data from the device associated with bioelectric properties of one or more tissues at the target site and process the data to identify a type of each of the one or more tissues at the target site and further identify a dielectric relaxation pattern for each of the one or more identified tissue types. The controller is further configured to determine an ablation pattern to be delivered by one or more of the plurality of electrodes of the end effector based on identified dielectric relaxation patterns. The ablation energy associated with the ablation pattern is at a level sufficient to ablate a targeted tissue and minimize and/or prevent collateral damage to surrounding or adjacent non-targeted tissue at the target site.

The bioelectric properties may include, but are not limited to, complex impedance, resistance, reactance, capacitance, inductance, complex, real and imaginary dielectric permittivity, conductivity, dielectric properties, muscle or nerve firing voltage, muscle or nerve firing current, depolarization, hyperpolarization, magnetic field, induced electromotive force, and combinations thereof. The dielectric properties may include, for example, at least a complex dielectric permittivity. It should be noted that, in some embodiments, a subset of the plurality of electrodes is configured to deliver non-therapeutic stimulating energy at a frequency/waveform to respective positions at the target site to thereby sense the bioelectric properties of the one or more tissues at the target site.

The processing of the data may include, but is not limited to, comparing the data received from the device with electric signatures, as well as data with different dielectric models (e.g., Havriliak-Negami (HN) relaxation) to determine the dielectric parameters associated with a plurality of known tissue types. For example, the controller may be configured to compare the tissue data (i.e., data received from the treatment device associated with tissue at the target site) with profiles of known tissue types stored in a database, for example. Each profile may generally include electric signature data that generally characterizes a known tissue type, including physiological, histological, and bioelectric properties of a known tissue type, including dielectric relaxation phenomena/behavior of the tissue and the specific frequency value at which the tissue exhibits the dielectric relaxation phenomena/behavior.

In some embodiments, the ablation energy is tuned to a target frequency associated with a dielectric relaxation pattern of the targeted tissue. The target frequency comprises a frequency at which the targeted tissue exhibits relaxation phenomena behavior and the non-targeted tissue does not exhibit relaxation phenomena behavior. In particular, delivery of the ablation energy, tuned to the target frequency, penetrates a membrane of one or more cells associated only with the targeted tissue.

In some embodiments, the condition includes a peripheral neurological condition. The peripheral neurological condition may be associated with a nasal condition or a non-nasal condition of the patient. For example, the non-nasal condition may include atrial fibrillation (AF). In some embodiments, the nasal condition may include rhinosinusitis. Accordingly, in some embodiments, the target site is within a sino-nasal cavity of the patient. The delivery of the ablation energy may result in disruption of multiple neural signals to, and/or result in local hypoxia of, mucus producing and/or mucosal engorgement elements within the sino-nasal cavity of the patient. The targeted tissue is proximate or inferior to a sphenopalatine foramen. Yet still, delivery of the ablation energy may result in therapeutic modulation of postganglionic parasympathetic nerves innervating nasal mucosa at foramina and or microforamina of a palatine bone of the patient. In particular, delivery of the ablation energy causes multiple points of interruption of neural branches extending through foramina and microforamina of palatine bone. Yet still, in some embodiments, delivery of the ablation energy may cause thrombus formation within one or more blood vessels associated with mucus producing and/or mucosal engorgement elements within the nose. The resulting local hypoxia of the mucus producing and/or mucosal engorgement elements may result in decreased mucosal engorgement to thereby increase volumetric flow through a nasal passage of the patient. Additionally, or alternatively, the resulting local hypoxia may cause neuronal degeneration.

Another aspect of the invention provides a method for treating a condition. The method includes providing a device comprising an end effector including a plurality of electrodes and a controller operably associated with the device. The method further includes positioning the end effector at a target site associated with a patient and receiving, via the controller, data from the device associated with bioelectric properties of one or more tissues at the target site. The method further includes processing, via the controller, the data to identify a type of each of the one or more tissues at the target site and further identify a dielectric relaxation pattern for each of the one or more identified tissue types. The method further includes determining, via the controller, an ablation pattern to be delivered by one or more of the plurality of electrodes of the end effector based on identified dielectric relaxation patterns. The ablation energy associated with the ablation pattern is at a level sufficient to ablate a targeted tissue and minimize and/or prevent collateral damage to surrounding or adjacent non-targeted tissue at the target site.

The bioelectric properties may include, but are not limited to, complex impedance, resistance, reactance, capacitance, inductance, complex, real and imaginary dielectric permittivity, conductivity, dielectric properties, muscle or nerve firing voltage, muscle or nerve firing current, depolarization, hyperpolarization, magnetic field, induced electromotive force, and combinations thereof. The dielectric properties may include, for example, at least a dielectric modulus or a complex dielectric permittivity. It should be noted that, in some embodiments, a subset of the plurality of electrodes is configured to deliver non-therapeutic stimulating energy at a frequency/waveform to respective positions at the target site to thereby sense the bioelectric properties of the one or more tissues at the target site.

The processing of the data may include, but is not limited to, comparing the data received from the device with electric signature, and training of the data with different dielectric models (e.g., Havriliak-Negami (HN) relaxation) to determine the dielectric parameters associated with a plurality of known tissue types. For example, the controller may be configured to compare the tissue data (i.e., data received from the treatment device associated with tissue at the target site) with profiles of known tissue types stored in a database, for example. Each profile may generally include electric signature data that generally characterizes a known tissue type, including physiological, histological, and bioelectric properties of a known tissue type, including dielectric relaxation phenomena/behavior of the tissue and the specific frequency value at which the tissue exhibits the dielectric relaxation phenomena/behavior.

In some embodiments, the ablation energy is tuned to a target frequency associated with a dielectric relaxation pattern of the targeted tissue. The target frequency comprises a frequency at which the targeted tissue exhibits relaxation phenomena behavior and the non-targeted tissue does not exhibit relaxation phenomena behavior. In particular, delivery of the ablation energy, tuned to the target frequency, penetrates a membrane of one or more cells associated only with the targeted tissue.

In some embodiments, the condition includes a peripheral neurological condition. The peripheral neurological condition may be associated with a nasal condition or a non-nasal condition of the patient. For example, the non-nasal condition may include atrial fibrillation (AF). In some embodiments, the nasal condition may include rhinosinusitis. Accordingly, in some embodiments, the target site is within a sino-nasal cavity of the patient. The delivery of the ablation energy may result in disruption of multiple neural signals to, and/or result in local hypoxia of, mucus producing and/or mucosal engorgement elements within the sino-nasal cavity of the patient. The targeted tissue is proximate or inferior to a sphenopalatine foramen. Yet still, delivery of the ablation energy may result in therapeutic modulation of postganglionic parasympathetic nerves innervating nasal mucosa at foramina and or microforamina of a palatine bone of the patient. In particular, delivery of the ablation energy causes multiple points of interruption of neural branches extending through foramina and microforamina of palatine bone. Yet still, in some embodiments, delivery of the ablation energy may cause thrombus formation within one or more blood vessels associated with mucus producing and/or mucosal engorgement elements within the nose. The resulting local hypoxia of the mucus producing and/or mucosal engorgement elements may result in decreased mucosal engorgement to thereby increase volumetric flow through a nasal passage of the patient. Additionally, or alternatively, the resulting local hypoxia may cause neuronal degeneration.

### BRIEF DESCRIPTION OF THE DRAWINGS

FIGS. 1A and 1B are diagrammatic illustrations of a system for treating a condition of a patient using a handheld device according to some embodiments of the present disclosure.

FIG. 2 is a diagrammatic illustration of the console coupled to the handheld device consistent with the present disclosure, further illustrating one embodiment of an end effector of the handheld device for delivering energy to tissue at one or more target sites.

FIG. 3 is a side view of one embodiment of a handheld device for providing therapeutic treatment consistent with the present disclosure.

FIG. 4 is an enlarged, perspective view of one embodiment of an end effector consistent with the present disclosure.

FIGS. 5A-5F are various views of the multi-segment end effector consistent with the present disclosure.

FIG. 5A is an enlarged, perspective view of the multi-segment end effector illustrating the first (proximal) segment and second (distal) segment. FIG. 5B is an exploded, perspective view of the multi-segment end effector. FIG. 5C is an enlarged, top view of the multi-segment end effector. FIG. 5D is an enlarged, side view of the multi-segment end effector. FIG. 5E is an enlarged, front (proximal facing) view of the first (proximal) segment of the multi-segment end effector. FIG. 5F is an enlarged, front (proximal facing) view of the second (distal) segment of the multi-segment end effector.

FIG. 6 is a perspective view, partly in section, of a portion of a support element illustrating an exposed conductive wire serving as an energy delivery element or electrode element.

FIG. 7 is a cross-sectional view of a portion of the shaft of the handheld device taken along lines 7-7 of FIG. 3.

FIG. 8A is a side view of the handle of the handheld device.

FIG. 8B is a side view of the handle illustrating internal components enclosed within.

FIG. 9A is a block diagram illustrating delivery of non-therapeutic energy from electrodes of the end effector at a frequency/waveform for sensing one or more properties associated with one or more tissues at a target site in response to the non-therapeutic energy.

FIG. 9B is a block diagram illustrating communication of sensor data from the handheld device to the controller and subsequent tuning, via the controller, of energy output based on the sensor data for precision targeting of tissue of interest and to be treated.

FIG. 9C is a block diagram illustrating delivery of energy to the target site tuned to a specific frequency to elicit dielectric relaxation phenomena/behavior in the targeted tissue (based on the ablation pattern output from the controller).

FIG. 10 is a block diagram illustrating delivery of energy to the target site, and specifically illustrating flow of current through membranes of cells of the targeted tissue (which is exhibit dielectric relaxation phenomena/behavior) and flow of current around membranes of cells of non-targeted tissue (which is not exhibiting dielectric relaxation phenomena/behavior) as a result of the energy being tuned to a target frequency.

FIG. 11 is a flow diagram illustrating one embodiment of a method for treating a condition.

FIG. 12 is a schematic of an exemplary probe/electrode setup for performing some of the methods described herein, most notably for characterizing tissue at a target site by sensing bioelectric properties of tissue, wherein such characterization includes identifying specific types of tissue present and further determining a dielectric relaxation phenomena/behavior pattern for the identified types of tissue. FIG. 12A is a schematic diagram of one embodiment of a 3-probe/electrode system for sensing bioelectric properties of tissue for subsequent characterization of tissue at a target site, wherein such characterization includes identifying specific types of tissue present and further determining a dielectric relaxation phenomena/behavior pattern for the identified types of tissue.

FIGS. 13A and 13B are graphs illustrating dielectric properties of two tissue types (spinal cord and muscle tissues), including the plotting of loss tangent value relative to frequency (FIG. 13A) and the plotting of an imaginary electric modulus relative to frequency (FIG. 13B).

FIGS. 14A-14H are graphs illustrating the plotting of real and imaginary values of the complex relative permittivity (based on the Havriliak-Negami (HN) relaxation phenomena model) relative to frequency for the two tissue types of FIGS. 13A and 13B (spinal cord and muscle tissues).

FIGS. 14A and 14B illustrate plotting of real and imaginary values of the complex relative permittivity relative to frequency for upper spinal cord tissue.

FIGS. 14C and 14D illustrate plotting of real and imaginary values of the complex relative permittivity relative to frequency for lower spinal cord tissue.

FIGS. 14E and 14F are graphs illustrating the plotting of real and imaginary values of the complex relative permittivity relative to frequency for lower back muscle tissue.

FIGS. 14G and 14H are graphs illustrating the plotting of real and imaginary values of the complex relative permittivity relative to frequency for upper back muscle tissue.

FIGS. 15A and 15B are graphs illustrating dielectric properties of different portions of a tissue (turbinate tissue), including the plotting of loss tangent value relative to frequency (FIG. 15A) and the plotting of an imaginary electric modulus relative to frequency (FIG. 15B).

FIGS. 16A-16F are graphs illustrating the plotting of real and imaginary values of the complex relative permittivity (based on the HN relaxation phenomena model) relative to frequency for the different portions of the turbinate tissue of FIGS. 15A and 15B.

FIGS. 16A and 16B illustrate plotting of real and imaginary values of the complex relative permittivity relative to frequency for the end of a turbinate tissue.

FIGS. 16C and 16D illustrate plotting of real and imaginary values of the complex relative permittivity relative to frequency for the center of the turbinate tissue.

FIGS. 16E and 16F are graphs illustrating the plotting of real and imaginary values of the complex relative permittivity relative to frequency for portions of the turbinate tissue near blood vessels.

### DETAILED DESCRIPTION

The invention recognizes that knowing certain bioelectric properties of tissue, both active and passive, specifically interfacial polarization, dielectric dispersion, and dielectric relaxation phenomena/behavior of tissue, at a given target site prior to electrotherapeutic treatment (i.e., neuromodulation, ablation, etc.) provides an ability to more precisely target a specific tissue of interest (i.e., targeted tissue) and minimize and/or prevent collateral damage to adjacent or surrounding non-targeted tissue.

For example, certain target sites intended to undergo treatment may consist of more than one type of tissue (i.e., nerves, muscles, bone, blood vessels, etc.). In particular, a tissue of interest (i.e., the specific tissue to undergo treatment) may be adjacent to one or more tissues that are not of interest (i.e., tissue that is not intended to undergo treatment). In one scenario, a surgeon may wish to provide electrotherapeutic stimulation to a nerve tissue, while avoiding providing any such stimulation to an adjacent blood vessel, for example, as unintended collateral damage may result in damage to the blood vessel and cause further complications. In such a scenario, the specific type of targeted tissue may generally dictate the level of electrical stimulation required to elicit a desired effect. Furthermore, physical properties of the targeted tissue, including the specific location and depth of the targeted tissue, in relation to the non-targeted tissue, further impacts the level of electrical stimulation necessary to result in effective therapeutic treatment.

Neuromodulation, for example, is technology that acts directly upon nerves. It is the alteration, or modulation, of nerve activity by delivering electrical or pharmaceutical agents directly to a target area. Neuromodulation devices and treatments have been shown to be highly effective at treating a variety of conditions and disorders. The most common indication for neuromodulation is treatment of chronic pain. However, the number of neuromodulation applications over the years has increased to include more than just the treatment of chronic pain, such as deep brain stimulation (DBS) treatment for Parkinson's disease, sacral nerve stimulation for pelvic disorders and incontinence, and spinal cord stimulation for ischemic disorders (angina, peripheral vascular disease).

Neuromodulation is particularly useful in the treatment of peripheral neurological disorders. There are currently over 100 kinds of peripheral nerve disorders, which can affect one nerve or many nerves. Some are the result of other diseases, like diabetic nerve problems. Others, like Guillain-Barre syndrome, happen after a virus infection. Still others are from nerve compression, like carpal tunnel syndrome or thoracic outlet syndrome. In some cases, like complex regional pain syndrome and brachial plexus injuries, the problem begins after an injury. However, some people are born with peripheral neurological disorders.

Peripheral nerve stimulation has become established for very specific clinical indications, including certain complex regional pain syndromes, pain due to peripheral nerve injuries, and the like. Some of the common applications of peripheral nerve stimulation include treatment of back pain, occipital nerve stimulation for treatment of migraine headaches, and pudendal nerve stimulation that is being investigated for use in urinary bladder incontinence.

The invention provides systems and methods with an ability to characterize, prior to an electrotherapeutic treatment such as neuromodulation, tissue at a target site by sensing bioelectric properties of tissue, wherein such characterization includes identifying specific types of tissue present and further determining a dielectric relaxation phenomena/behavior pattern for the identified types of tissue. For example, different tissue types include different physiological and histological characteristics (e.g., cell components, extracellular proteins, etc.). As a result of the different characteristics, different tissue types have different associated electrical and electrochemical properties and thus exhibit different associated behavior in response to application of energy and/or frequency applied thereto. The electrical behavior of tissue type (capacitive to resistive or vice versa) alters at specific frequencies due to the relaxation phenomena. The interfacial polarization , dielectric dispersion and relaxation phenomenon of a given tissue occurs at a particular electrical frequency in which the membranes of cells of the given tissue become permeable to thereby allow electrical stimulation current (at the particular frequency) to flow through the membrane to thereby elicit a desired effect upon the tissue.

For example, the alternating current (AC) energy transfer through the tissue type takes place either through capacitive or resistive means and is highly dependent on the frequency of an energy used. For example, if the transfer of energy at specific frequency in tissue type taking place though relatively higher resistive means, these phenomena will slowly alter upon changing frequency and, at certain frequency, the conduction though capacitive behavior becomes active. These phenomena are generally represented Maxwell-Wagner-Sillar (MWS) relaxation. Similarly, the permeability of current types (direct current or alternating current) depends on the specific frequency and varies with the cell types, constituency and morphology. The dielectric permittivity and dielectric relaxation frequency heighten when a tissue is stimulated at specific frequency. Below the specific relaxation frequency such as dielectric relaxation, the tissue is highly permeable to the alternating current. However, at the region of relaxation frequency, the heating effects become dominant. Hence, when a tissue is not exhibiting the dielectric relaxation phenomena (i.e., when electrical stimulation current is tuned to a different frequency (i.e., below and above the relaxation frequency) that does not correlate to the dielectric relaxation phenomena), the membranes of cells of the given tissue are not permeable to that specific electrical stimulation current and thus do not elicit an effect. The systems and methods are further configured to tune energy output (i.e., delivery of electrotherapeutic stimulation) based on the dielectric relaxation pattern of a tissue of interest such that the energy delivered is at a specific frequency configured to target the tissue of interest while avoiding the non-targeted tissue (i.e., the energy is tuned to a frequency level associated with the dielectric relaxation phenomena of the targeted tissue only).

Accordingly, the invention solves the problem of causing unnecessary collateral damage to non-targeted tissue during a procedure involving the application of electrotherapeutic stimulation at a target site composed of a variety of tissue types. In particular, the systems and methods are able to, prior to treatment, characterize and identify tissue types and further identify specific levels of energy (i.e., specific target frequencies) to be delivered so as to cause only those intended, targeted tissues to exhibit dielectric relaxation phenomena and thus receive the energy for treatment, while non-targeted tissues remain intact and avoid collateral damage.

It should be noted that, although many of the embodiments are described with respect to devices, systems, and methods for therapeutically modulating nerves associated with the peripheral nervous system (PNS) and thus the treatment of peripheral neurological conditions or disorders, other applications and other embodiments in addition to those described herein are within the scope of the present disclosure. For example, at least some embodiments of the present disclosure may be useful for the treatment of other disorders, such as the treatment of disorders associated with the central nervous system.

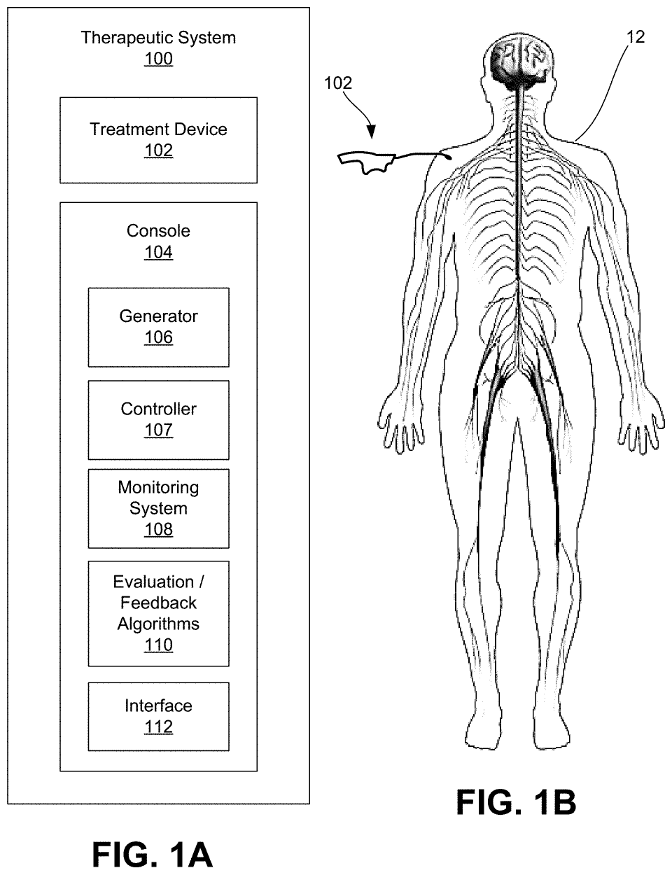

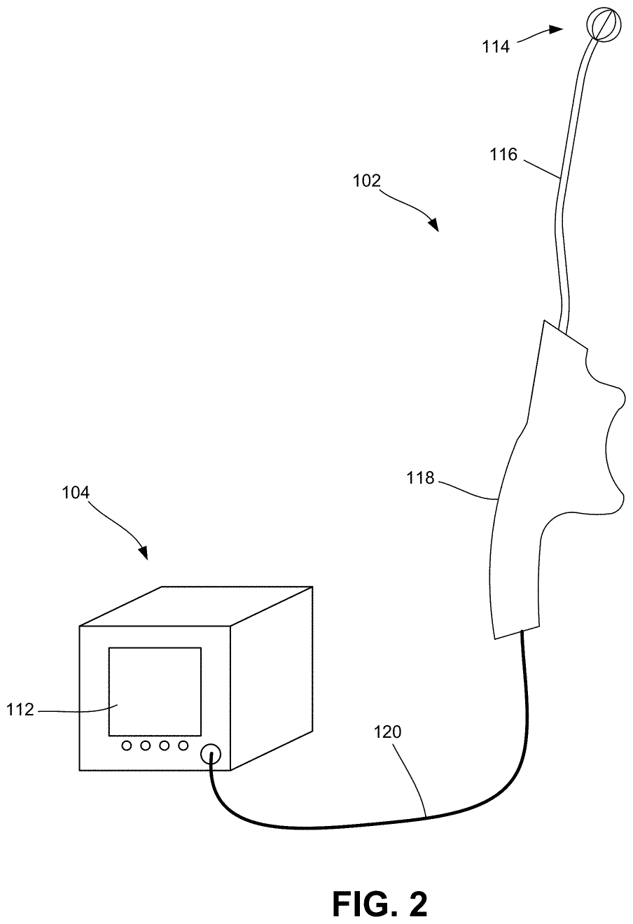

FIGS. 1A and 1B are diagrammatic illustrations of a therapeutic system 100 for treating a condition of a patient using a handheld device 102 according to some embodiments of the present disclosure. The system 100 generally includes a device 102 and a console 104 to which the device 102 is to be connected. FIG. 2 is a diagrammatic illustrations of the console 104 coupled to the handheld device 102 illustrating an exemplary embodiment of an end effector 114 for delivering energy to tissue at the one or more target sites of a patient for the treatment of a neurological disorder. As illustrated, the device 102 is a handheld device, which includes end effector 114, a shaft 116 operably associated with the end effector 114, and a handle 118 operably associated with the shaft 116. The end effector 114 may be collapsible/retractable and expandable, thereby allowing for the end effector 114 to be minimally invasive (i.e., in a collapsed or retracted state) upon delivery to one or more target sites within a patient and then expanded once positioned at the target site. It should be noted that the terms “end effector” and “therapeutic assembly” may be used interchangeably throughout this disclosure.

For example, a surgeon or other medical professional performing a procedure can utilize the handle 118 to manipulate and advance the shaft 116 to a desired target site, wherein the shaft 116 is configured to locate at least a distal portion thereof intraluminally at a treatment or target site within a portion of the patient associated with tissue to undergo electrotherapeutic stimulation for subsequent treatment of an associated condition or disorder. In the event that the tissue to be treated is a nerve, such that electrotherapeutic stimulation thereof results in treatment of an associated neurological condition, the target site may generally be associated with peripheral nerve fibers. The target site may be a region, volume, or area in which the target nerves are located and may differ in size and shape depending upon the anatomy of the patient. Once positioned, the end effector 114 may be deployed and subsequently deliver energy to the one or more target sites. The energy delivered may be non-therapeutic stimulating energy at a frequency for locating neural tissue and further sensing one or more properties of the neural tissue. For example, the end effector 114 may include an electrode array, which includes at least a subset of electrodes configured to sense the presence of neural tissue at a respective position of each of the electrodes, as well as morphology of the neural tissue, wherein such data may be used for determining, via the console 104, the type of neural tissue as well as a dielectric relaxation phenomena/behavior pattern for the identified neural tissue.

Based on the identification of the neural tissue type and a dielectric relaxation phenomena/behavior pattern of the neural tissue, the console 104 is configured to tune energy output (i.e., delivery of electrotherapeutic stimulation) based on the dielectric relaxation pattern of the targeted tissue such that the energy delivered from the end effector 114 upon the target site is at a specific frequency so as to therapeutically modulate the neural tissue (i.e., the energy is tuned to a frequency level associated with the dielectric relaxation phenomena of the targeted tissue only) and minimize and/or prevent damage to non-target neural tissue and/or non-target anatomical structures at the target site, such as blood vessels and/or bone. Accordingly, the end effector 114 is able to therapeutically modulating nerves of interest, particularly nerves associated with a peripheral neurological conditional or disorder so as to treat such condition or disorder, while minimizing and/or preventing collateral damage.

For example, the end effector 114 may include at least one energy delivery element, such as an electrode, configured to delivery energy to the target tissue which may be used for sensing presence and/or specific properties of tissue (such tissue including, but not limited to, muscle, nerves, blood vessels, bones, etc.) for therapeutically modulating tissues of interest, such as neural tissue. For example, one or more electrodes may be provided by one or more portions of the end effector 114, wherein the electrodes may be configured to apply electromagnetic neuromodulation energy (e.g., radiofrequency (RF) energy) to target sites. In other embodiments, the end effector 114 may include other energy delivery elements configured to provide therapeutic neuromodulation using various other modalities, such as cryotherapeutic cooling, ultrasound energy (e.g., high intensity focused ultrasound (“HIFU”) energy), microwave energy (e.g., via a microwave antenna), direct heating, high and/or low power laser energy, mechanical vibration, and/or optical power.

In some embodiments, the end effector 114 may include one or more sensors (not shown), such as one or more temperature sensors (e.g., thermocouples, thermistors, etc.), impedance sensors, and/or other sensors. The sensors and/or the electrodes may be connected to one or more wires extending through the shaft 116 and configured to transmit signals to and from the sensors and/or convey energy to the electrodes.

As shown, the device 102 is operatively coupled to the console 104 via a wired connection, such as cable 120. It should be noted, however, that the device 102 and console 104 may be operatively coupled to one another via a wireless connection. The console 104 is configured to provide various functions for the device 102, which may include, but is not limited to, controlling, monitoring, supplying, and/or otherwise supporting operation of the device 102. For example, when the device 102 is configured for electrode-based, heat-element-based, and/or transducer-based treatment, the console 104 may include an energy generator 106 configured to generate RF energy (e.g., monopolar, bipolar, or multi-polar RF energy), pulsed electrical energy, microwave energy, optical energy, ultrasound energy (e.g., intraluminally-delivered ultrasound and/or HIFU), direct heat energy, radiation (e.g., infrared, visible, and/or gamma radiation), and/or another suitable type of energy.

In some embodiments, the console 104 may include a controller 107 communicatively coupled to the device 102. However, in the embodiments described herein, the controller 107 may generally be carried by and provided within the handle 118 of the device 102. The controller 107 is configured to initiate, terminate, and/or adjust operation of one or more electrodes provided by the end effector 114 directly and/or via the console 104. For example, the controller 107 can be configured to execute an automated control algorithm and/or to receive control instructions from an operator (e.g., surgeon or other medical professional or clinician). For example, the controller 107 and/or other components of the console 104 (e.g., processors, memory, etc.) can include a computer-readable medium carrying instructions, which when executed by the controller 107, causes the device 102 to perform certain functions (e.g., apply energy in a specific manner, detect impedance, detect temperature, detect nerve locations or anatomical structures, etc.). A memory includes one or more of various hardware devices for volatile and non-volatile storage, and can include both read-only and writable memory. For example, a memory can comprise random access memory (RAM), CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory.

The console 104 may further be configured to provide feedback to an operator before, during, and/or after a treatment procedure via evaluation/feedback algorithms 110. For example, the evaluation/feedback algorithms 110 can be configured to provide information associated with the location of nerves at the treatment site, the temperature of the tissue at the treatment site, and/or the effect of the therapeutic neuromodulation on the nerves at the treatment site. In certain embodiments, the evaluation/feedback algorithm 110 can include features to confirm efficacy of the treatment and/or enhance the desired performance of the system 100. For example, the evaluation/feedback algorithm 110, in conjunction with the controller 107, can be configured to monitor temperature at the treatment site during therapy and automatically shut off the energy delivery when the temperature reaches a predetermined maximum (e.g., when applying RF energy) or predetermined minimum (e.g., when applying cryotherapy). In other embodiments, the evaluation/feedback algorithm 110, in conjunction with the controller 107, can be configured to automatically terminate treatment after a predetermined maximum time, a predetermined maximum impedance rise of the targeted tissue (i.e., in comparison to a baseline impedance measurement), a predetermined maximum impedance of the targeted tissue), and/or other threshold values for biomarkers associated with autonomic function. This and other information associated with the operation of the system 100 can be communicated to the operator via a graphical user interface (GUI) 112 provided via a display on the console 104 and/or a separate display (not shown) communicatively coupled to the console 104, such as a tablet or monitor. The GUI 112 may generally provide operational instructions for the procedure, such as indicating when the device 102 is primed and ready to perform the treatment, and further providing status of therapy during the procedure, including indicating when the treatment is complete.

For example, as previously described, the end effector 114 and/or other portions of the system 100 can be configured to detect various parameters of a tissue of interest at the target site to determine the anatomy at the target site (e.g., tissue types, tissue locations, vasculature, bone structures, foramen, sinuses, etc.), locate nerves and/or other structures, and allow for neural mapping. For example, the end effector 114 may be configured to detect impedance, dielectric properties, temperature, and/or other properties that indicate the presence of neural tissue or fibers in the target region, as described in greater detail herein.

As shown in FIG. 1A, the console 104 further includes a monitoring system 108 configured to receive data from the end effector 114 (i.e., detected electrical and/or thermal measurements of tissue at the target site), specifically sensed by appropriate sensors (e.g., temperature sensors and/or impedance sensors, or the like), and process this information to identify the presence of nerves, the location of nerves, neural activity at the target site, and/or other properties of the neural tissue, such a physiological properties (e.g., depth), bioelectric properties, and thermal properties. The nerve monitoring system 108 can be operably coupled to the electrodes and/or other features of the end effector 114 via signal wires (e.g., copper wires) that extend through the cable 120 and through the length of the shaft 116. In other embodiments, the end effector 114 can be communicatively coupled to the nerve monitoring system 108 using other suitable communication means.

The nerve monitoring system 108 can determine neural locations and activity before therapeutic neuromodulation to determine precise treatment regions corresponding to the positions of the desired nerves. The nerve monitoring system 108 can further be used during treatment to determine the effect of the therapeutic neuromodulation, and/or after treatment to evaluate whether the therapeutic neuromodulation treated the target nerves to a desired degree. This information can be used to make various determinations related to the nerves proximate to the target site, such as whether the target site is suitable for neuromodulation. In addition, the nerve monitoring system 108 can also compare the detected neural locations and/or activity before and after therapeutic neuromodulation, and compare the change in neural activity to a predetermined threshold to assess whether the application of therapeutic neuromodulation was effective across the treatment site. For example, the nerve monitoring system 108 can further determine electroneurogram (ENG) signals based on recordings of electrical activity of neurons taken by the end effector 114 before and after therapeutic neuromodulation. Statistically meaningful (e.g., measurable or noticeable) decreases in the ENG signal(s) taken after neuromodulation can serve as an indicator that the nerves were sufficiently ablated. Additional features and functions of the nerve monitoring system 108, as well as other functions of the various components of the console 104, including the evaluation/feedback algorithms 110 for providing real-time feedback capabilities for ensuring optimal therapy for a given treatment is administered, are described in at least U.S. Publication No. 2016/0331459 and U.S. Publication No. 2018/0133460, the contents of each of which are incorporated by reference herein in their entireties.

The device 102 provides access to target sites associated with peripheral nerves for the subsequent neuromodulation of such nerves and treatment of a corresponding peripheral neurological condition or disorder. The peripheral nervous system is one of two components that make up the nervous system of bilateral animals, with the other part being the central nervous system (CNS). The PNS consists of the nerves and ganglia outside the brain and spinal cord. The main function of the PNS is to connect the CNS to the limbs and organs, essentially serving as a relay between the brain and spinal cord and the rest of the body. The peripheral nervous system is divided into the somatic nervous system and the autonomic nervous system. In the somatic nervous system, the cranial nerves are part of the PNS with the exception of the optic nerve (cranial nerve II), along with the retina. The second cranial nerve is not a true peripheral nerve but a tract of the diencephalon. Cranial nerve ganglia originated in the CNS. However, the remaining ten cranial nerve axons extend beyond the brain and are therefore considered part of the PNS. The autonomic nervous system exerts involuntary control over smooth muscle and glands. The connection between CNS and organs allows the system to be in two different functional states: sympathetic and parasympathetic. Accordingly, the devices, systems, and methods of the present invention are useful in detecting, identifying, and precision targeting nerves associated with the peripheral nervous system for treatment of corresponding peripheral neurological conditions or disorders.

The peripheral neurological conditions or disorders may include, but are not limited to, chronic pain, movement disorders, epilepsy, psychiatric disorders, cardiovascular disorders, gastrointestinal disorders, genitourinary disorders, to name a few. For example, chronic pain may include headaches, complex regional pain syndrome, neuropathy, peripheral neuralgia, ischemic pain, failed back surgery syndrome, and trigeminal neuralgia. The movement disorders may include spasticity, Parkinson's disease, tremor, dystonia, Tourette syndrome, camptocormia, hemifacial spasm, and Meige syndrome. The psychiatric disorders may include depression, obsessive compulsive disorder, drug addiction, and anorexia/eating disorders. The functional restoration may include restoration of certain functions post traumatic brain injury, hearing impairment, and blindness. The cardiovascular disorders may include angina, heart failure, hypertension, peripheral vascular disorders, and stroke. The gastrointestinal disorders may include dysmotility and obesity. The genitourinary disorders may include painful bladder syndrome, interstitial cystitis, and voiding dysfunction.

For example, the system 100 may be used for the treatment of a cardiovascular disorder, such as arrhythmias or heart rhythm disorders, including, but not limited to, atrial fibrillation (AF or A-fib). Atrial fibrillation is an irregular and often rapid heart rate that can increase one's risk of stroke, heart failure, and other heart-related complications. Atrial fibrillation occurs when regions of cardiac tissue abnormally conduct electric signals to adjacent tissue, thereby disrupting the normal cardiac cycle and causing asynchronous rhythm. Atrial fibrillation symptoms often include heart palpitations, shortness of breath, and weakness. While episodes of atrial fibrillation can come and go, a person may develop atrial fibrillation that doesn't go away and thus will require treatment. Although atrial fibrillation itself usually isn't life-threatening, it is a serious medical condition that sometimes requires emergency treatment, as it may lead to complications. For example, atrial fibrillation is associated with an increased risk of heart failure, dementia, and stroke.

The normal electrical conduction system of the heart allows the impulse that is generated by the sinoatrial node (SA node) of the heart to be propagated to and stimulate the myocardium (muscular layer of the heart). When the myocardium is stimulated, it contracts. It is the ordered stimulation of the myocardium that allows efficient contraction of the heart, thereby allowing blood to be pumped to the body. In AF, the normal regular electrical impulses generated by the sinoatrial node in the right atrium of the heart are overwhelmed by disorganized electrical impulses usually originating in the roots of the pulmonary veins. This leads to irregular conduction of ventricular impulses that generate the heartbeat. In particular, during AF, the heart's two upper chambers (the atria) beat chaotically and irregularly, out of coordination with the two lower chambers (the ventricles) of the heart.

During atrial fibrillation, the regular impulses produced by the sinus node for a normal heartbeat are overwhelmed by rapid electrical discharges produced in the atria and adjacent parts of the pulmonary veins. Sources of these disturbances are either automatic foci, often localized at one of the pulmonary veins, or a small number of localized sources in the form of either a re-entrant leading circle, or electrical spiral waves (rotors). These localized sources may be found in the left atrium near the pulmonary veins or in a variety of other locations through both the left or right atrium. There are three fundamental components that favor the establishment of a leading circle or a rotor: 1) slow conduction velocity of cardiac action potential; 2) short refractory period; and 3) small wavelength. Wavelength is the product of velocity and refractory period. If the action potential has fast conduction, with a long refractory period and/or conduction pathway shorter than the wavelength, an AF focus would not be established. In multiple wavelet theory, a wavefront will break into smaller daughter wavelets when encountering an obstacle, through a process called vortex shedding; but under proper conditions, such wavelets can reform and spin around a center, forming an AF focus.

The system 100 provides for the treatment of AF, in which the device 102 may provide access to and provide treatment of one or more target sites associated with nerves that correspond to, or are otherwise associated with, treating AF. For example, the device 102, in conjunction with the console 104, may detect, identify, and precision target cardiac tissue and subsequently deliver energy at a level or frequency sufficient to therapeutically modulate nerves associated with such cardiac tissue. The therapeutic modulation of such nerves is sufficient to disrupt the origin of the signals causing the AF and/or disrupt the conducting pathway for such signals.

Similar to the conduction system of the heart, a neural network exists which surrounds the heart and plays an important role in formation of the substrate of AF and when a trigger is originated, usually from pulmonary vein sleeves, AF occurs. This neural network includes ganglionated plexi (GP) located adjacent to pulmonary vein ostia which are under control of higher centers in normal people. For example, the heart is richly innervated by the autonomic nerves. The ganglion cells of the autonomic nerves are located either outside the heart (extrinsic) or inside the heart (intrinsic). Both extrinsic and intrinsic nervous systems are important for cardiac function and arrhythmogenesis. The vagal nerves include axons that come from various nuclei in the medulla. The extrinsic sympathetic nerves come from the paravertebral ganglia, including the superior cervical ganglion, middle cervical ganglion, the cervicothoracic (stellate) ganglion and the thoracic ganglia. The intrinsic cardiac nerves are found mostly in the atria, and are intimately involved in atrial arrhythmogenesis cardiovascular disorder, such as arrhythmias or heart rhythm disorders, including, but not limited to, atrial fibrillation. When GP become hyperactive owing to loss of inhibition from higher centers (e.g., in elderly), AF can occur.

The system 100 can be used to control hyperactive GP either by stimulating higher centers and their connections, such as vagus nerve stimulation, or simply by ablating GP. Accordingly, the device 102, in conjunction with the console 104, may detect and identify ganglionated plexus (GP) and further determine an energy level sufficient to therapeutically modulate or treat (i.e., ablate) the GP for the treatment of AF (i.e., surgically disrupting the origin of the signals causing the AF and disrupting the conducting pathway for such signals) while minimizing and/or preventing collateral damage to surrounding or adjacent non-neural tissue including bloods vessels and bone and non-targeted neural tissue. It should be noted that other nerves and/or cardiac tissue, or other structures, known to have an impact on or cause AF, may be targeted by the system 100, including, but not limited to, pulmonary veins (e.g., pulmonary vein isolation upon creation of lesions around PV ostia to prevent triggers from reaching atrial substrate).

In addition to treating arrhythmias, the system 100 may also be used for the treatment of other cardiovascular-related conditions, particularly those involving the kidney. The kidneys play a significant role in the progression of CHF, as well as in Chronic Renal Failure (CRF), End-Stage Renal Disease (ESRD), hypertension (pathologically high blood pressure), and other cardio-renal diseases.

The functions of the kidney can be summarized under three broad categories: filtering blood and excreting waste products generated by the body's metabolism; regulating salt, water, electrolyte and acid-base balance; and secreting hormones to maintain vital organ blood flow. Without properly functioning kidneys, a patient will suffer water retention, reduced urine flow and an accumulation of waste toxins in the blood and body. These conditions resulting from reduced renal function or renal failure (kidney failure) are believed to increase the workload of the heart.

For example, in a CHF patient, renal failure will cause the heart to further deteriorate as the water build-up and blood toxins accumulate due to the poorly functioning kidneys and, in turn, cause the heart further harm. CHF is a condition that occurs when the heart becomes damaged and reduces blood flow to the organs of the body. If blood flow decreases sufficiently, kidney function becomes impaired and results in fluid retention, abnormal hormone secretions and increased constriction of blood vessels. These results increase the workload of the heart and further decrease the capacity of the heart to pump blood through the kidney and circulatory system. This reduced capacity further reduces blood flow to the kidney. It is believed that progressively decreasing perfusion of the kidney is a principal non-cardiac cause perpetuating the downward spiral of CHF. Moreover, the fluid overload and associated clinical symptoms resulting from these physiologic changes are predominant causes for excessive hospital admissions, reduced quality of life, and overwhelming costs to the health care system due to CHF.

End-stage renal disease is another condition at least partially controlled by renal neural activity. There has been a dramatic increase in patients with ESRD due to diabetic nephropathy, chronic glomerulonephritis and uncontrolled hypertension. Chronic renal failure (CRF) slowly progresses to ESRD. CRF represents a critical period in the evolution of ESRD. The signs and symptoms of CRF are initially minor, but over the course of 2-5 years, become progressive and irreversible. While some progress has been made in combating the progression to, and complications of, ESRD, the clinical benefits of existing interventions remain limited.

Arterial hypertension is a major health problem worldwide. Treatment-resistant hypertension is defined as the failure to achieve target blood pressure despite the concomitant use of maximally tolerated doses of three different antihypertensive medications, including a diuretic. Treatment-resistant hypertension is associated with considerable morbidity and mortality. Patients with treatment-resistant hypertension have markedly increased cardiovascular morbidity and mortality, facing an increase in the risk of myocardial infarction (MI), stroke, and death compared to patients whose hypertension is adequately controlled.

The autonomic nervous system is recognized as an important pathway for control signals that are responsible for the regulation of body functions critical for maintaining vascular fluid balance and blood pressure. The autonomic nervous system conducts information in the form of signals from the body's biologic sensors such as baroreceptors (responding to pressure and volume of blood) and chemoreceptors (responding to chemical composition of blood) to the central nervous system via its sensory fibers. It also conducts command signals from the central nervous system that control the various innervated components of the vascular system via its motor fibers.

It is known from clinical experience and research that an increase in renal sympathetic nerve activity leads to vasoconstriction of blood vessels supplying the kidney, decreased renal blood flow, decreased removal of water and sodium from the body, and increased renin secretion. It is also known that reduction of sympathetic renal nerve activity, e.g., via denervation, may reverse these processes.

The renal sympathetic nervous system plays a critical influence in the pathophysiology of hypertension. The adventitia of the renal arteries has efferent and afferent sympathetic nerves. Renal sympathetic activation via the efferent nerves initiates a cascade resulting in elevated blood pressure. Efferent sympathetic outflow leads to vasoconstriction with a subsequent reduction in glomerular blood flow, a lowering of the glomerular filtration rate, release of renin by the juxtaglomerular cells, and the subsequent activation of the renin-angiotensin-aldosterone axis leading to increased tubular reabsorption of sodium and water. Decreased glomerular filtration rate also prompts additional systemic sympathetic release of catecholamines. As a consequence, blood pressure increases by a rise in total blood volume and increased peripheral vascular resistance.

The system 100 can be used for the treatment of cardio-renal diseases, including hypertension, by providing renal neuromodulation and/or denervation. For example, the device 102 may be placed at one or more target sites associated with renal nerves other neural fibers that contribute to renal neural function, or other neural features. For example, the device 102, in conjunction with the console 104, may detect, identify, and precision target renal nerve tissue and subsequently deliver energy at a level or frequency sufficient to therapeutically modulate nerves associated with such renal tissue. The therapeutic modulation of such renal nerves and/or renal tissue is sufficient to completely block or denervate the target neural structures and/or disrupt renal nervous activity, while minimizing and/or preventing collateral damage to surrounding or adjacent non-neural tissue including bloods vessels and bone and non-targeted neural tissue.

It should further be noted that the system 100 can be used to determine disease progression. In particular, the present system 100 can obtain measurements at one or more target sites associated with a given disease, disorder, or the like. Such measurements may be based on the active neural parameters (i.e., neuronal firing and active voltage monitoring) and may be used to identify neurons. The active neural parameters (and thus behavior) change with disease progression, thereby allowing the present system to identify such changes and determine a progression of the underlying disease or disorder. Such capabilities are possible based, at least in part, on the fact that the present system 100 is configured to monitor passive electric phenomena (i.e., the present system 100 determines the ohmic conductivity frequency, which remains consistent, while conductivity will be different based on disease or disorder progression).

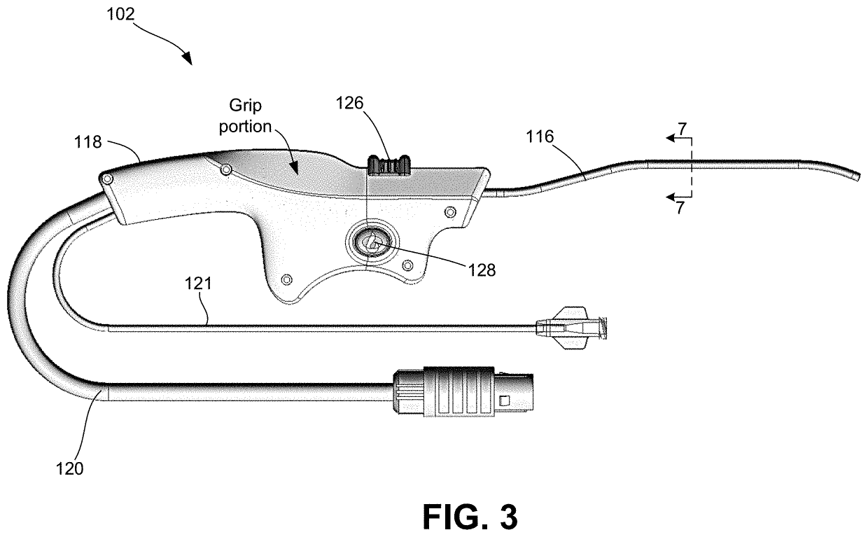

FIG. 3 is a side view of one embodiment of a handheld device for providing therapeutic neuromodulation consistent with the present disclosure. As previously described, the device 102 includes an end effector (not shown) transformable between a collapsed/retracted configuration and an expanded deployed configuration, a shaft 116 operably associated with the end effector, and a handle 118 operably associated with the shaft 116. The handle 118 includes at least a first mechanism 126 for deployment of the end effector from collapsed/retracted configuration to the expanded, deployed configuration, and a second mechanism 128, separate from the first mechanism 124, for control of energy output by the end effector, specifically electrodes or other energy elements provided by the end effector. The handheld device 102 may further include an auxiliary line 121, which may provide a fluid connection between a fluid source, for example, and the shaft 116 such that fluid may be provided to a target site via the distal end of the shaft 116. In some embodiments, the auxiliary line 121 may provide a connection between a vacuum source and the shaft 116, such that the device 102 may include suction capabilities (via the distal end of the shaft 116).

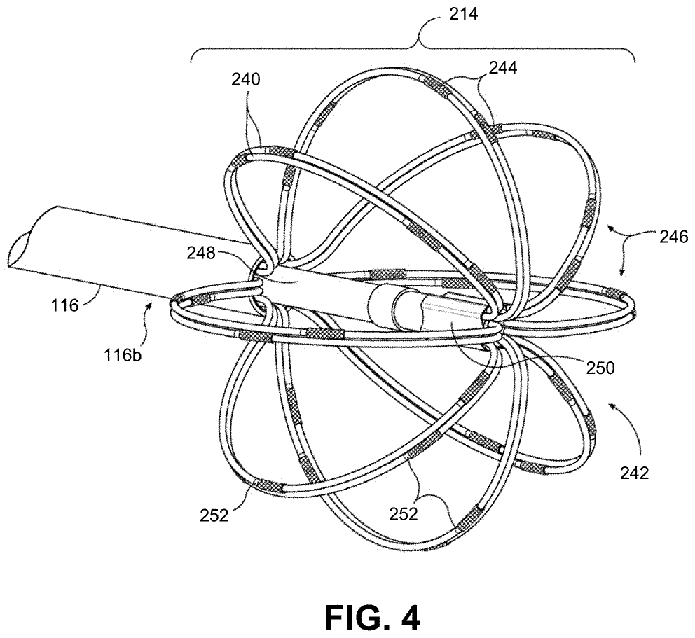

FIG. 4 is an enlarged, perspective view of one embodiment of an end effector 214 consistent with the present disclosure. As shown, the end effector 214 is generally positioned at a distal portion 116b of the shaft 116. The end effector 214 is transformable between a low-profile delivery state to facilitate intraluminal delivery of the end effector 214 to a treatment site and an expanded state, as shown. The end effector 214 includes a plurality of struts 240 that are spaced apart from each other to form a frame or basket 242 when the end effector 214 is in the expanded state. The struts 240 can carry one or more energy delivery elements, such as a plurality of electrodes 244. In the expanded state, the struts 240 can position at least two of the electrodes 244 against tissue at a target site within a particular region. The electrodes 244 can apply bipolar or multi-polar RF energy to the target site to therapeutically modulate nerves associated with a peripheral neurological condition or disorder. In various embodiments, the electrodes 244 can be configured to apply pulsed RF energy with a desired duty cycle (e.g., 1 second on/0.5 seconds off) to regulate the temperature increase in the target tissue.

In the embodiment illustrated in FIG. 4, the basket 242 includes eight branches 246 spaced radially apart from each other to form at least a generally spherical structure, and each of the branches 246 includes two struts 240 positioned adjacent to each other. In other embodiments, however, the basket 242 can include fewer than eight branches 246 (e.g., two, three, four, five, six, or seven branches) or more than eight branches 246. In further embodiments, each branch 246 of the basket 242 can include a single strut 240, more than two struts 240, and/or the number of struts 240 per branch can vary. In still further embodiments, the branches 246 and struts 240 can form baskets or frames having other suitable shapes for placing the electrodes 244 in contact with tissue at the target site. For example, when in the expanded state, the struts 240 can form an ovoid shape, a hemispherical shape, a cylindrical structure, a pyramid structure, and/or other suitable shapes.

The end effector 214 can further include an internal or interior support member 248 that extends distally from the distal portion 116b of the shaft 116. A distal end portion 250 of the support member 248 can support the distal end portions of the struts 240 to form the desired basket shape. For example, the struts 240 can extend distally from the distal potion 116b of the shaft 116 and the distal end portions of the struts 240 can attach to the distal end portion 250 of the support member 248. In certain embodiments, the support member 248 can include an internal channel (not shown) through which electrical connectors (e.g., wires) coupled to the electrodes 244 and/or other electrical features of the end effector 214 can run. In various embodiments, the internal support member 248 can also carry an electrode (not shown) at the distal end portion 250 and/or along the length of the support member 248.

The basket 242 can transform from the low-profile delivery state to the expanded state (shown in FIG. 4) by either manually manipulating a handle of the device 102, interacting with the first mechanism 126 for deployment of the end effector 214 from collapsed/retracted configuration to the expanded, deployed configuration, and/or other feature at the proximal portion of the shaft 116 and operably coupled to the basket 242. For example, to move the basket 242 from the expanded state to the delivery state, an operator can push the support member 248 distally to bring the struts 240 inward toward the support member 248. An introducer or guide sheath (not shown) can be positioned over the low-profile end effector 214 to facilitate intraluminal delivery or removal of the end effector 214 from or to the target site. In other embodiments, the end effector 214 is transformed between the delivery state and the expanded state using other suitable means, such as the first mechanism 126, as will be described in greater detail herein.

The individual struts 240 can be made from a resilient material, such as a shape-memory material (e.g., Nitinol) that allows the struts 240 to self-expand into the desired shape of the basket 242 when in the expanded state. In other embodiments, the struts 240 can be made from other suitable materials and/or the end effector 214 can be mechanically expanded via a balloon or by proximal movement of the support member 248. The basket 242 and the associated struts 240 can have sufficient rigidity to support the electrodes 244 and position or press the electrodes 244 against tissue at the target site. In addition, the expanded basket 242 can press against surrounding anatomical structures proximate to the target site and the individual struts 240 can at least partially conform to the shape of the adjacent anatomical structures to anchor the end effector 214 at the treatment site during energy delivery. In addition, the expansion and conformability of the struts 240 can facilitate placing the electrodes 244 in contact with the surrounding tissue at the target site.

At least one electrode 244 is disposed on individual struts 240. In the illustrated embodiment, two electrodes 244 are positioned along the length of each strut 240. In other embodiments, the number of electrodes 244 on individual struts 240 be only one, more than two, zero, and/or the number of electrodes 244 on the different struts 240 can vary. The electrodes 244 can be made from platinum, iridium, gold, silver, stainless steel, platinum-iridium, cobalt chromium, iridium oxide, polyethylenedioxythiophene (“PEDOT”), titanium, titanium nitride, carbon, carbon nanotubes, platinum grey, Drawn Filled Tubing (“DFT”) with a silver core made by Fort Wayne Metals of Fort Wayne, Ind., and/or other suitable materials for delivery RF energy to target tissue.

In certain embodiments, each electrode 444 can be operated independently of the other electrodes 244. For example, each electrode can be individually activated and the waveform, polarity and amplitude of each electrode can be selected by an operator or a control algorithm (e.g., executed by the controller 107 of FIG. 1A). Various embodiments of such independently controlled electrodes 244 are described in greater detail herein. The selective independent control of the electrodes 244 allows the end effector 214 to deliver RF energy to highly customized regions and to further create multiple micro-lesions to selectively modulate a target neural structure by effectively causing multi-point interruption of a neural signal due to the multiple micro-lesions. For example, a select portion of the electrodes 244 can be activated to target neural fibers in a specific region while the other electrodes 244 remain inactive. In certain embodiments, for example, electrodes 244 may be activated across the portion of the basket 242 that is adjacent to tissue at the target site, and the electrodes 244 that are not proximate to the target tissue can remain inactive to avoid applying energy to non-target tissue. Such configurations facilitate selective therapeutic modulation of nerves along a portion of a target site without applying energy to structures in other portions of the target site.

The electrodes 244 can be electrically coupled to an RF generator (e.g., the generator 106 of FIG. 1A) via wires (not shown) that extend from the electrodes 244, through the shaft 116, and to the RF generator. When each of the electrodes 244 is independently controlled, each electrode 244 couples to a corresponding wire that extends through the shaft 116. In other embodiments, multiple electrodes 244 can be controlled together and, therefore, multiple electrodes 244 can be electrically coupled to the same wire extending through the shaft 116. The RF generator and/or components operably coupled (e.g., a control module) thereto can include custom algorithms to control the activation of the electrodes 244. For example, the RF generator can deliver RF power at about 200-300 W to the electrodes 244, and do so while activating the electrodes 244 in a predetermined pattern selected based on the position of the end effector 214 relative to the treatment site and/or the identified locations of the target nerves. In other embodiments, the RF generator delivers power at lower levels (e.g., less than 1 W, 2-5 W, 5-15 W, 15-50 W, 50-150 W, etc.) and/or higher power levels.

The end effector 214 can further include one or more sensors 252 (e.g., temperature sensors, impedance sensors, etc.) disposed on the struts 240 and/or other portions of the end effector 214 and configured to sense/detect one or more properties associated with tissue at a target site. For example, temperature sensors are configured to detect the temperature adjacent thereto. The sensors 252 can be electrically coupled to a console (e.g., the console 104 of FIG. 1A) via wires (not shown) that extend through the shaft 116. In various embodiments, the sensors 252 can be positioned proximate to the electrodes 244 to detect various properties of targeted tissue and/or the treatment associated therewith. As will be described in greater detail herein, the sensed data can be provided to the console 104, wherein such data is generally related to at least bioelectric properties of tissue at the target site. In turn, the console 104 (via the controller 107, monitoring system 108, and evaluation/feedback algorithms 110) is configured to process such data and determine to identify a type of each of the one or more tissues at the target site and further identify a dielectric relaxation pattern for each of the one or more identified tissue types. The console (via the controller 107, monitoring system 108, and evaluation/feedback algorithms 110) is further configured to determine an ablation pattern to be delivered by one or more of the plurality of electrodes of the end effector based on identified dielectric relaxation patterns. The ablation energy associated with the ablation pattern is at a level sufficient to ablate a targeted tissue and minimize and/or prevent collateral damage to surrounding or adjacent non-targeted tissue at the target site.

In some embodiments, the device 102 may further be configured to provide the console 104 with sensed data in the form of feedback data associated with the effect of the therapeutic stimulation on the targeted tissue. For example, feedback data may be associated with efficacy of ablation upon targeted tissue (e.g., neural tissue) during and/or after delivery of initial energy from one or more of the plurality of electrodes. Accordingly, in certain embodiments, the console 104 (via the controller 107, monitoring system 108, and evaluation/feedback algorithms 110) is configured to process such feedback data to determine if certain properties of the targeted tissue undergoing treatment (e.g., tissue temperature, tissue impedance, etc.) reach predetermined thresholds for irreversible tissue damage. The controller 107 can tune energy output individually for the one or more electrodes after an initial level of energy has been delivered based, at least in part, on feedback data. For example, once the threshold is reached, the application of therapeutic neuromodulation energy can be terminated to allow the tissue to remain intact. In certain embodiments, the energy delivery can automatically be tuned based on an evaluation/feedback algorithm (e.g., the evaluation/feedback algorithm 110 of FIG. 1A) stored on a console (e.g., the console 104 of FIG. 1A) operably coupled to the end effector 214.

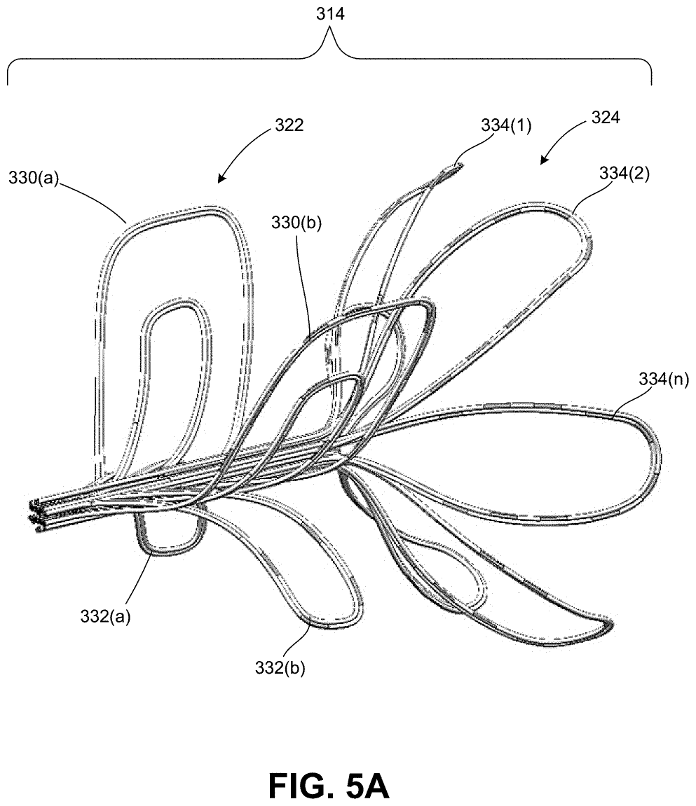

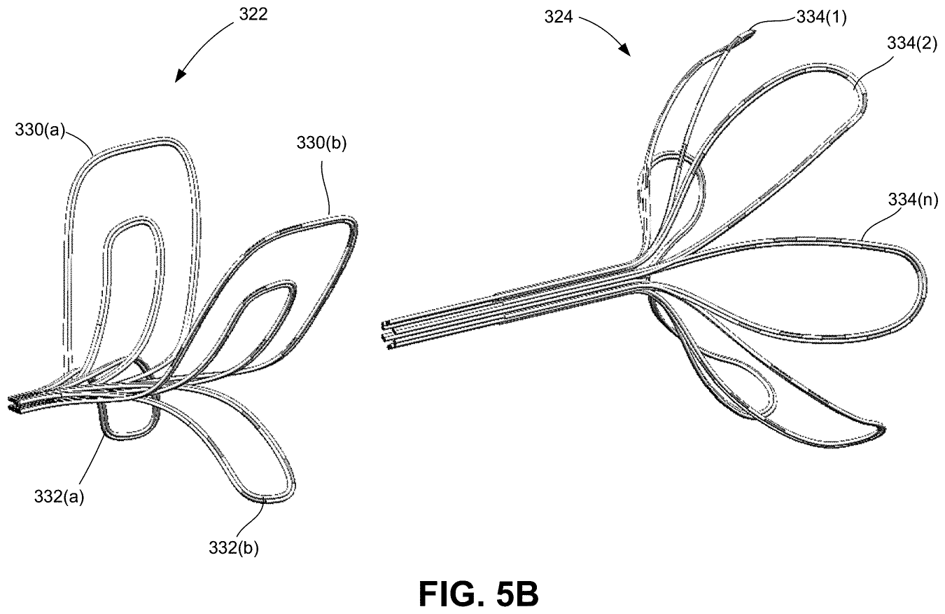

FIGS. 5A-5F are various views of another embodiment of an end effector 314 consistent with the present disclosure. As generally illustrated, the end effector 314 is a multi-segmented end effector, which includes at least a first segment 322 and a second segment 324 spaced apart from one another. The first segment 322 is generally positioned closer to a distal portion of the shaft 116, and is thus sometimes referred to herein as the proximal segment 322, while the second segment 324 is generally positioned further from the distal portion of the shaft 116 and is thus sometimes referred to herein as the distal segment 324. Each of the first and second segments 322 and 324 is transformable between a retracted configuration, which includes a low-profile delivery state and a deployed configuration, which includes an expanded state, as shown in the figures. The end effector 314 is generally designed to be positioned within a nasal region of the patient for the treatment of a rhinosinusitis condition while minimizing or avoiding collateral damage to surrounding tissue, such as blood vessels or bone. In particular, the end effector 314 is configured to be advanced within the nasal cavity and be positioned at one or more target sites generally associated with postganglionic parasympathetic fibers that innervate the nasal mucosa. In turn, the end effector 314 is configured to therapeutically modulate the postganglionic parasympathetic nerves.

It should be noted, however, that an end effector consistent with the present disclosure may be multi-segmented in a similar fashion as end effector 314 and may be used to provide treatment in other regions of the patient outside of the nasal cavity and thus is not limited to the particular design/configuration as the end effector 314 nor the intended treatment site (e.g., nasal cavity). Rather, other multi-segmented designs are contemplated for use in particular regions of a patient, particularly regions in which the use of multiple and distinct segments would be advantageous, as is the case with the end effector 314 design due to the anatomy of the nasal cavity.

FIG. 5A is an enlarged, perspective view of the multi-segment end effector illustrating the first (proximal) segment 322 and second (distal) segment 324. FIG. 5B is an exploded, perspective view of the multi-segment end effector 314. FIG. 5C is an enlarged, top view of the multi-segment end effector 314. FIG. 5D is an enlarged, side view of the multi-segment end effector 314. FIG. 5E is an enlarged, front (proximal facing) view of the first (proximal) segment 322 of the multi-segment end effector 314 and FIG. 5F is an enlarged, front (proximal facing) view of the second (distal) segment 324 of the multi-segment end effector 314.

As illustrated, the first segment 322 includes at least a first set of flexible support elements, generally in the form of wires, arranged in a first configuration, and the second segment 324 includes a second set of flexible support elements, also in the form of wires, arranged in a second configuration. The first and second sets of flexible support elements include composite wires having conductive and elastic properties. For example, in some embodiments, the composite wires include a shape memory material, such as Nitinol. The flexible support elements may further include a highly lubricious coating, which may allow for desirable electrical insulation properties as well as desirable low friction surface finish. Each of the first and second segments 322, 324 is transformable between a retracted configuration and an expanded deployed configuration such that the first and second sets of flexible support elements are configured to position one or more electrodes provided on the respective segments (see electrodes 336 in FIGS. 5E and 5F) into contact with one or more target sites when in the deployed configuration.

As shown, when in the expanded deployed configuration, the first set of support elements of the first segment 322 includes at least a first pair of struts 330a, 330b, each comprising a loop (or leaflet) shape and extending in an upward direction and a second pair of struts 332a, 332b, each comprising a loop (or leaflet) shape and extending in a downward direction, generally in an opposite direction relative to at least the first pair of struts 330a, 330b. It should be noted that the terms upward and downward are used to describe the orientation of the first and second segments 322, 324 relative to one another. More specifically, the first pair of struts 330a, 330b generally extend in an outward inclination in a first direction relative to a longitudinal axis of the multi-segment end effector 314 and are spaced apart from one another. Similarly, the second pair of struts 332a, 332b extend in an outward inclination in a second direction substantially opposite the first direction relative to the longitudinal axis of the multi-segment end effector and spaced apart from one another.

The second set of support elements of the second segment 324, when in the expanded deployed configuration, includes a second set of struts 334(1), 334(2), 334(n) (approximately six struts), each comprising a loop shape extending outward to form an open-ended circumferential shape. As shown, the open-ended circumferential shape generally resembles a blooming flower, wherein each looped strut 334 may generally resemble a flower petal. It should be noted that the second set of struts 334 may include any number of individual struts and is not limited to six, as illustrated. For example, in some embodiments, the second segment 124 may include two, three, four, five, six, seven, eight, nine, ten, or more struts 334.

The first and second segments 322, 324, specifically struts 330, 332, and 334 include one or more energy delivery elements, such as a plurality of electrodes 336. It should be noted that any individual strut may include any number of electrodes 336 and is not limited to one electrode, as shown. In the expanded state, the struts 330, 332, and 334 can position any number of electrodes 336 against tissue at a target site within the nasal region (e.g., proximate to the palatine bone inferior to the SPF). The electrodes 336 can apply bipolar or multi-polar radiofrequency (RF) energy to the target site to therapeutically modulate postganglionic parasympathetic nerves that innervate the nasal mucosa proximate to the target site. In various embodiments, the electrodes 336 can be configured to apply pulsed RF energy with a desired duty cycle (e.g., 1 second on/0.5 seconds off) to regulate the temperature increase in the target tissue.

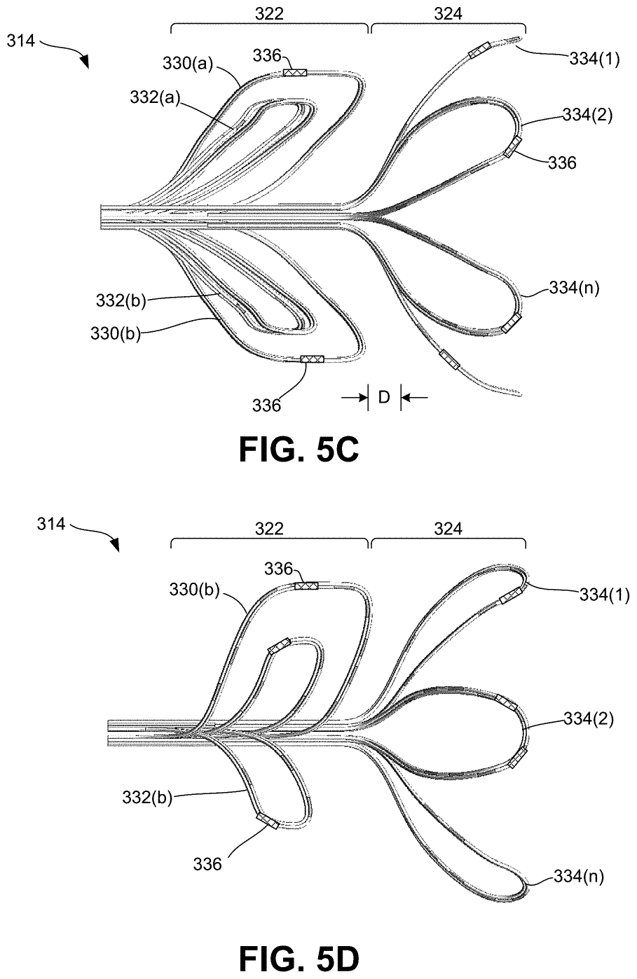

The first and second segments 322, 324 and the associated struts 330, 332, and 334 can have sufficient rigidity to support the electrodes 336 and position or press the electrodes 336 against tissue at the target site. In addition, each of the expanded first and second segments 322, 324 can press against surrounding anatomical structures proximate to the target site (e.g., the turbinates, the palatine bone, etc.) and the individual struts 330, 332, 334 can at least partially conform to the shape of the adjacent anatomical structures to anchor the end effector 314. In addition, the expansion and conformability of the struts 330, 332, 334 can facilitate placing the electrodes 336 in contact with the surrounding tissue at the target site. The electrodes 336 can be made from platinum, iridium, gold, silver, stainless steel, platinum-iridium, cobalt chromium, iridium oxide, polyethylenedioxythiophene (PEDOT), titanium, titanium nitride, carbon, carbon nanotubes, platinum grey, Drawn Filled Tubing (DFT) with a silver core, and/or other suitable materials for delivery RF energy to target tissue. In some embodiments, such as illustrated in FIG. 6, a strut may include an outer jacket surrounding a conductive wire, wherein portions of the outer jacket are selectively absent along a length of the strut, thereby exposing the underlying conductive wire so as to act as an energy delivering element (i.e., an electrode) and/or sensing element, as described in greater detail herein.

In certain embodiments, each electrode 336 can be operated independently of the other electrodes 336. For example, each electrode can be individually activated and the polarity and amplitude of each electrode can be selected by an operator or a control algorithm (e.g., executed by the controller 107 previously described herein). The selective independent control of the electrodes 336 allows the end effector 314 to deliver RF energy to highly customized regions. For example, a select portion of the electrodes 336 can be activated to target neural fibers in a specific region while the other electrodes 336 remain inactive. In certain embodiments, for example, electrodes 136 may be activated across the portion of the second segment 324 that is adjacent to tissue at the target site, and the electrodes 336 that are not proximate to the target tissue can remain inactive to avoid applying energy to non-target tissue. Such configurations facilitate selective therapeutic modulation of nerves on the lateral nasal wall within one nostril without applying energy to structures in other portions of the nasal cavity.

The electrodes 336 are electrically coupled to an RF generator (e.g., the generator 106 of FIG. 1A) via wires (not shown) that extend from the electrodes 336, through the shaft 116, and to the RF generator. When each of the electrodes 336 is independently controlled, each electrode 336 couples to a corresponding wire that extends through the shaft 116. In other embodiments, multiple electrodes 336 can be controlled together and, therefore, multiple electrodes 336 can be electrically coupled to the same wire extending through the shaft 116. As previously described, the RF generator and/or components operably coupled (e.g., a control module) thereto can include custom algorithms to control the activation of the electrodes 336. For example, the RF generator can deliver RF power at about 460-480 kHz (+ or −5 kHz) to the electrodes 336, and do so while activating the electrodes 336 in a predetermined pattern selected based on the position of the end effector 314 relative to the treatment site and/or the identified locations of the target tissues. It should further be noted that the electrodes 336 may be individually activated and controlled (i.e., controlled level of energy output and delivery) based, at least in part, on feedback data. The RF generator is able to provide bipolar low power (10 watts with maximum setting of 50 watts) RF energy delivery, and further provide multiplexing capabilities (across a maximum of 30 channels).

Once deployed, the first and second segments 322, 324 contact and conform to a shape of the respective locations, including conforming to and complementing shapes of one or more anatomical structures at the respective locations. In turn, the first and second segments 322, 324 become accurately positioned within the nasal cavity to subsequently deliver, via one or more electrodes 336, precise and focused application of RF thermal energy or non-thermal energy to the one or more target sites to thereby therapeutically modulate associated neural tissue. More specifically, the first and second segments 322, 324 have shapes and sizes when in the expanded configuration that are specifically designed to place portions of the first and second segments 322, 324, and thus one or more electrodes associated therewith 336, into contact with target sites within nasal cavity associated with postganglionic parasympathetic fibers that innervate the nasal mucosa.

For example, the first set of flexible support elements of the first segment 322 conforms to and complements a shape of a first anatomical structure at the first location when the first segment 322 is in the deployed configuration and the second set of flexible support elements of the second segment 124 conforms to and complements a shape of a second anatomical structure at the second location when the second segment is in the deployed configuration. The first and second anatomical structures may include, but are not limited to, inferior turbinate, middle turbinate, superior turbinate, inferior meatus, middle meatus, superior meatus, pterygopalatine region, pterygopalatine fossa, sphenopalatine foramen, accessory sphenopalatine foramen(ae), and sphenopalatine micro-foramen(ae).

In some embodiments, the first segment 322 of the multi-segment end effector 314 is configured in a deployed configuration to fit around at least a portion of a middle turbinate at an anterior position relative to the middle turbinate and the second segment 324 of the multi-segment end effector is configured in a deployed configuration to contact a plurality of tissue locations in a cavity at a posterior position relative to the middle turbinate.

For example, the first set of flexible support elements of the first segment (i.e., struts 330 and 332) conforms to and complements a shape of a lateral attachment and posterior-inferior edge of the middle turbinate when the first segment 322 is in the deployed configuration and the second set of flexible support elements (i.e., struts 334) of the second segment 324 contact a plurality of tissue locations in a cavity at a posterior position relative to the lateral attachment and posterior-inferior edge of middle turbinate when the second segment 324 is in the deployed configuration. Accordingly, when in the deployed configuration, the first and second segments 322, 324 are configured to position one or more associated electrodes 336 at one or more target sites relative to either of the middle turbinate and the plurality of tissue locations in the cavity behind the middle turbinate. In turn, electrodes 336 are configured to deliver RF energy at a level sufficient to therapeutically modulate postganglionic parasympathetic nerves innervating nasal mucosa at an innervation pathway within the nasal cavity of the patient.

As illustrated in FIG. 5E, the first segment 322 comprises a bilateral geometry. In particular, the first segment 322 includes two identical sides, including a first side formed of struts 330a, 332a and a second side formed of struts 330b, 332b. This bilateral geometry allows at least one of the two sides to conform to and accommodate an anatomical structure within the nasal cavity when the first segment 322 is in an expanded state. For example, when in the expanded state, the plurality of struts 330a, 332a contact multiple locations along multiple portions of the anatomical structure and electrodes provided by the struts are configured to emit energy at a level sufficient to create multiple micro-lesions in tissue of the anatomical structure that interrupt neural signals to mucus producing and/or mucosal engorgement elements. In particular, struts 330a, 332a conform to and complement a shape of a lateral attachment and posterior-inferior edge of the middle turbinate when the first segment 322 is in the deployed configuration, thereby allowing for both sides of the anatomical structure to receive energy from the electrodes. By having this independence between first and second side (i.e., right and left side) configurations, the first segment 322 is a true bilateral device. By providing a bilateral geometry, the multi-segment end effector 314 does not require a repeat use configuration to treat the other side of the anatomical structure, as both sides of the structure are accounted at the same time due to the bilateral geometry. The resultant micro-lesion pattern can be repeatable and is predictable in both macro element (depth, volume, shape parameter, surface area) and can be controlled to establish low to high effects of each, as well as micro elements (the thresholding of effects within the range of the macro envelope can be controlled), as well be described in greater detail herein. The systems of the present invention are further able to establish gradients within allowing for control over neural effects without having widespread effect to other cellular bodies, as will be described in greater detail herein.

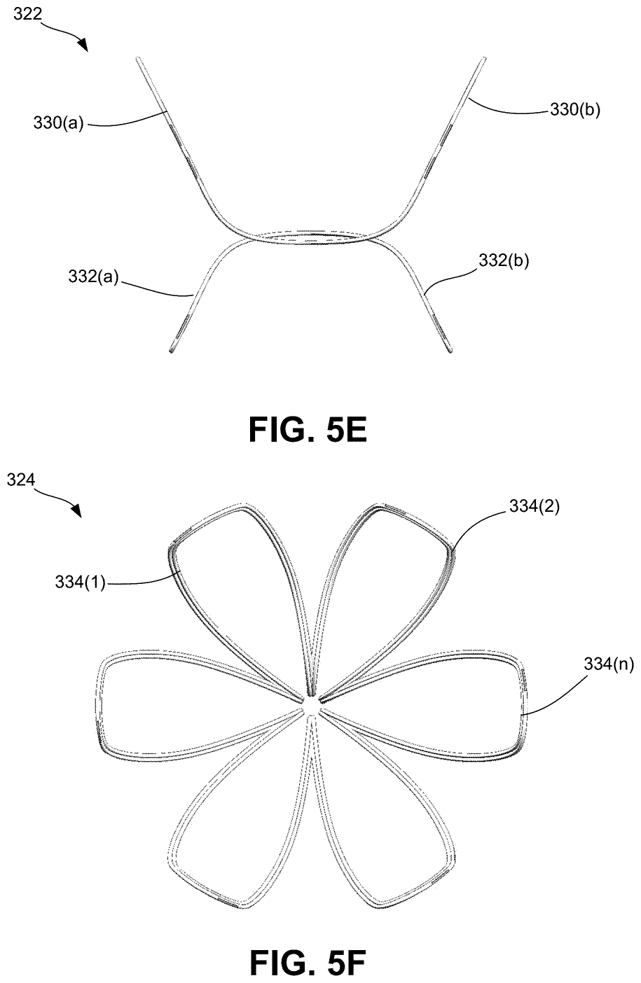

FIG. 7 is a cross-sectional view of a portion of the shaft 116 of the handheld device taken along lines 7-7 of FIG. 3. As illustrated, the shaft 116 may be constructed from multiple components so as to have the ability to constrain the end effector in the retracted configuration (i.e., the low-profile delivery state) when the end effector is retracted within the shaft 116, and to further provide an atraumatic, low profile and durable means to deliver the end effector to the target site. The shaft 116 includes coaxial tubes which travel from the handle 118 to a distal end of the shaft 116. The shaft 116 assembly is low profile to ensure adequate delivery of therapy in areas requiring low-profile access. The shaft 116 includes an outer sheath 138, surrounding a hypotube 140, which is further assembled over electrode wires 129 which surround an inner lumen 142. The outer sheath 138 serves as the interface between the anatomy and the device 102. The outer sheath 138 may generally include a low friction PTFE liner to minimize friction between the outer sheath 138 and the hypotube 140 during deployment and retraction. In particular the outer sheath 138 may generally include an encapsulated braid along a length of the shaft 116 to provide flexibility while retaining kink resistance and further retaining column and/or tensile strength. For example, the outer sheath 138 may include a soft Pebax material, which is atraumatic and enables smooth delivery through a passageway.

The hypotube 140 is assembled over the electrode wires starting within the handle 118 and traveling to the proximal end of the end effector. The hypotube 140 generally acts to protect the wires during delivery and is malleable to enable flexibility without kinking to thereby improve trackability. The hypotube 140 provides stiffness and enables torqueability of the device 102 to ensure accurate placement of the end effector 314. The hypotube 140 also provides a low friction exterior surface which enables low forces when the outer sheath 138 moves relative to the hypotube 140 during deployment and retraction or constraint. The shaft 116 may be pre-shaped in such a manner so as to complement a given anatomy (e.g., nasal cavity). For example, the hypotube 140 may be annealed to create a bent shaft 116 with a pre-set curve. The hypotube 140 may include a stainless-steel tubing, for example, which interfaces with a liner in the outer sheath 138 for low friction movement.

The inner lumen 142 may generally provide a channel for fluid extraction during a treatment procedure. For example, the inner lumen 142 extends from the distal end of the shaft 116 through the hypotube 140 and to atmosphere via a fluid line (line 121 of FIG. 3). The inner lumen 142 materials are chosen to resist forces of external components acting thereon during a procedure.

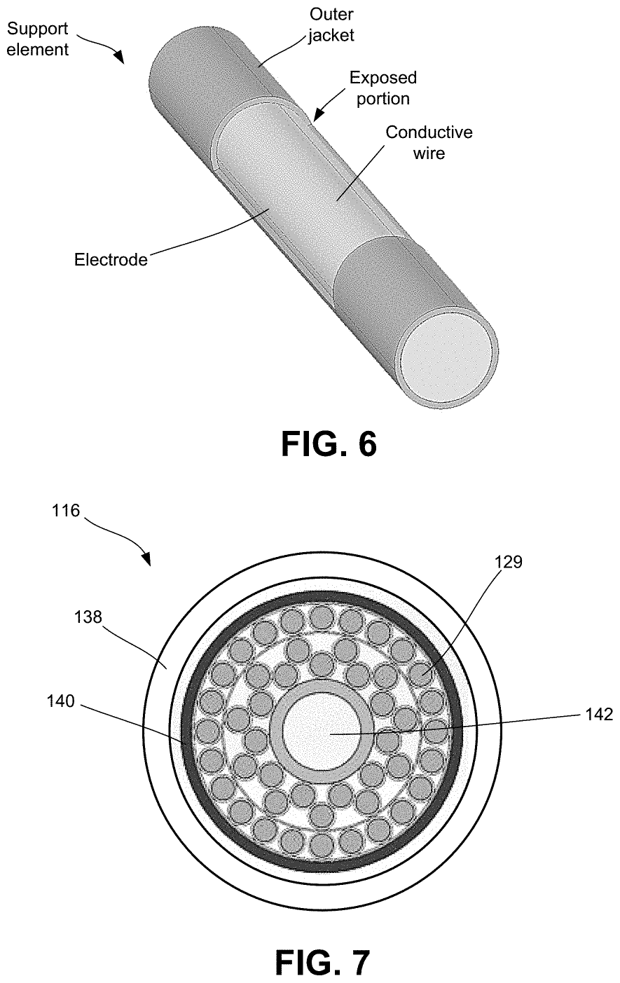

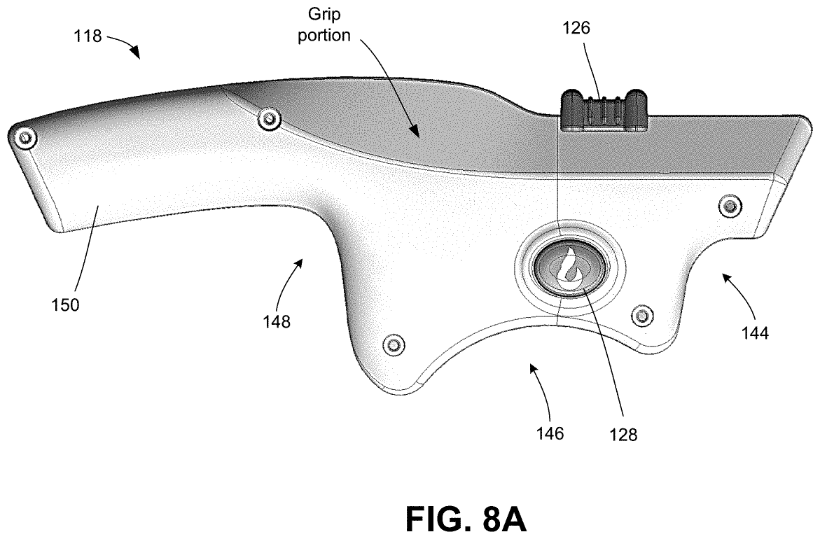

FIG. 8A is a side view of the handle of the handheld 118 and FIG. 8B is a side view of the handle 118 illustrating internal components enclosed within. The handle 118 generally includes an ergonomically-designed grip portion which provides ambidextrous use for both left and right handed use and conforms to hand anthropometrics to allow for at least one of an overhand grip style and an underhand grip style during use in a procedure. For example, the handle 118 may include specific contours, including recesses 144, 146, and 148 which are designed to naturally receive one or more of an operator's fingers in either of an overhand grip or underhand grip style and provide a comfortable feel for the operator. For example, in an underhand grip, recess 144 may naturally receive an operator's index finger, recess 146 may naturally receive an operator's middle finger, and recess 148 may naturally receive an operator's ring and little (pinkie or pinky) fingers which wrap around the proximal protrusion 150 and the operator's thumb naturally rests on a top portion of the handle 118 in a location adjacent to the first mechanism 126. In an overhand grip, the operator's index finger may naturally rest on the top portion of the handle 118, adjacent to the first mechanism 126, while recess 144 may naturally receive the operator's middle finger, recess 146 may naturally receive a portion of the operator's middle and/or ring fingers, and recess 148 may naturally receive and rest within the space (sometimes referred to as the purlicue) between the operator's thumb and index finger.

As previously described, the handle includes multiple user-operated mechanisms, including at least a first mechanism 126 for deployment of the end effector from the collapsed/retracted configuration to the expanded deployed configuration and a second mechanism 128 for controlling of energy output by the end effector, notably energy delivery from one or more electrodes. As shown, the user inputs for the first and second mechanisms 126, 128 are positioned a sufficient distance to one another to allow for simultaneous one-handed operation of both user inputs during a procedure. For example, user input for the first mechanism 126 is positioned on a top portion of the handle 118 adjacent the grip portion and user input for the second mechanism 128 is positioned on side portions of the handle 118 adjacent the grip portion. As such, in an underhand grip style, the operator's thumb rests on the top portion of the handle adjacent to the first mechanism 126 and at least their middle finger is positioned adjacent to the second mechanism 128, each of the first and second mechanisms 126, 128 accessible and able to be actuated. In an overhand grip system, the operator's index finger rests on the top portion of the handle adjacent to the first mechanism 126 and at least their thumb is positioned adjacent to the second mechanism 128, each of the first and second mechanisms 126, 128 accessible and able to be actuated. Accordingly, the handle accommodates various styles of grip and provides a degree of comfort for the surgeon, thereby further improving execution of the procedure and overall outcome.

Referring to FIG. 8B, the various components provided within the handle 118 are illustrated. As shown, the first mechanism 126 may generally include a rack and pinion assembly providing movement of end effector between the retracted and deployed configurations in response to input from a user-operated controller. The rack and pinion assembly generally includes a set of gears 152 for receiving input from the user-operated controller and converting the input to linear motion of a rack member 154 operably associated with at least one of the shaft 116 and the end effector. The rack and pinion assembly comprises a gearing ratio sufficient to balance a stroke length and retraction and deployment forces, thereby improving control over the deployment of the end effector. As shown, the rack member 154 may be coupled to a portion of the shaft 116, for example, such that movement of the rack member 154 in a direction towards a proximal end of the handle 118 results in corresponding movement of the shaft 116 while the end effector remains stationary, thereby exposing the end effector and allowing the end effector to transition from the constrained, retracted configuration to the expanded, deployed configuration. Similarly, movement of the rack member 154 in a direction towards a distal end of the handle 118 results in corresponding movement of the shaft 116 while the end effector remains stationary, and thereby encloses the end effector within the shaft 116. It should be noted that, in other embodiments, the rack member 154 may be directly coupled to a portion of the end effector such that movement of the rack member 154 results in corresponding movement of the end effector while the shaft 116 remains stationary, thereby transitioning the end effector between the retracted and deployed configurations.

The user-operated controller associated with the first mechanism 126 may include a slider mechanism operably associated with the rack and pinion rail assembly. Movement of the slider mechanism in a rearward direction towards a proximal end of the handle results in transitioning of the end effector to the deployed configuration and movement of the slider mechanism in a forward direction towards a distal end of the handle results in transitioning of the end effector to the retracted configuration. In other embodiment, the user-operated controller associated with the first mechanism 126 may include a scroll wheel mechanism operably associated with the rack and pinion rail assembly. Rotation of the wheel in a rearward direction towards a proximal end of the handle results in transitioning of the end effector to the deployed configuration and rotation of the wheel in a forward direction towards a distal end of the handle results in transitioning of the end effector to the retracted configuration.

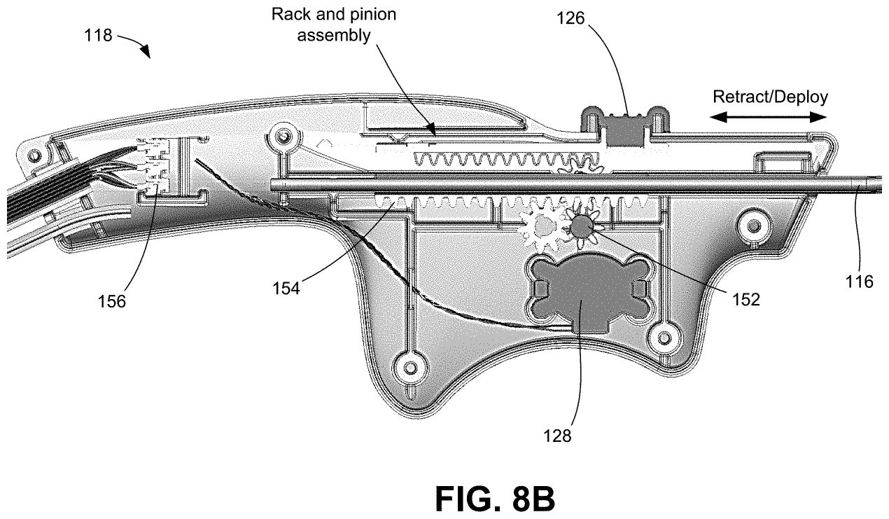

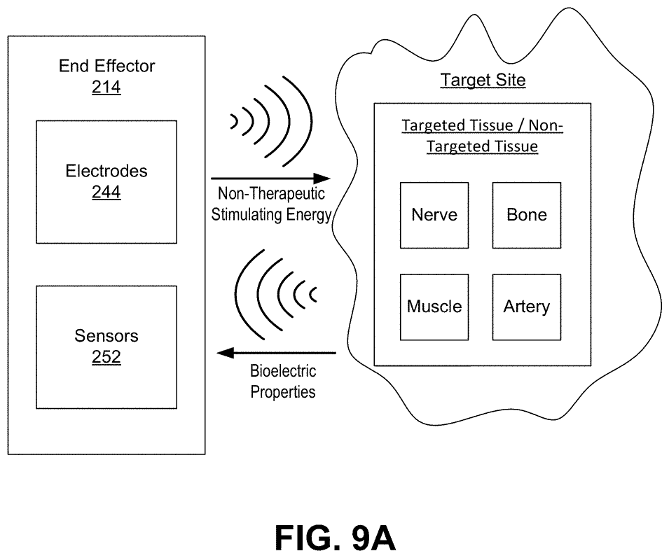

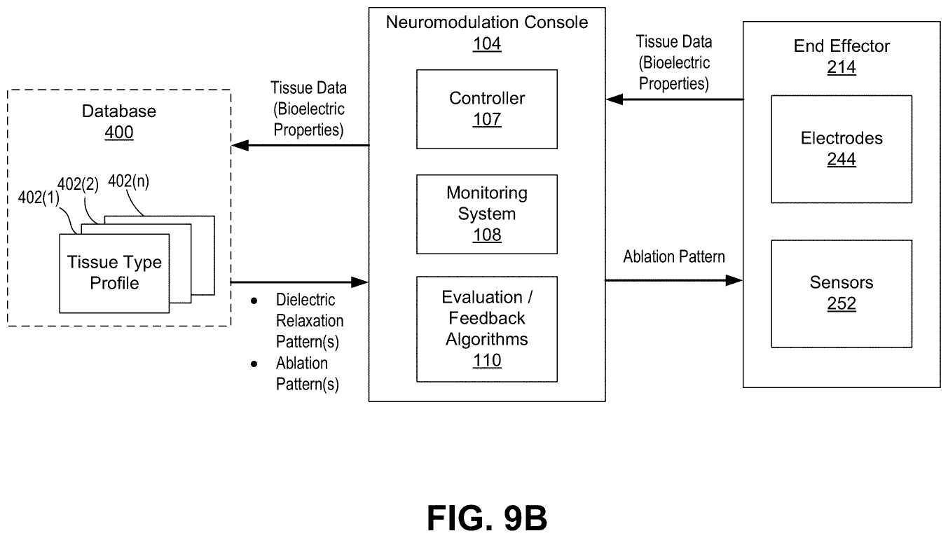

FIGS. 9A, 9B, and 9C are block diagrams illustrating the process of sensing, via an end effector, data associated with one or more tissues at a target site, notably bioelectric electric properties of one more tissues at the target site, and the subsequent processing of such data (via the controller 107, monitoring system 108, and evaluation/feedback algorithms 110) to determine the type of tissue(s) at the target site and further identify a dielectric relaxation pattern for each of the one or more identified tissue types, and further determining an ablation pattern to be delivered by one or more of the plurality of electrodes of the end effector based on identified dielectric relaxation patterns. The ablation energy associated with the ablation pattern is at a level sufficient to ablate a targeted tissue and minimize and/or prevent collateral damage to surrounding or adjacent non-targeted tissue at the target site.

It should be noted that, while the block diagrams of FIGS. 9A, 9B, and 9C include reference to end effector 214, other end effector embodiments, including end effector 314, are similarly configured with respect to sensing data associated with at least the presence of neural tissue and other properties of the neural tissue, including neural tissue depth. Accordingly, the following process is not limited to end effector 214.

FIG. 9A is a block diagram illustrating delivery of non-therapeutic energy from electrodes 244 of the end effector at a frequency for sensing one or more properties associated with tissue at a target site in response to the non-therapeutic energy.

As previously described, the handheld treatment device includes an end effector comprising a micro-electrode array arranged about a plurality of struts. For example, end effector 214 includes a plurality of struts 240 that are spaced apart from each other to form a frame or basket 242 when the end effector 214 is in the expanded state. The struts 240 include a plurality of energy delivery elements, such as a plurality of electrodes 244. In the expanded state, each of the plurality of struts is able to conform to and accommodate an anatomical structure at a target site. When positioned, the struts may contact multiple locations along multiple portions of a target site and thereby position one or more electrodes 244 against tissue at a target site. At least a subset of electrodes is configured to deliver non-therapeutic stimulating energy at a frequency/waveform to respective positions at the target site to thereby sense the bioelectric properties of the one or more tissues at the target site, and further convey such data to the console 104. In addition to bioelectric properties, the data may also include at least one of physiological properties and thermal properties of tissue at the target site.

For example, upon delivering non-therapeutic stimulating energy (via one or more electrodes 244) to respective positions, various properties of the tissue at the one or more target sites can be detected. This information can then be transmitted to the console 104, particularly the controller 107, monitoring system 108, and evaluation/feedback algorithms 110 to determine the anatomy at the target site (e.g., tissue types, tissue locations, vasculature, bone structures, foramen, sinuses, etc.), locate a tissue of interest (targeted tissue to receive electric therapeutic stimulation), such as neural tissue, differentiate between different types of neural tissue, and map the anatomical and/or neural structure at the target site. For example, the end effector 214 can be used to detect resistance, complex electrical impedance, dielectric properties, temperature, and/or other properties that indicate the presence of neural fibers and/or other anatomical structures in the target region. In certain embodiments, the end effector 214, together with the console 104 components, can be used to determine resistance (rather than impedance) of the tissue (i.e., the load) to more accurately identify the characteristics of the tissue. For example, the evaluation/feedback algorithms 110 can determine resistance of the tissue by detecting the actual power and current of the load (e.g., via the electrodes 244).

In some embodiments, the system 100 provides resistance measurements with a high degree of accuracy and a very high degree of precision, such as precision measurements to the hundredths of an Ohm (e.g., 0.01Ω) for the range of 1-50Ω. The high degree of resistance detection accuracy provided by the system 100 allows for the detection sub-microscale structures, including the firing of neural tissue, differences between neural tissue and other anatomical structures (e.g., blood vessels), and even different types of neural tissue. This information can be analyzed by the evaluation/feedback algorithms 110 and/or the controller 107 and communicated to the operator via a high resolution spatial grid (e.g., on the display 112) and/or other type of display to identify neural tissue and other anatomy at the treatment site and/or indicate predicted neuromodulation regions based on the ablation pattern with respect to the mapped anatomy.

As previously described, in certain embodiments, each electrode 244 can be operated independently of the other electrodes 244. For example, each electrode can be individually activated and the polarity and amplitude of each electrode can be selected by an operator or a control algorithm executed by the controller 107. The selective independent control of the electrodes 244 allows the end effector 214 to detect information (i.e., the presence of neural tissue, depth of neural tissue, and other physiological and bioelectrical properties) and subsequently deliver RF energy to highly customized regions. For example, a select portion of the electrodes 244 can be activated to target specific neural fibers in a specific region while the other electrodes 244 remain inactive. In addition, the electrodes 244 can be individually activated to stimulate or therapeutically modulate certain regions in a specific pattern at different times (e.g., via multiplexing), which facilitates detection of anatomical parameters across a zone of interest and/or regulated therapeutic neuromodulation.

As previously described, the system 100 can identify tissue type of one or more tissues at a target site and further identify a dielectric relaxation pattern for each of the one or more identified tissue types prior to therapy such that the therapeutic stimulation can be applied to precise regions including targeted tissue, while avoiding negative effects on non-targeted tissue and structures (e.g., blood vessels). For example, the system 100 can detect various bioelectrical parameters in an interest zone to determine the location and morphology of various tissue types (e.g., different types of neural tissue, neuronal directionality, etc.) and/or other tissue (e.g., glandular structures, vessels, bony regions, etc.). The system 100 is further configured to measure bioelectric potential.

To do so, one or more of the electrodes 244 is placed in contact with an epithelial surface at a region of interest (e.g., a treatment site). Electrical stimuli (e.g., constant or pulsed currents at one or more frequencies, and/or alternating (sine, square, triangle, sawtooth, etc.) wave or direct constant current/power/voltage source at one or more frequencies) are applied to the tissue by one or more electrodes 244 at or near the treatment site, and the voltage and/or current differences based on the wave applied at various different frequencies between various pairs of electrodes 244 of the end effector 214 may be measured to produce a spectral profile or map of the detected bioelectric potential, which can be used to identify different types of tissues (e.g., vessels, neural tissue, and/or other types of tissue) in the region of interest. For example, a fixed current (i.e., direct or alternating current) can be applied to a pair of electrodes 244 adjacent to each other and the resultant voltages and/or currents between other pairs of adjacent electrodes 244 are measured. Conversely, a fixed voltage (i.e. mono or bi-phasic) can be applied to a pair of electrodes 244 adjacent to each other and the resultant current between other pairs of adjacent electrodes 244 are measured. It will be appreciated that the current injection electrodes 244 and measurement electrodes 244 need not be adjacent, and that modifying the spacing between the two current injection electrodes 244 can affect the depth of the recorded signals. For example, closely-spaced current injection electrodes 244 provided recorded signals associated with tissue deeper from the surface of the tissue than further spaced apart current injection electrodes 244 that provide recorded signals associated with tissue at shallower depths. Recordings from electrode pairs with different spacings may be merged to provide additional information on depth and localization of anatomical structures.

Further, complex impedance and/or resistance measurements of the tissue at the region of interest can be detected directly from current-voltage data provided by the bioelectric potential measurements while differing levels of frequency currents are applied to the tissue (e.g., via the end effector 114), and this information can be used to map the neural and anatomical structures by the use of frequency differentiation reconstruction. In particular, current-voltage data may be observed with the difference in dielectric and conductive properties of tissue type when different levels of current frequencies are applied.

Furthermore, applying the stimuli at different frequencies will target different stratified layers or cellular bodies or clusters, which can further be used to identify specific tissue type and respective dielectric relaxation phenomena/behavior of the identified tissue types.

For example, different tissue types include different physiological and histological characteristics (e.g., cell components, extracellular proteins, etc.). As a result of the different characteristics, different tissue types have different associated bioelectrical properties and thus exhibit different associated behavior in response to application of energy applied thereto. It should be noted that active bioelectrical properties may generally include the influx and outflux of ions into and out of a cell, while passive bioelectrical properties may include resistive, capacitive, and inductive properties of the cell. One such behavior is referred to as dielectric relaxation phenomena. The energy conduction behavior of tissue differs with frequency/energy applied as tissue passive electrical components activate and de-active depending on frequency applied. This switching action of activating and deactivating of these electrically passive components depends on the energy and frequency applied is known as relaxation phenomena. This relaxation can take place either on ionic or dielectric or atomic or electronic level (highly dependent on frequency). For example, the ionic resistive component of a tissue is relative to active more than capacitive or inductive component of the tissue and, in dielectric, the capacitive component is relatively more active than resistive component.

As a result, the relaxation phenomena of a given tissue occurs at a particular electrical frequency in which the membranes of cells of the given tissue become permeable to thereby allow electrical stimulation current (at the particular frequency) to flow through the membrane to thereby elicit a desired effect upon the tissue. When a tissue is not exhibiting the dielectric relaxation phenomena (i.e., when electrical stimulation current is tuned to a different frequency that does not correlate to the dielectric relaxation phenomena), the membranes of cells of the given tissue are not permeable to that specific electrical stimulation current and thus do not elicit an effect.

For example, at relatively high signal frequencies (e.g., electrical injection or stimulation), for example, cell membranes of neural tissue do not impede current flow, and the current passes directly through the cell membranes. In this case, the resultant measurement (e.g., impedance, resistance, capacitance, and/or induction) is a function of the intracellular and extracellular tissue and liquids. At low signal frequencies, the membranes impede current flow to provide different defining characteristics of the tissues, such as the shapes and morphologies of the cells, cell density and/or cell spacing. The stimulation frequencies can be in the megahertz range, in the kilohertz range (e.g., 400-500 kHz, 450-480 kHz, etc.), in the hertz range (e.g., 0.2-0.8 Hz, 8-12 Hz, etc.), and/or other frequencies attuned to the tissue being stimulated and the characteristics of the device being used. The detected complex impedance or resistances levels from the zone of interest can be displayed to the user (e.g., via the display 112) to visualize certain structures based on the stimulus frequency.

Further, the inherent morphology and composition of the anatomical structures within a given region or zone of a patient's body react differently to different frequencies and, therefore, specific frequencies can be selected to identify very specific structures. For example, the morphology or composition of targeted structures for anatomical mapping may depend on whether the cells of tissue or other structure are membranonic, stratified, and/or annular. In various embodiments, the applied stimulation signals can have predetermined frequencies attuned to specific neural tissue, such as the level of myelination and/or morphology of the myelination. For example, second axonal parasympathetic structures are poorly myelinated than sympathetic nerves or other structures and, therefore, will have a distinguishable response (e.g., complex impedance, resistance, etc.) with respect to a selected frequency than sympathetic nerves. Accordingly, applying signals with different frequencies to the target site can distinguish the targeted parasympathetic nerves from the non-targeted sensory nerves, and therefore provide highly specific target sites for neural mapping before or after therapy and/or neural evaluation post-therapy.

In some embodiments, the neural and/or anatomical mapping includes measuring data at a region of interest with at least two different frequencies to identify certain anatomical structures such that the measurements are taken first based on a response to an injection signal having a first frequency and then again based on an injection signal having a second frequency different from the first. For example, there are two frequencies at which hypertrophied (i.e., disease-state characteristics) sub-mucosal targets have a different electrical conductivity or permittivity compared to “normal” (i.e., healthy) tissue. Complex conductivity may be determined based on one or more measured physiological parameters (e.g., complex impedance, resistance, dielectric measurements, dipole measurements, etc.) and/or observance of one or more confidently known attributes or signatures. Furthermore, the system 100 can also apply neuromodulation energy via the electrodes 244 at one or more predetermined frequencies attuned to a target neural structure to provide highly targeted ablation of the selected neural structure associated with the frequency(ies). This highly targeted neuromodulation also reduces the collateral effects of neuromodulation therapy to non-target sites/structures (e.g., blood vessels) because the targeted signal (having a frequency tuned to a target neural structure) will not have the same modulating effects on the non-target structures.

Accordingly, passive bioelectric properties, such as complex impedance and resistance, can be used by the system 100 before, during, and/or after neuromodulation therapy to guide one or more treatment parameters. For example, before, during, and/or after treatment, impedance or resistance measurements may be used to confirm and/or detect contact between one or more electrodes 244 and the adjacent tissue. The impedance or resistance measurements can also be used to detect whether the electrodes 244 are placed appropriately with respect to the targeted tissue type by determining whether the recorded spectra have a shape consistent with the expected tissue types and/or whether serially collected spectra were reproducible. In some embodiments, impedance or resistance measurements may be used to identify a boundary for the treatment zone (e.g., specific neural tissue that are to be disrupted), anatomical landmarks, anatomical structures to avoid (e.g., vascular structures or neural tissue that should not be disrupted), and other aspects of delivering energy to tissue.

The bioelectric information can be used to produce a spectral profile or map of the different anatomical features tissues at the target site, and the anatomical mapping can be visualized in a 3D or 2D image via the display 112 and/or other user interface to guide the selection of a suitable treatment site. This neural and anatomical mapping allows the system 100 to accurately detect and therapeutically modulate neural fibers associated with certain neurological conditions or disorders to be treated. In addition, anatomical mapping also allows the clinician to identify certain structures that the clinician may wish to avoid during therapeutic neural modulation (e.g., certain arteries). The neural and anatomical bioelectric properties detected by the system 100 can also be used during and after treatment to determine the real-time effect of the therapeutic neuromodulation on the treatment site. For example, the evaluation/feedback algorithms 110 can also compare the detected neural locations and/or activity before and after therapeutic neuromodulation, and compare the change in neural activity to a predetermined threshold to assess whether the application of therapeutic neuromodulation was effective across the treatment site.

FIG. 9B is a block diagram illustrating communication of sensor data from the handheld device 102 to the controller and subsequent tuning, via the controller, of energy output based on the sensor data for precision targeting of tissue of interested and to be treated. As shown, the end effector 214 communicates the tissue data (i.e., bioelectric properties of tissue at the target site) to the console 104. The bioelectric properties may include, but are not limited to, complex impedance, resistance, reactance, capacitance, inductance, permittivity, conductivity, dielectric properties, muscle or nerve firing voltage, muscle or nerve firing current, depolarization, hyperpolarization, magnetic field, induced electromotive force, and combinations thereof. The dielectric properties may include, for example, at least a complex relative dielectric permittivity.

In turn, console 104 (via the controller 107, monitoring system 108, and evaluation/feedback algorithms 110) is configured to process such data and determine a type of tissue at the target site, as well as other properties, including a dielectric relaxation pattern for each of the one or more identified tissue types. The console 104 (via the controller 107, monitoring system 108, and evaluation/feedback algorithms 110) is further configured to determine an ablation pattern to be delivered by one or more of the plurality of electrodes of the end effector based on identified dielectric relaxation patterns. The ablation energy associated with the ablation pattern is at a level sufficient to ablate a targeted tissue and minimize and/or prevent collateral damage to surrounding or adjacent non-targeted tissue at the target site. More specifically, the console 104 (via the controller 107, monitoring system 108, and evaluation/feedback algorithms 110) is configured to tune energy output (i.e., delivery of electrical therapeutic stimulation) based on the dielectric relaxation pattern of a tissue of interest such that the energy delivered is at a specific frequency configured to target the tissue of interest while avoiding the non-targeted tissue (i.e., the energy is tuned to a frequency level associated with the dielectric relaxation phenomena of the targeted tissue only thereby penetrate the cell membranes of the targeted tissue).

The console 104 (via the controller 107, monitoring system 108, and evaluation/feedback algorithms 110) is generally configured to determine/calculate a dielectric relaxation pattern of a given identified tissue type based on an algorithm utilizing complex relative dielectric permittivity calculations in empirical modeling of relaxation phenomena.

For example, by way of background, a dielectric material is an electrical insulator that can be polarized by an applied electric field. When a dielectric material is placed in an electric field, electric charges do not flow through the material as they do in an electrical conductor, but only slightly shift from their average equilibrium positions causing dielectric polarization. Because of dielectric polarization, positive charges are displaced in the direction of the field and negative charges shift in the direction opposite to the field (for example, if the field is moving in the positive x-axis, the negative charges will shift in the negative x-axis). As a result, dielectric polarization creates an internal electric field that reduces the overall field within the dielectric itself. If a dielectric is composed of weakly bonded molecules, those molecules not only become polarized, but also reorient so that their symmetry axes align to the field.

Accordingly, biological tissue, most notably cells of biological tissue, can essentially be modeled as capacitors with dielectric properties. For example, the phospholipid bilayer of a cell membrane can resemble a parallel-plate capacitor, such that, depending on the frequency applied, a cell membrane will allow an electrical charge/current to flow through. Adding a dielectric allows the capacitor to store more charge for a given potential difference. For example, when a dielectric is inserted into a charged capacitor to increase the capacitance of the capacitor, the dielectric is polarized by the field. The electric field from the dielectric will partially cancel the electric field from the charge on the capacitor plates.

The resulting concepts of relative dielectric permittivity and dielectric constant can be used to further determine complex relative dielectric permittivity of a tissue for the subsequent calculation of dielectric relaxation phenomena of a given tissue. The dielectric constant or relative dielectric permittivity, is understood by the following formula:

$$κ = {ɛ}_{r} = \frac{{ɛ}_{m}}{{ɛ}_{0}}$$

Dielectric permittivity (ε) is the ability of a substance to hold an electrical charge and is a function of frequency, temperature, humidity and other physical parameters. The dielectric constant (κ), also referred to as relative dielectric permittivity (εᵣ), is the ratio of the permittivity of a substance to free space. In the above formula, εₘ is the complex frequency-dependent permittivity of the material and εₒ is the vacuum permittivity. The value of εₒ is 8.85418782×10⁻¹² m⁻³ kg⁻¹ s⁴ A². Many materials have an ε or κ. For example, the κ or εᵣ of air is 1, of water is approximately 80, of glass is between 5 and 10, of paper is between 2 and 4, and of body tissue is approximately 8 at a frequency of 1 kHz and at room temperature of 20 degrees Celsius (° C.).

By knowing the relative dielectric permittivity of a material, the complex relative dielectric permittivity may be obtained. The complex relative dielectric permittivity is understood by the following formula:

εᵣ=ε′ᵣ−jε′ᵣ

where εᵣ is the relative dielectric permittivity, or dielectric constant, ε′ᵣ is the real part of the complex dielectric constant, ε″ᵣ is the imaginary part of the complex dielectric constant, and j is the imaginary constant. The real part (ε′ᵣ) of the relative dielectric permittivity or dielectric constant defines the polarization capability of the material. The imaginary part (ε″ᵣ) of the relative dielectric permittivity or dielectric constant defines the loss of the material (ionic loss at low frequency around mHz to Hz range, dielectric heat loss at kHz to MHz range, atomic loss and electronic loss at higher frequencies) and the conducting behavior of the polymer. At low to middle frequency range, relaxation phenomena or behavior occurs when the dielectric material starts leaking charge or heat loss at a particular frequency, where the imaginary part of the dielectric constant becomes more dominant compared to that of the real part of the dielectric constant.

Certain parameters can be extracted from complex relative dielectric permittivity of a given tissue, including, for example, loss tangent (also referred to as dielectric loss) with respect to impedance measurements of a tissue. Dielectric loss quantifies a dielectric material's inherent dissipation of electromagnetic energy (e.g. heat). It can be parameterized in terms of either the loss angle δ or the corresponding loss tangent tan δ (i.e., loss tangent). Both refer to the phasor in the complex plane whose real and imaginary parts are the resistive (lossy) component of an electromagnetic field and its reactive (lossless) counterpart. Loss tangent is defined as:

$$tan δ = \frac{{ɛ}_{r}^{″}}{{ɛ}_{r}^{′}}$$

where δ always refers to the angle of complex dielectric permittivity and θ always refers to impedance phase angle, so tan θ=X_{c}/R. X_{c} is the reactive part of complex impedance and R is the real part of impedance. The relationship between loss tangent tan δ and impedance phase angle θ is: δ=90°−θ. Accordingly:

$$tan δ = cot θ = \frac{1}{2 π {R}_{p} {C}_{p}}$$

As previously described, the console 104 (via the controller 107, monitoring system 108, and evaluation/feedback algorithms 110) is generally configured to determine/calculate a dielectric relaxation pattern of a given identified tissue type based on an algorithm utilizing complex relative dielectric permittivity calculations in empirical modeling of relaxation phenomena. In some embodiments, the calculation of the dielectric relaxation pattern of a given identified tissue is based, at least in part, on the Havriliak-Negami relaxation model. The Havriliak-Negami relaxation is an empirical modification of the Debye relaxation model in electromagnetism. Unlike the Debye model, the Havriliak-Negami (HN) relaxation accounts for the asymmetry and broadness of the dielectric dispersion curve. The model was first used to describe the dielectric relaxation of some polymers, by adding two exponential parameters to the Debye equation:

$${E}^{*} = {E}_{∞} + ( {E}_{0} - {E}_{∞} ) \times \frac{1}{{[ 1 + {( i ω τ )}^{1 - {α}_{H N}} ]}^{{β}_{H N}}} - \frac{i {σ}_{d c}}{{ɛ}_{0} {ω}^{s}}$$

where ε_{∞} and ε₀ represent the total dielectric permittivity at high frequency and low frequency respectively. i is the characteristic complex number √−1, ω is the angular frequency (where ω=2πf), τ is the relaxation time and is given by 1/2πfₘₐₓ, fₘₐₓ is the peak frequency of loss modulus and α_{HN} and β_{HN} are shaped characteristics of the fitted curve which describe width and asymmetry of loss peak respectively where 0≤α_{HN}, β_{HN}≤1. The fitting parameters become 1 for purely ohmic conductivity and decrease with electrode polarization. The parameter ε₀-ε_{∞} denotes the dielectric strength (Δε) of nanocomposites. The exponents, α and β, describe the asymmetry and broadness of the corresponding spectra. where α_{HN}=0 the HN model reduces to the Cole-Davidson model. The HN relaxation model proposes that the real and imaginary parts of the complex relative permittivity can be expressed as a function of ω (angular frequency) and α and β, as follows:

$${ɛ}^{′} = {ɛ}_{∞} + ( {ɛ}_{0} - {ɛ}_{∞} ) \times \frac{cos ( {β}_{H N} θ )}{{[ 1 + 2 {( ω {τ}_{H N} )}^{1 - {α}_{HN}} sin ( \frac{{α}_{H N} π}{2} ) + {( ω τ )}^{2 ( 1 - α )} ]}^{\frac{β}{2}}}$$

$${ɛ}^{″} = ( {ɛ}_{0} - {ɛ}_{∞} ) \times \frac{sin ( {β}_{H N} θ )}{{[ 1 + 2 {( ω {τ}_{H N} )}^{1 - {α}_{HN}} sin ( \frac{{α}_{H N} π}{2} ) + {( ω τ )}^{2 ( 1 - α )} ]}^{\frac{β}{2}}}$$

$$and$$

$$θ = {tan}^{- 1} \frac{{( ωτ )}^{1 - {α}_{H N}} cos ( \frac{{ }^{{α}_{HN}} π}{2} )}{1 + {( ωτ )}^{1 - {α}_{H N}} sin ( \frac{{ }^{{α}_{HN}} π}{2} )}$$

Based on the above formulas, calculation of ε′ᵣ and ε″ᵣ as a function of ω (angular frequency) from the data using a capacitance equation, as well as dimensions of the electrodes, for example.

It should be noted that, in some embodiments, the system 100 may include a database 400 containing a plurality of profiles 402(1)-402(n) of identified and known tissue types, wherein each profile may include electric signature data for the associated tissue type. The electric signature data may generally include previously identified bioelectric properties of the tissue type and previously identified dielectric relaxation pattern, and the associated frequency at which the tissue type exhibits different dielectric/MWS/loss factor relaxation and or dielectric relaxation phenomena/behaviors. Accordingly, the console 104 (via the controller 107, monitoring system 108, and evaluation/feedback algorithms 110) is configured to process data received from the end effector 114 (i.e., bioelectric properties of one or more tissues at the target site) and determine a type of tissue at the target site a dielectric relaxation pattern for each of the one or more identified tissue types based on a comparison of the data with the electric signature data stored in each of the profiles 402. Upon a positive correlation between data sets, the console 104 is configured to identify a matching profile and thus determine the one or more tissue types at the target site and the respective relaxation and conductivity patterns of each so as to identify an accurate ablation pattern for limiting treatment to that of the targeted tissue.

As generally understood, in dielectric spectroscopy, large frequency dependent contributions to the dielectric response, especially at low frequencies, may come from build-ups of charge. This Maxwell-Wagner-Sillar polarization, occurs either at inner dielectric boundary layers on a mesoscopic scale, or at the external electrode-sample interface on a macroscopic scale. In both cases this leads to a separation of charges (such as through a depletion layer). The charges are often separated over a considerable distance (relative to the atomic and molecular sizes), and the contribution to dielectric loss can therefore be orders of magnitude larger than the dielectric response due to molecular fluctuations.

Maxwell-Wagner-Sillar polarization (also referred to as the Maxwell-Wagner effect) processes are taken into account during the investigation of inhomogeneous materials like suspensions or colloids, biological materials, phase separated polymers, blends, and crystalline or liquid crystalline polymers. The Maxwell-Wagner effect accounts for charge accumulation at the two-material interface on the basis of the difference of charge carrier relaxation times in these two materials. Macroscopically, basic electrical properties of materials are specified using two physical parameters, dielectric constant ϵ and conductivity ε. The ratio of these two parameters, τ=ε/σ. The simplest model for describing an inhomogenous structure is a double layer arrangement, where each layer is characterized by its permittivity ε′₁, ε′₂ and its conductivity σ₁, σ₂. The relaxation time for such an arrangement is given by:

$${τ}_{MW} = {ϵ}_{0} \frac{{ɛ}_{1} + {ɛ}_{2}}{{σ}_{1} + {σ}_{2}}$$

Importantly, since the materials' conductivities are in general frequency dependent, this shows that the double layer composite generally has a frequency dependent relaxation time even if the individual layers are characterized by frequency independent permittivities.

The system 100 of the present invention may utilize Maxwell-Wagner-Sillar (MWS) relaxation model to confirm the target frequencies (frequencies at which relaxation phenomena occurs) of identified tissues. As previously described, relaxation phenomena is important in understanding changes in electric behavior of tissues under different frequencies conditions. At molecular dynamics level, dielectric spectroscopy has proven to be a more improved technique compared to other measurement techniques including Nuclear Magnetic Resonance (NMR), Small angle X-ray scattering (SAXS), Dynamic Mechanical Analysis (DMA), Quasi-elastic light scattering and neutron scattering. Cooperative relaxation and Maxwell-Wagner-Sillar (MWS) polarization are the two types of relaxation phenomenon found in biological tissue at low frequency range. Cooperative relaxation occurs due to the relaxation of the backbone chain of biopolymers and is generally termed as glass transition relaxation of those biopolymers. Maxwell-Wagner-Sillar (MWS) relaxation occurs generally at a very low frequency in biological tissues due to charge trapping at the interface of materials having different permittivity-based molecules. Frequency-based MWS are hard to find using imaginary dielectric permittivity. However, electric modulus, an inverse of dielectric permittivity, εᵣ, can be used to define different relaxation especially MWS and crystalline loss in polymer and nanocomposites. Mathematically, it is presented by the following:

M*=1/εᵣ=1/(ε′−iε″)=ε′/(ε′²+ε″²)+jε″/(ε′²+ε″²)=M′+jM″

where M′ and M″ are the real and imaginary component of electric modulus, which is analogous to the shear modulus. ε′ and ε″ are real and img. dielectric permittivity of biological tissue.

FIG. 9C is a block diagram illustrating delivery of energy to the target site tuned to a specific frequency to elicit dielectric relaxation phenomena/behavior in the targeted tissue (based on the ablation pattern output from the controller). The level of energy output from the end effector may be at a therapeutic energy level sufficient to therapeutically modulate (e.g. ablate) the targeted tissue while minimizing and/or preventing damage to a surrounding or adjacent non-targeted tissue or structure. In particular, energy to be delivered from the end effector is tuned to a target frequency associated with a specific relaxation pattern of the targeted tissue. The target frequency is a frequency at which the targeted tissue exhibits near relaxation phenomena behavior and the non-targeted tissue does not exhibit relaxation phenomena behavior. In particular, delivery of the ablation energy, tuned to the target frequency, penetrates (passes through) a membrane of one or more cells associated only with the targeted tissue, while passing around cell membranes of non-targeted tissue and structures at the target site.

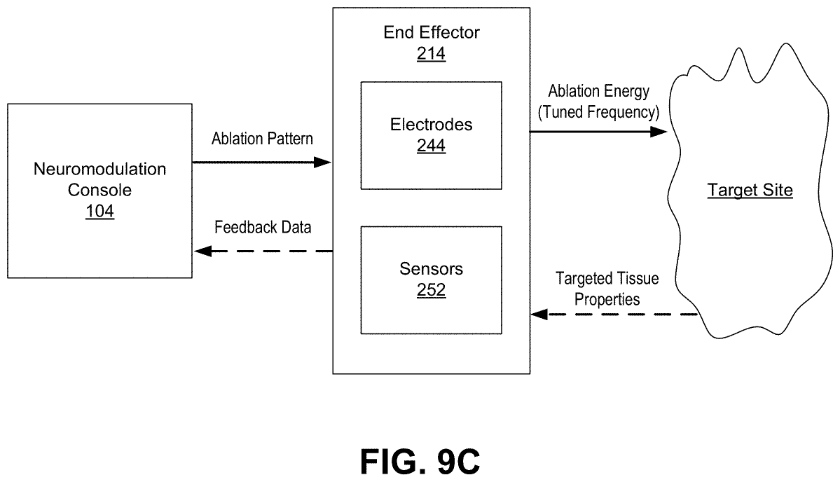

For example, in some embodiments, the condition to be treated may include a peripheral neurological condition. The peripheral neurological condition may be associated with a nasal condition, such as rhinosinusitis. Accordingly, in some embodiments, the target site is within a sino-nasal cavity (e.g., proximate or inferior to a sphenopalatine foramen) of the patient and the targeted tissue is neural tissue associated with rhinosinusitis (i.e., neural tissue innervating mucus producing and/or mucosal engorgement elements within the sino-nasal cavity). As a result, the ablation energy associated with the ablation pattern is at a level sufficient to ablate a targeted tissue (i.e., neural tissue) and minimize and/or prevent collateral damage to surrounding or adjacent non-targeted tissue at the target site. More specifically, as illustrated in FIG. 10, the energy output (i.e., delivery of electrical therapeutic stimulation) is based on the dielectric relaxation pattern of a tissue of interest (i.e., neural tissue in this instance) such that the energy delivered is at a specific frequency configured to target the tissue of interest while avoiding the non-targeted tissue (i.e., the energy is tuned to a frequency level associated with the dielectric relaxation phenomena of the targeted tissue only thereby penetrate the cell membranes of the targeted tissue).

FIG. 10 is a block diagram illustrating delivery of energy to the target site, and specifically illustrating flow of current through membranes of cells of the targeted tissue (near relaxation phenomena/behavior) and flow of current around membranes of cells of non-targeted tissue (which is not exhibiting nearby relaxation phenomena/behavior) as a result of the energy being tuned to a target frequency.

The delivery of the ablation energy may result in disruption of multiple neural signals to, and/or result in local hypoxia of, mucus producing and/or mucosal engorgement elements within the sino-nasal cavity of the patient. Yet still, delivery of the ablation energy may result in therapeutic modulation of postganglionic parasympathetic nerves innervating nasal mucosa at foramina and or microforamina of a palatine bone of the patient. In particular, delivery of the ablation energy causes multiple points of interruption of neural branches extending through foramina and microforamina of palatine bone. Yet still, in some embodiments, delivery of the ablation energy may cause thrombus formation within one or more blood vessels associated with mucus producing and/or mucosal engorgement elements within the nose. The resulting local hypoxia of the mucus producing and/or mucosal engorgement elements may result in decreased mucosal engorgement to thereby increase volumetric flow through a nasal passage of the patient.

Accordingly, the electrical stimulating energy can be applied to the tissue of interest in a highly targeted manner and elicit the desired effect (i.e., neuromodulation, ablation, lesion formation, etc.) to selectively modulate the targeted tissue, while avoiding non-targeted tissue or structures (which may include vital organs or tissues, such as blood vessels) and allowing the surrounding tissue structure to remain healthy for effective wound healing.

In that manner, the present invention solves the problem of causing unnecessary collateral damage to non-targeted tissue during a procedure involving the application of electrotherapeutic stimulation at a target site composed of a variety of tissue types. In particular, the systems and methods are able to, prior to treatment, characterize and identify tissue types and further identify specific levels of energy (i.e., specific target frequencies) to be delivered so as to cause only those intended, targeted tissues to exhibit nearby relaxation phenomena and thus receive the energy for treatment, while non-targeted tissues remain intact and avoid collateral damage.

It should further be noted that, with reference to FIG. 9C, the end effector 114 can continue to sense tissue properties during and/or after treatment. Such sensed data from the end effector 214 can further include feedback data associated with the effect of the stimulating energy at the therapeutic level on targeted tissue at any given location. For example, feedback data (sensed during therapeutic neuromodulation of neural tissue) may be associated with efficacy of ablation of the targeted tissue during and/or after delivery of initial energy from one or more of the plurality of electrodes 244. Accordingly, in certain embodiments, the console 104 (via the controller 107, monitoring system 108, and evaluation/feedback algorithms 110) is configured to process such feedback data to determine if certain properties of the targeted tissue undergoing treatment (i.e., tissue temperature, tissue impedance, etc.) reach predetermined thresholds for irreversible tissue damage.

The electrodes 244 are configured to be independently controlled and activated by the controller 107 (in conjunction with the evaluation/feedback algorithms 110) to thereby deliver energy independent of one another. Accordingly, the controller 107 can tune energy output individually for the one or more electrodes 244 after an initial level of energy has been delivered based, at least in part, on feedback data. For example, once the threshold is reached, the application of therapeutic stimulation energy can be terminated to allow the tissue to remain intact. In other embodiments, if the threshold has not been reached, the controller can maintain, reduce, or increase energy output to a given electrode 244 until such threshold is reached. Accordingly, the energy delivery of any given electrode 244 can automatically be tuned based on an evaluation/feedback algorithm (e.g., the evaluation/feedback algorithm 110 of FIG. 1A) stored on a console (e.g., the console 104 of FIG. 1A) operably coupled to the end effector 214. For example, at least some of the electrodes 244 may have different levels of energy to be delivered at respective positions sufficient to ablate neural tissue at the respective positions based on the feedback data received for the respective locations.

For example, in some embodiments, the controller 107 is configured to tune energy output from each of the plurality of electrodes 244 after an initial level of energy has been delivered based, at least in part, on feedback data received. The feedback data may be associated with efficacy of ablation of the neural tissue at each position during and/or after delivery of initial energy from each of the plurality of electrodes. The feedback data includes one or more properties associated with neural tissue at respective positions. The one or more properties may include, but are not limited to, physiological properties, bioelectric properties, and thermal properties. For example, the active and passive bioelectric properties may include, but are not limited to, complex impedance, resistance, reactance, capacitance, inductance, complex, real and imaginary permittivity, conductivity, nerve firing voltage, nerve firing current, depolarization, hyperpolarization, magnetic field, and induced electromotive force.

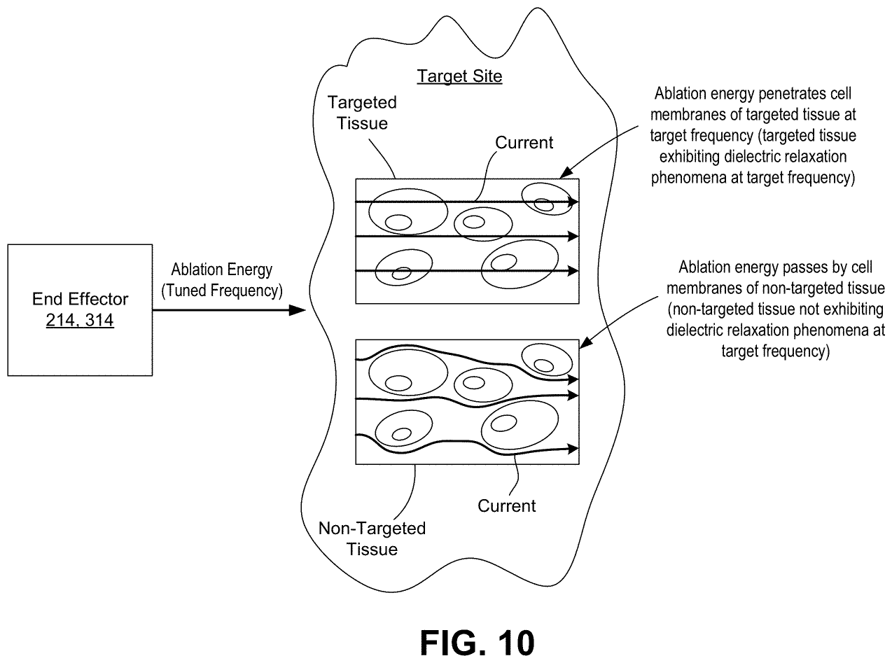

FIG. 11 is a flow diagram illustrating one embodiment of a method 500 for treating a condition. The condition may include, for example, a peripheral neurological condition of a patient. The method 500 includes providing a device comprising an end effector including a plurality of electrodes and a controller operably associated with the device (operation 510). The method 500 further includes positioning the end effector at a target site associated with a patient (operation 520) and receiving, via the controller, data from the device associated with bioelectric properties of one or more tissues at the target site (operation 530).

The bioelectric properties may include, but are not limited to, complex impedance, resistance, reactance, capacitance, inductance, permittivity, conductivity, dielectric properties, muscle or nerve firing voltage, muscle or nerve firing current, depolarization, hyperpolarization, magnetic field, induced electromotive force, and combinations thereof. The dielectric properties may include, for example, at least a complex relative dielectric permittivity. It should be noted that, in some embodiments, a subset of the plurality of electrodes is configured to deliver non-therapeutic stimulating energy at a frequency/waveform to respective positions at the target site to thereby sense the bioelectric properties of the one or more tissues at the target site.

The method 500 further includes processing, via the controller, the data to identify a type of each of the one or more tissues at the target site and further identify a dielectric relaxation pattern for each of the one or more identified tissue types (operation 540).

The processing of the data may include, for example: a) comparing the data received from the device with electric signature data associated with a plurality of known tissue types; and (b) use of (i) a supervised and/or (ii) an unsupervised trained neural network. For example, the controller may be configured to compare the tissue data (i.e., data received from the treatment device associated with tissue at the target site) with profiles of known tissue types stored in a database, for example. Each profile may generally include electric signature data that generally characterizes a known tissue type, including physiological, histological, and bioelectric properties of a known tissue type, including different relaxation phenomena/behavior of the tissue and the specific frequency value at which the tissue exhibits these relaxation phenomena/behavior.

The method 500 further includes determining, via the controller, an ablation pattern to be delivered by one or more of the plurality of electrodes of the end effector based on identified dielectric relaxation patterns. The ablation energy associated with the ablation pattern is at a level sufficient to ablate a targeted tissue and minimize and/or prevent collateral damage to surrounding or adjacent non-targeted tissue at the target site (operation 550). The ablation energy is tuned to a target frequency associated with a relaxation pattern of the targeted tissue. The target frequency comprises a frequency at which the targeted tissue exhibits relaxation phenomena behavior and the non-targeted tissue does not exhibit relaxation phenomena behavior. In particular, delivery of the ablation energy, tuned to the target frequency, penetrates the plasma membrane of one or more cells associated only with the targeted tissue.

In some embodiments, the condition includes a peripheral neurological condition. The peripheral neurological condition may be associated with a nasal condition or a non-nasal condition of the patient. For example, the non-nasal condition may include atrial fibrillation (AF). In some embodiments, the nasal condition may include rhinosinusitis. Accordingly, in some embodiments, the target site is within a sino-nasal cavity of the patient. The delivery of the ablation energy may result in disruption of multiple neural signals to, and/or result in local hypoxia of, mucus producing and/or mucosal engorgement elements within the sino-nasal cavity of the patient. The targeted tissue is proximate or inferior to a sphenopalatine foramen. Yet still, delivery of the ablation energy may result in therapeutic modulation of postganglionic parasympathetic nerves innervating nasal mucosa at foramina and or microforamina of a palatine bone of the patient. In particular, delivery of the ablation energy causes multiple points of interruption of neural branches extending through foramina and microforamina of palatine bone. Yet still, in some embodiments, delivery of the ablation energy may cause thrombus formation within one or more blood vessels associated with mucus producing and/or mucosal engorgement elements within the nose. The resulting local hypoxia of the mucus producing and/or mucosal engorgement elements may result in decreased mucosal engorgement to thereby increase volumetric flow through a nasal passage of the patient.

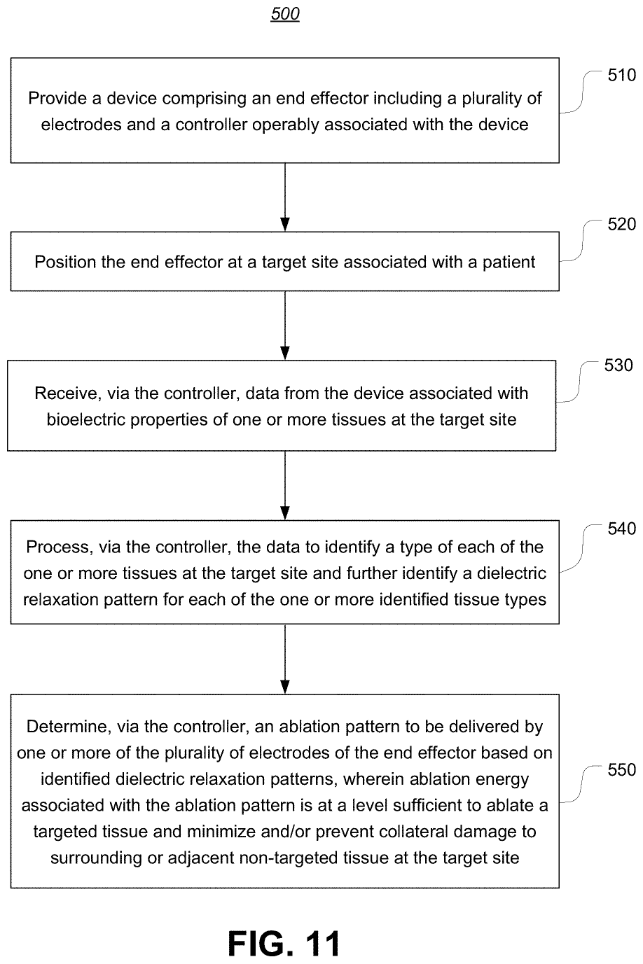

FIG. 12 is a schematic of an exemplary probe/electrode setup for performing some of the methods described herein, most notably for characterizing tissue at a target site by sensing bioelectric properties of tissue, wherein such characterization includes identifying specific types of tissue present and further determining a dielectric relaxation phenomena/behavior pattern for the identified types of tissue.

FIG. 12A is a schematic diagram of one embodiment of a 3-probe/electrode system for sensing bioelectric properties of tissue for subsequent characterization of tissue at a target site, wherein such characterization includes identifying specific types of tissue present and further determining a dielectric relaxation phenomena/behavior pattern for the identified types of tissue.

It should be noted that, while the diagrams of FIGS. 12 and 12A illustrate a 3-probe/electrode system, the systems and methods of the present invention may include any number of probes/electrodes for obtaining bioelectric data from tissue of interest (either targeted tissue or non-targeted tissue) for the purposes of determining dielectric relaxation phenomena/behavior patterns or other properties, as described herein. For example, experimental setups may include the use of 2, 3, 4, or more probes/electrodes.

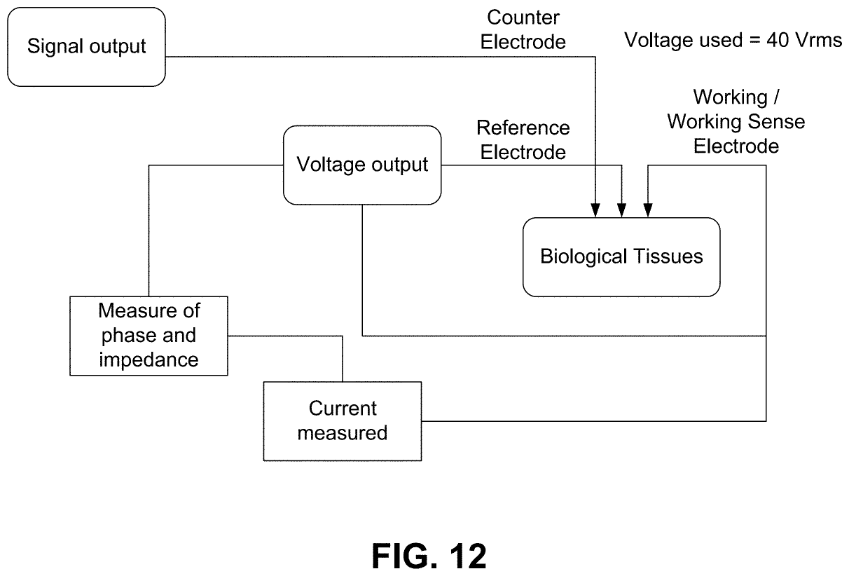

Referring to FIGS. 12 and 12A, the experimental setup includes a 3-electrode assembly (a reference electrode, a counter electrode, and an active working electrode). Such a setup which includes the 3-probe/electrode assembly is used to obtain dielectric properties of various tissue types, wherein such data is described in greater detail herein with reference to FIGS. 13, 14, 15, and 16.

FIGS. 13A and 13B are graphs illustrating dielectric properties of two tissue types (spinal cord and muscle tissues), including the plotting of loss tangent value relative to frequency (FIG. 13A) and the plotting of an imaginary electric modulus relative to frequency (FIG. 13B). As illustrated, the relaxation phenomena/behavior is generally observed around 10 kHz earlier for nervous tissue as compared to muscle tissue.

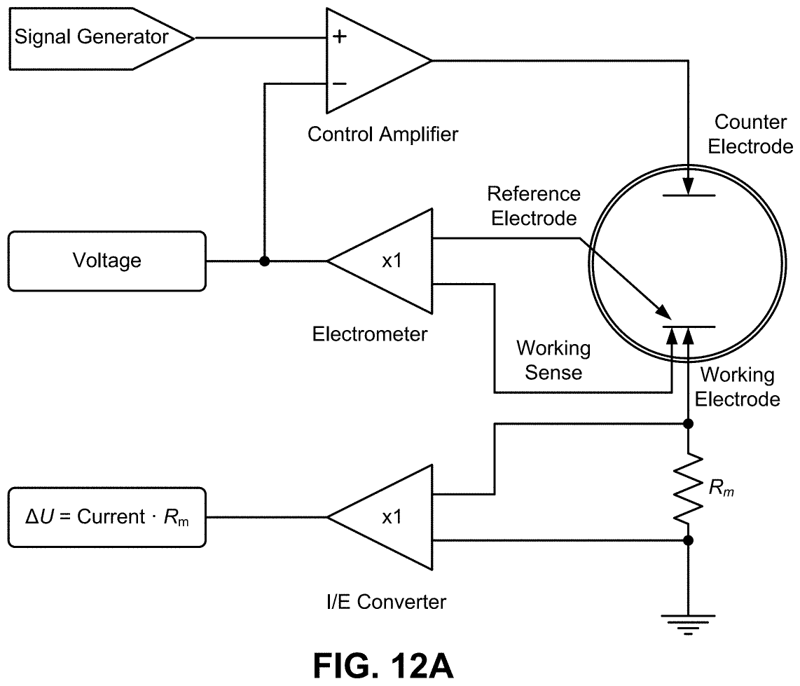

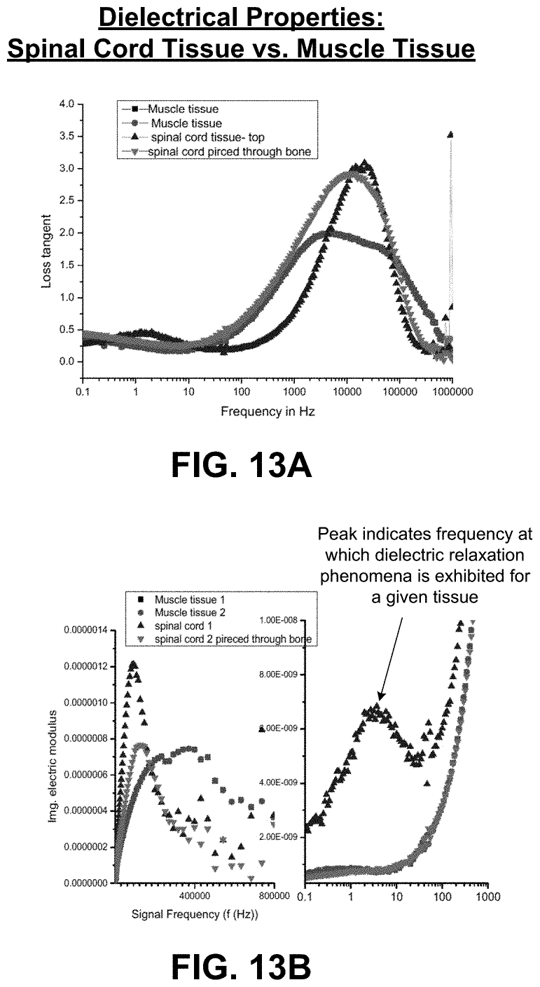

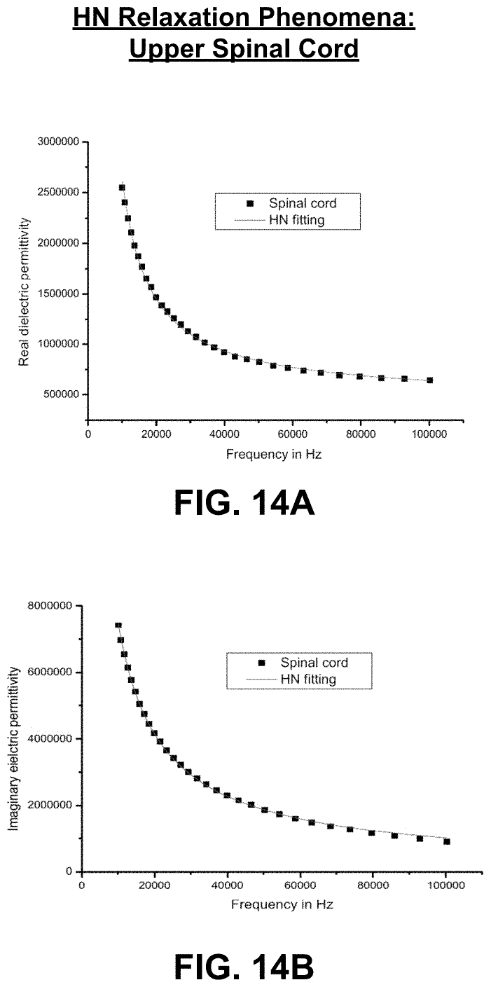

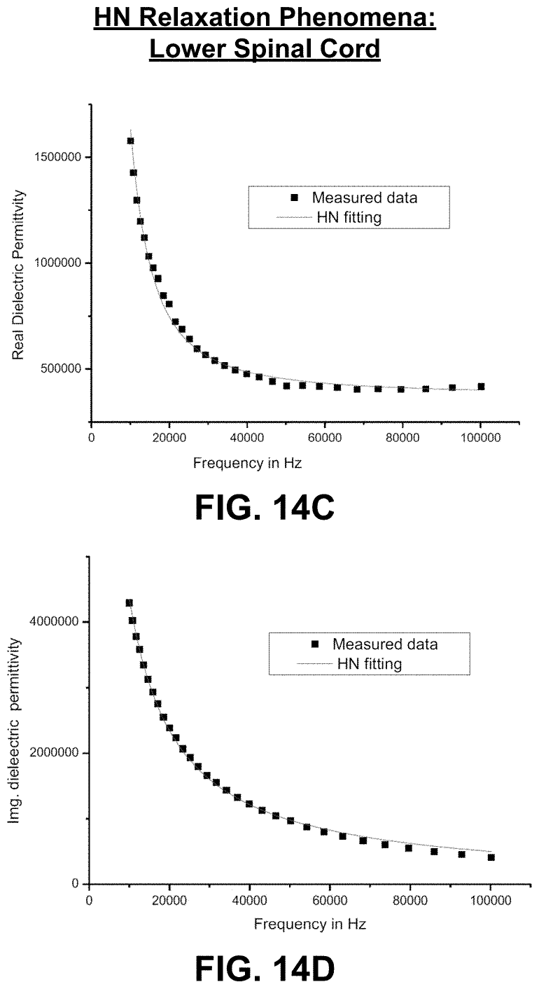

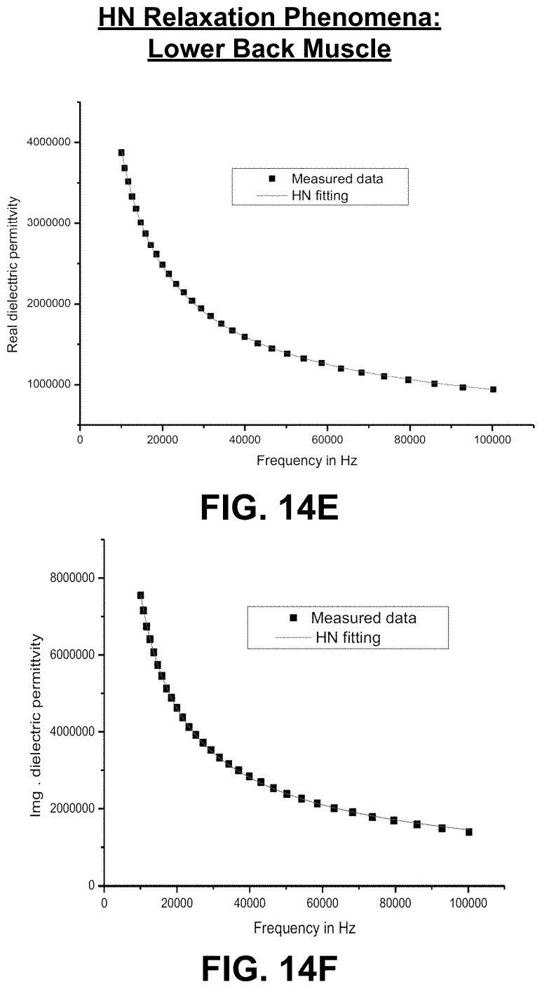

FIGS. 14A-14H are graphs illustrating the plotting of real and imaginary values of the complex relative permittivity (based on the Havriliak-Negami (HN) relaxation phenomena model) relative to frequency for the two tissue types of FIGS. 13A and 13B (spinal cord and muscle tissues).

FIGS. 14A and 14B illustrate plotting of real and imaginary values of the complex relative permittivity relative to frequency for upper spinal cord tissue. FIGS. 14C and 14D illustrate plotting of real and imaginary values of the complex relative permittivity relative to frequency for lower spinal cord tissue. FIGS. 14E and 14F are graphs illustrating the plotting of real and imaginary values of the complex relative permittivity relative to frequency for lower back muscle tissue. FIGS. 14G and 14H are graphs illustrating the plotting of real and imaginary values of the complex relative permittivity relative to frequency for upper back muscle tissue.

Below is a table (Table 1) providing the specific data points for each of the tissues with respect to the real and imaginary dielectric permittivity values or specific HN relaxation parameters obtained for specific tissue types when data obtained from 10 kHz to 80 kHz are trained.

TABLE 1

| Tissue Type | Parameters | Value | Standard Error |
|---|---|---|---|
| Upper Spinal Cord | α | 0 | 0.008 |
| | β | 0.875 | 0.009 |
| | f0 | 1012.70 | 130.20 |
| | ε0-ε∞ | 5.811E7 | 6.12E6 |
| | ε∞ | 431140.35 | 26246.9 |
| Lower Spinal Cord | α | 0.011 | 0.01 |
| | β | 0.98 | 0.01 |
| | f0 | 2629.47 | 208.63 |
| | ε0-ε∞ | 1.72E7 | 1.0E6 |
| | ε∞ | 375317.95 | 25581.28 |
| Lower Back Muscle | α | 0 | 0.004 |
| | β | 0.72 | 0.004 |
| | f0 | 63.23 | 81.80 |
| | ε0-ε∞ | 3.15E8 | 2.89E8 |
| | ε∞ | 241267.91 | 18021.86 |
| Upper Back Muscle | α | 0 | 0.00467 |
| | β | 0.71561 | 0.0044 |
| | f0 | 63.16 | 81.80 |
| | ε0-ε∞ | 3.1E8 | 2.9E8 |
| | ε∞ | 241256.79 | 18021.68 |

It was observed that HN relaxation frequency of upper spinal cord occurs around 1 kHz when two independent equations of real and imaginary dielectric permittivity values are trained for 10 kHz to 80 kHz. It was further observed that HN relaxation of lower spinal cord occurs around 2.6 kHz when two independent equation of real and imaginary dielectric permittivity value are trained for 10 kHz to 80 kHz.

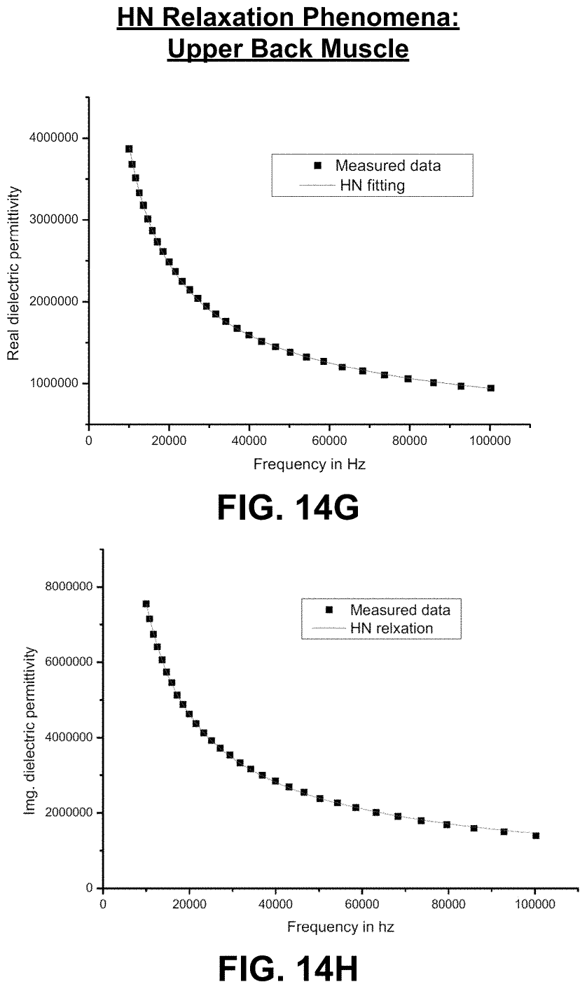

A notable feature of spinal cord is that the conduction behavior is ohmic conductivity when data is train between 10 kHz to 80 kHz as the fitting parameter α is near to 0 and β is near to 1. FIGS. 15A and 15B are graphs illustrating dielectric properties of different portions of a tissue (turbinate tissue), including the plotting of loss tangent value relative to frequency (FIG. 15A) and the plotting of an imaginary electric modulus relative to frequency (FIG. 15B). The different portions of turbinate include an end of the turbinate, center of the turbinate, and portions of the turbinate adjacent to blood vessels. From the data observed (based on peak of tan delta and relaxation), only the center of turbinate appeared to follow the relaxation behavior of nervous tissue, as the center of the turbinate generally includes a bundle of nervous tissue, similar in nature to the lower spinal cord.

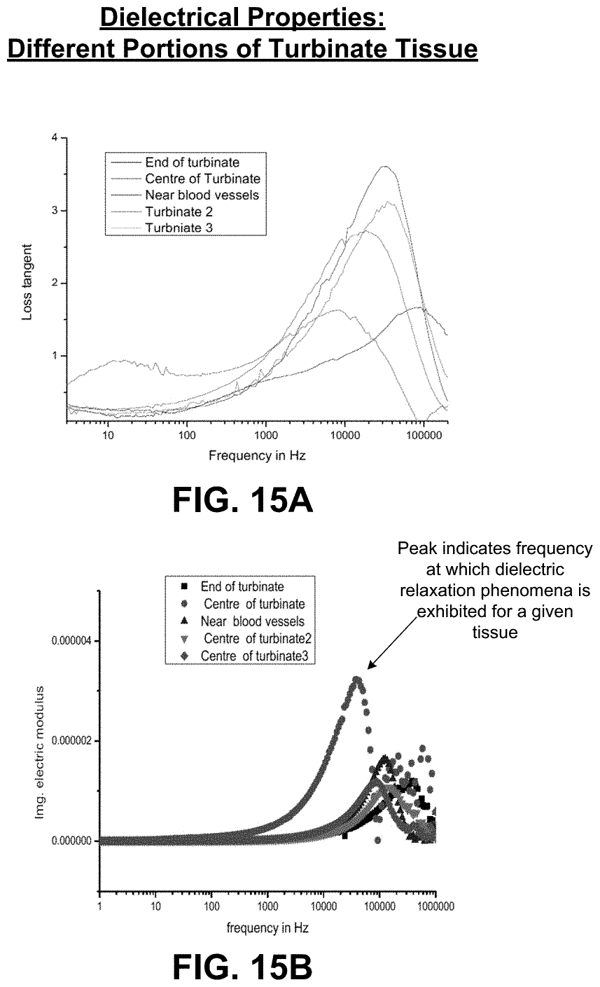

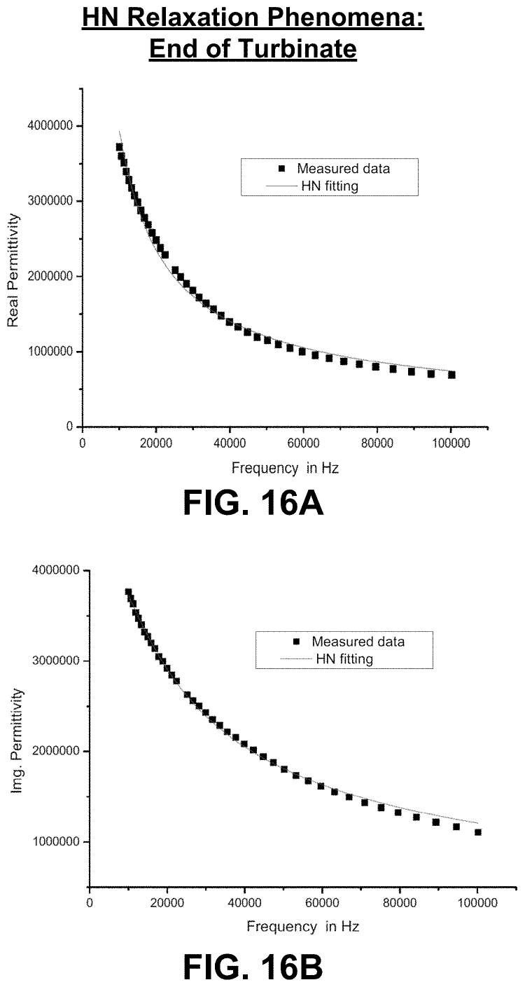

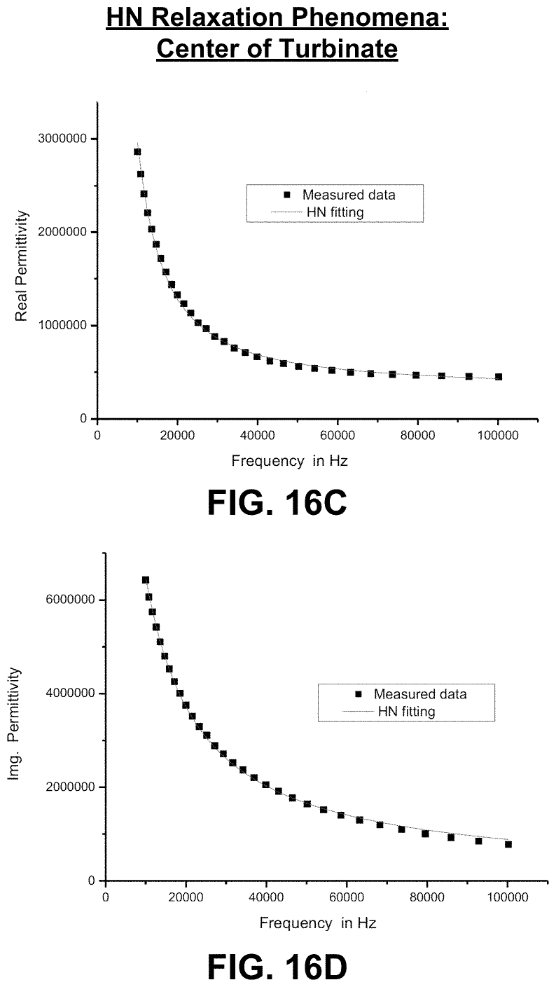

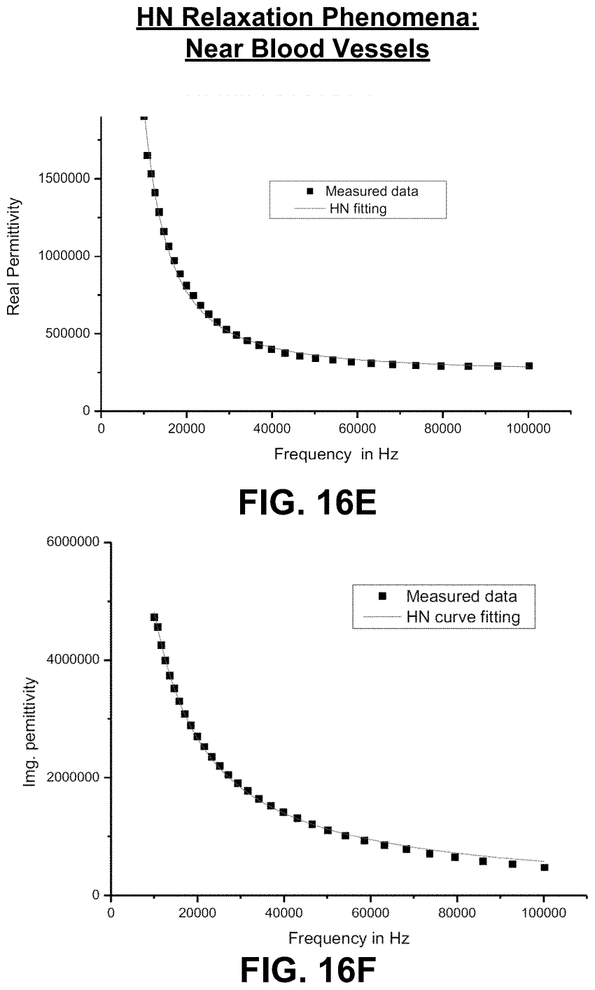

FIGS. 16A-16F are graphs illustrating the plotting of real and imaginary values of the complex relative permittivity (based on the HN relaxation phenomena) relative to frequency for the different portions of the turbinate tissue of FIGS. 15A and 15B.

FIGS. 16A and 16B illustrate plotting of real and imaginary values of the complex relative permittivity relative to frequency for the end of a turbinate tissue. FIGS. 16C and 16D illustrate plotting of real and imaginary values of the complex relative permittivity relative to frequency for the center of the turbinate tissue. FIGS. 16E and 16F are graphs illustrating the plotting of real and imaginary values of the complex relative permittivity relative to frequency for portions of the turbinate tissue near blood vessels.

Below is a table (Table 2) providing the specific data points for each of the tissues with respect to the real and imaginary dielectric permittivity values or specific HN relaxation parameters obtained for specific tissue types when data obtained from 10 kHz to 80 kHz are trained.

TABLE 2

| Tissue Type | Parameters | Value | Standard Error |
|---|---|---|---|
| End of Turbinate | α | 0.082 | 0.009 |
| | β | 0.681 | 0.007 |
| | f0 | 4539.116 | 193.596 |
| | ε0-ε∞ | 9.858E6 | 170687.48 |
| | ε∞ | 1.54761E−10 | — |
| Center of Turbinate | α | 0 | 0.05358 |
| | β | 1 | 0.07788 |
| | f0 | 3290.96 | 859.79 |
| | ε0-ε∞ | 1.60E6 | 298682.17 |
| | ε∞ | 149101.39 | 11446.76 |
| Near Blood Vessels | α | 0.008 | 0.0118 |
| | β | 0.974 | 0.0174 |
| | f0 | 3148.04 | 189.86 |
| | ε0-ε∞ | 1.64E7 | 685597.97 |
| | ε∞ | 244034.20 | 26444.04 |
| Upper Back Muscle | α | 0 | 0.00467 |
| | β | 0.71561 | 0.0044 |
| | f0 | 63.16 | 81.80 |
| | ε0-ε∞ | 3.1E8 | 2.9E8 |
| | ε∞ | 241256.79 | 18021.68 |

Accordingly, the invention solves the problem of causing unnecessary collateral damage to non-targeted tissue during a procedure involving the application of electrotherapeutic stimulation at a target site composed of a variety of tissue types. In particular, the systems and methods are able to, prior to treatment, characterize and identify tissue types and further identify specific levels of energy (i.e., specific target frequencies) to be delivered so as to cause only those intended, targeted tissues to exhibit dielectric relaxation phenomena and thus receive the energy for treatment, while non-targeted tissues remain intact and avoid collateral damage.

The following provides a detailed description of the various capabilities of systems and methods of the present invention, including, but not limited to, neuromodulation monitoring, feedback, and mapping capabilities, which, in turn, allowing for detection of anatomical structures and function, neural identification and mapping, and anatomical mapping, for example.

### Neuromodulation Monitoring, Feedback, and Mapping Capabilities

As previously described, the system 100 includes a console 104 to which the device 102 is to be connected. The console 104 is configured to provide various functions for the device 102, which may include, but is not limited to, controlling, monitoring, supplying, and/or otherwise supporting operation of the device 102. The console 104 can further be configured to generate a selected form and/or magnitude of energy for delivery to tissue or nerves at the target site via the end effector (214, 314), and therefore the console 104 may have different configurations depending on the treatment modality of the device 102. For example, when device 102 is configured for electrode-based, heat-element-based, and/or transducer-based treatment, the console 104 includes an energy generator 106 configured to generate RF energy (e.g., monopolar, bipolar, or multi-polar RF energy), pulsed electrical energy, microwave energy, optical energy, ultrasound energy (e.g., intraluminally-delivered ultrasound and/or HIFU), direct heat energy, radiation (e.g., infrared, visible, and/or gamma radiation), and/or another suitable type of energy. When the device 102 is configured for cryotherapeutic treatment, the console 104 can include a refrigerant reservoir (not shown), and can be configured to supply the device 102 with refrigerant. Similarly, when the device 102 is configured for chemical-based treatment (e.g., drug infusion), the console 104 can include a chemical reservoir (not shown) and can be configured to supply the device 102 with one or more chemicals.

In some embodiments, the console 104 may include a controller 107 communicatively coupled to the device 102. However, in the embodiments described herein, the controller 107 may generally be carried by and provided within the handle 118 of the device 102. The controller 107 is configured to initiate, terminate, and/or adjust operation of one or more electrodes provided by the end effector (214, 314) directly and/or via the console 104. For example, the controller 107 can be configured to execute an automated control algorithm and/or to receive control instructions from an operator (e.g., surgeon or other medical professional or clinician). For example, the controller 107 and/or other components of the console 104 (e.g., processors, memory, etc.) can include a computer-readable medium carrying instructions, which when executed by the controller 107, causes the device 102 to perform certain functions (e.g., apply energy in a specific manner, detect impedance, detect temperature, detect nerve locations or anatomical structures, perform nerve mapping, etc.). A memory includes one or more of various hardware devices for volatile and non-volatile storage, and can include both read-only and writable memory. For example, a memory can comprise random access memory (RAM), CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory.

The console 104 may further be configured to provide feedback to an operator before, during, and/or after a treatment procedure via mapping/evaluation/feedback algorithms 110. For example, the mapping/evaluation/feedback algorithms 110 can be configured to provide information associated with the location of nerves at the treatment site, the location of other anatomical structures (e.g., vessels) at the treatment site, the temperature at the treatment site during monitoring and modulation, and/or the effect of the therapeutic neuromodulation on the nerves at the treatment site. In certain embodiments, the mapping/evaluation/feedback algorithm 110 can include features to confirm efficacy of the treatment and/or enhance the desired performance of the system 100. For example, the mapping/evaluation/feedback algorithm 110, in conjunction with the controller 107 and the end effector (214, 314), can be configured to monitor neural activity and/or temperature at the treatment site during therapy and automatically shut off the energy delivery when the neural activity and/or temperature reaches a predetermined threshold (e.g., a threshold reduction in neural activity, a threshold maximum temperature when applying RF energy, or a threshold minimum temperature when applying cryotherapy). In other embodiments, the mapping/evaluation/feedback algorithm 110, in conjunction with the controller 107, can be configured to automatically terminate treatment after a predetermined maximum time, a predetermined maximum impedance or resistance rise of the targeted tissue (i.e., in comparison to a baseline impedance measurement), a predetermined maximum impedance of the targeted tissue), and/or other threshold values for biomarkers associated with autonomic function. This and other information associated with the operation of the system 100 can be communicated to the operator via a display 112 (e.g., a monitor, touchscreen, user interface, etc.) on the console 104 and/or a separate display (not shown) communicatively coupled to the console 104.

In various embodiments, the end effector (214, 314) and/or other portions of the system 100 can be configured to detect various bioelectric-parameters of the tissue at the target site, and this information can be used by the mapping/evaluation/feedback algorithms 110 to determine the anatomy at the target site (e.g., tissue types, tissue locations, vasculature, bone structures, foramen, sinuses, etc.), locate neural tissue, differentiate between different types of neural tissue, map the anatomical and/or neural structure at the target site, and/or identify neuromodulation patterns of the end effector (214, 314) with respect to the patient's anatomy. For example, the end effector (214, 314) can be used to detect resistance, complex electrical impedance, dielectric properties, temperature, and/or other properties that indicate the presence of neural fibers and/or other anatomical structures in the target region. In certain embodiments, the end effector (214, 314), together with the mapping/evaluation/feedback algorithms 110, can be used to determine resistance (rather than impedance) of the tissue (i.e., the load) to more accurately identify the characteristics of the tissue. The mapping/evaluation/feedback algorithms 110 can determine resistance of the tissue by detecting the actual power and current of the load (e.g., via the electrodes (244, 336)).

In some embodiments, the system 100 provides resistance measurements with a high degree of accuracy and a very high degree of precision, such as precision measurements to the hundredths of an Ohm (e.g., 0.01Ω) for the range of 1-2000Ω. The high degree of resistance detection accuracy provided by the system 100 allows for the detection sub-microscale structures and events, including the firing of neural tissue, differences between neural tissue and other anatomical structures (e.g., blood vessels), and event different types of neural tissue. This information can be analyzed by the mapping/evaluation/feedback algorithms and/or the controller 107 and communicated to the operator via a high resolution spatial grid (e.g., on the display 112) and/or other type of display to identify neural tissue and other anatomy at the treatment site and/or indicate predicted neuromodulation regions based on the ablation pattern with respect to the mapped anatomy.

As previously described, in certain embodiments, each electrode (244, 336) can be operated independently of the other electrodes (244, 336). For example, each electrode can be individually activated and the polarity and amplitude of each electrode can be selected by an operator or a control algorithm executed by the controller 107. The selective independent control of the electrodes (244, 336) allows the end effector (214, 314) to detect information and deliver RF energy to highly customized regions. For example, a select portion of the electrodes (244, 336) can be activated to target specific neural fibers in a specific region while the other electrodes (244, 336) remain inactive. In certain embodiments, for example, electrodes (244, 336) may be activated across the portion of a strut that is adjacent to tissue at the target site, and the electrodes (244, 336) that are not proximate to the target tissue can remain inactive to avoid applying energy to non-target tissue. In addition, the electrodes (244, 336) can be individually activated to stimulate or therapeutically modulate certain regions in a specific pattern at different times (e.g., via multiplexing), which facilitates detection of anatomical parameters across a zone of interest and/or regulated therapeutic neuromodulation.

The electrodes (244, 336) can be electrically coupled to the energy generator 106 via wires (not shown) that extend from the electrodes (244, 336), through the shaft 116, and to the energy generator 106. When each of the electrodes (244, 336) is independently controlled, each electrode (244, 336) couples to a corresponding wire that extends through the shaft 116. This allows each electrode (244, 336) to be independently activated for stimulation or neuromodulation to provide precise ablation patterns and/or individually detected via the console 104 to provide information specific to each electrode (244, 336) for neural or anatomical detection and mapping. In other embodiments, multiple electrodes (244, 336) can be controlled together and, therefore, multiple electrodes (244, 336) can be electrically coupled to the same wire extending through the shaft 116. The energy generator 16 and/or components (e.g., a control module) operably coupled thereto can include custom algorithms to control the activation of the electrodes (244, 336). For example, the RF generator can deliver RF power at about 200-100 W to the electrodes (244, 336), and do so while activating the electrodes (244, 336) in a predetermined pattern selected based on the position of the end effector (214, 314) relative to the treatment site and/or the identified locations of the target nerves. In other embodiments, the energy generator 106 delivers power at lower levels (e.g., less than 1 W, 1-5 W, 5-15 W, 15-50 W, 50-150 W, etc.) for stimulation and/or higher power levels. For example, the energy generator 106 can be configured to delivery stimulating energy pulses of 1-3 W via the electrodes (244, 336) to stimulate specific targets in the tissue.

As previously described, the end effector (214, 314) can further include one or more temperature sensors disposed on the struts and/or other portions of the end effector (214, 314) and electrically coupled to the console 104 via wires (not shown) that extend through the shaft 116. In various embodiments, the temperature sensors can be positioned proximate to the electrodes (244, 336) to detect the temperature at the interface between tissue at the target site and the electrodes (244, 336). In other embodiments, the temperature sensors can penetrate the tissue at the target site (e.g., a penetrating thermocouple) to detect the temperature at a depth within the tissue. The temperature measurements can provide the operator or the system with feedback regarding the effect of the therapeutic neuromodulation on the tissue. For example, in certain embodiments the operator may wish to prevent or reduce damage to the tissue at the treatment site, and therefore the temperature sensors can be used to determine if the tissue temperature reaches a predetermined threshold for irreversible tissue damage. Once the threshold is reached, the application of therapeutic neuromodulation energy can be terminated to allow the tissue to remain intact and avoid significant tissue sloughing during wound healing. In certain embodiments, the energy delivery can automatically terminate based on the mapping/evaluation/feedback algorithm 110 stored on the console 104 operably coupled to the temperature sensors.

In certain embodiments, the system 100 can determine the locations and/or morphology of neural tissue and/or other anatomical structures before therapy such that the therapeutic neuromodulation can be applied to precise regions including target neural tissue, while avoiding negative effects on non-target structures, such as blood vessels. As described in further detail below, the system 100 can detect various bioelectrical parameters in an interest zone to determine the location and morphology of various neural tissue (e.g., different types of neural tissue, neuronal directionality, etc.) and/or other tissue (e.g., glandular structures, vessels, bony regions, etc.). In some embodiments, the system 100 is configured to measure bioelectric potential. To do so, one or more of the electrodes (244, 336) is placed in contact with an epithelial surface at a region of interest (e.g., a treatment site). Electrical stimuli (e.g., constant or pulsed currents at one or more frequencies) are applied to the tissue by one or more electrodes (244, 336) at or near the treatment site, and the voltage and/or current differences at various different frequencies between various pairs of electrodes (244, 336) of the end effector (214, 314) may be measured to produce a spectral profile or map of the detected bioelectric potential, which can be used to identify different types of tissues (e.g., vessels, neural tissue, and/or other types of tissue) in the region of interest. For example, current (i.e., direct or alternating current) can be applied to a pair of electrodes (244, 336) adjacent to each other and the resultant voltages and/or currents between other pairs of adjacent electrodes (244, 336) are measured. It will be appreciated that the current injection electrodes (244, 336) and measurement electrodes (244, 336) need not be adjacent, and that modifying the spacing between the two current injection electrodes (244, 336) can affect the depth of the recorded signals. For example, closely-spaced current injection electrodes (244, 336) provided recorded signals associated with tissue deeper from the surface of the tissue than further spaced apart current injection electrodes (244, 336) that provide recorded signals associated with tissue at shallower depths. Recordings from electrode pairs with different spacings may be merged to provide additional information on depth and localization of anatomical structures.

Further, complex impedance and/or resistance measurements of the tissue at the region of interest can be detected directly from current-voltage data provided by the bioelectric measurements while differing levels of frequency currents are applied to the tissue (e.g., via the end effector (214, 314)), and this information can be used to map the neural and anatomical structures by the use of frequency differentiation reconstruction. Applying the stimuli at different frequencies will target different stratified layers or cellular bodies or clusters. At high signal frequencies (e.g., electrical injection or stimulation), for example, cell membranes of the neural tissue do not impede current flow, and the current passes directly through the cell membranes. In this case, the resultant measurement (e.g., impedance, resistance, capacitance, and/or induction) is a function of the intracellular and extracellular tissue and liquids, ions, proteins and polysaccharides. At low signal frequencies, the membranes impede current flow to provide different defining characteristics of the tissues, such as the shapes and morphologies of the cells or cell densities or cell spacing. The stimulation frequencies can be in the megahertz range, in the kilohertz range (e.g., 400-500 kHz, 450-480 kHz, etc.), and/or other frequencies attuned to the tissue being stimulated and the characteristics of the device being used. The detected complex impedance or resistances levels from the zone of interest can be displayed to the user (e.g., via the display 112) to visualize certain structures based on the stimulus frequency.

Further, the inherent morphology and composition of the anatomical structures in a given region or zone of the patient react differently to different frequencies and, therefore, specific frequencies can be selected to identify very specific structures. For example, the morphology or composition of targeted structures for anatomical mapping may depend on whether the cells of tissue or other structure are membranonic, stratified, and/or annular. In various embodiments, the applied stimulation signals can have predetermined frequencies attuned to specific neural tissue, such as the level of myelination and/or morphology of the myelination. For example, second axonal parasympathetic structures are poorly myelinated than sympathetic nerves or other structures and, therefore, will have a distinguishable response (e.g., complex impedance, resistance, etc.) with respect to a selected frequency than sympathetic nerves. Accordingly, applying signals with different frequencies to the target site can distinguish the targeted parasympathetic nerves from the non-targeted sensory nerves, and therefore provide highly specific target sites for neural mapping before or after therapy and/or neural evaluation post-therapy. In some embodiments, the neural and/or anatomical mapping includes measuring data at a region of interest with at least two different frequencies to identify certain anatomical structures such that the measurements are taken first based on a response to an injection signal having a first frequency and then again based on an injection signal having a second frequency different from the first. For example, there are two frequencies at which hypertrophied (i.e., disease-state characteristics) sub-mucosal targets have a different electrical conductivity or permittivity compared to “normal” (i.e., healthy) tissue. Complex conductivity may be determined based on one or more measured physiological parameters (e.g., complex impedance, resistance, dielectric measurements, dipole measurements, etc.) and/or observance of one or more confidently known attributes or signatures. Furthermore, the system 100 can also apply neuromodulation energy via the electrodes (244, 336) at one or more predetermined frequencies attuned to a target neural structure to provide highly targeted ablation of the selected neural structure associated with the frequency(ies). This highly targeted neuromodulation also reduces the collateral effects of neuromodulation therapy to non-target sites/structures (e.g., blood vessels) because the targeted signal (having a frequency tuned to a target neural structure) will not have the same modulating effects on the non-target structures.

Accordingly, bioelectric properties, such as complex impedance and resistance, can be used by the system 100 before, during, and/or after neuromodulation therapy to guide one or more treatment parameters. For example, before, during, and/or after treatment, impedance or resistance measurements may be used to confirm and/or detect contact between one or more electrodes (244, 336) and the adjacent tissue. The impedance or resistance measurements can also be used to detect whether the electrodes (244, 336) are placed appropriately with respect to the targeted tissue type by determining whether the recorded spectra have a shape consistent with the expected tissue types and/or whether serially collected spectra were reproducible. In some embodiments, impedance or resistance measurements may be used to identify a boundary for the treatment zone (e.g., specific neural tissue that are to be disrupted), anatomical landmarks, anatomical structures to avoid (e.g., vascular structures or neural tissue that should not be disrupted), and other aspects of delivering energy to tissue.

The bioelectric information can be used to produce a spectral profile or map of the different anatomical features tissues at the target site, and the anatomical mapping can be visualized in a 3D or 2D image via the display 112 and/or other user interface to guide the selection of a suitable treatment site. This neural and anatomical mapping allows the system 100 to accurately detect and therapeutically modulate the postganglionic parasympathetic neural fibers that innervate the mucosa at numerous neural entrance points within a given zone or region of a patient. Further, because there are not any clear anatomical markers denoting the location of the SPF, accessory foramen, and microforamina, the neural mapping allows the operator to identify and therapeutically modulate nerves that would otherwise be unidentifiable without intricate dissection of the mucosa. In addition, anatomical mapping also allows the clinician to identify certain structures that the clinician may wish to avoid during therapeutic neural modulation (e.g., certain arteries). The neural and anatomical bioelectric properties detected by the system 100 can also be used during and after treatment to determine the real-time effect of the therapeutic neuromodulation on the treatment site. For example, the mapping/evaluation/feedback algorithms 110 can also compare the detected neural locations and/or activity before and after therapeutic neuromodulation, and compare the change in neural activity to a predetermined threshold to assess whether the application of therapeutic neuromodulation was effective across the treatment site.

In various embodiments, the system 100 can also be configured to map the expected therapeutic modulation patterns of the electrodes (244, 336) at specific temperatures and, in certain embodiments, take into account tissue properties based on the anatomical mapping of the target site. For example, the system 100 can be configured to map the ablation pattern of a specific electrode ablation pattern at the 45° C. isotherm, the 55° C. isotherm, the 65° C. isotherm, and/or other temperature/ranges (e.g., temperatures ranging from 45° C. to 70° C. or higher) depending on the target site and/or structure.

The system 100 may provide, via the display 112, three-dimensional views of such projected ablation patterns of the electrodes (244, 336) of the end effector (214, 314). The ablation pattern mapping may define a region of influence that each electrode (244, 336) has on the surrounding tissue. The region of influence may correspond to the region of tissue that would be exposed to therapeutically modulating energy based on a defined electrode activation pattern (i.e., one, two, three, four, or more electrodes on any given strut). In other words, the ablation pattern mapping can be used to illustrate the ablation pattern of any number of electrodes (244, 336), any geometry of the electrode layout, and/or any ablation activation protocol (e.g., pulsed activation, multi-polar/sequential activation, etc.).

In some embodiments, the ablation pattern may be configured such that each electrode (244, 336) has a region of influence surrounding only the individual electrode (244, 336) (i.e., a “dot” pattern). In other embodiments, the ablation pattern may be such that two or more electrodes (244, 336) may link together to form a sub-grouped regions of influence that define peanut-like or linear shapes between two or more electrodes (244, 336). In further embodiments, the ablation pattern can result in a more expansive or contiguous pattern in which the region of influence extends along multiple electrodes (244, 336) (e.g., along each strut). In still further embodiments, the ablation pattern may result in different regions of influence depending upon the electrode activation pattern, phase angle, target temperature, pulse duration, device structure, and/or other treatment parameters. The three-dimensional views of the ablation patterns can be output to the display 112 and/or other user interfaces to allow the clinician to visualize the changing regions of influence based on different durations of energy application, different electrode activation sequences (e.g., multiplexing), different pulse sequences, different temperature isotherms, and/or other treatment parameters. This information can be used to determine the appropriate ablation algorithm for a patient's specific anatomy. In other embodiments, the three-dimensional visualization of the regions of influence can be used to illustrate the regions from which the electrodes (244, 336) detect data when measuring bioelectrical properties for anatomical mapping. In this embodiment, the three dimensional visualization can be used to determine which electrode activation pattern should be used to determine the desired properties (e.g., impedance, resistance, etc.) in the desired area. In certain embodiments, it may be better to use dot assessments, whereas in other embodiments it may be more appropriate to detect information from linear or larger contiguous regions.

In some embodiments, the mapped ablation pattern is superimposed on the anatomical mapping to identify what structures (e.g., neural tissue, vessels, etc.) will be therapeutically modulated or otherwise affected by the therapy. An image may be provided to the surgeon which includes a digital illustration of a predicted or planned neuromodulation zone in relation to previously identified anatomical structures in a zone of interest. For example, the illustration may show numerous neural tissue and, based on the predicted neuromodulation zone, identifies which neural tissue are expected to be therapeutically modulated. The expected therapeutically modulated neural tissue may be shaded to differentiate them from the non-affected neural tissue. In other embodiments, the expected therapeutically modulated neural tissue can be differentiated from the non-affected neural tissue using different colors and/or other indicators. In further embodiments, the predicted neuromodulation zone and surrounding anatomy (based on anatomical mapping) can be shown in a three dimensional view (and/or include different visualization features (e.g., color-coding to identify certain anatomical structures, bioelectric properties of the target tissue, etc.). The combined predicted ablation pattern and anatomical mapping can be output to the display 112 and/or other user interfaces to allow the clinician to select the appropriate ablation algorithm for a patient's specific anatomy.

The imaging provided by the system 100 allows the clinician to visualize the ablation pattern before therapy and adjust the ablation pattern to target specific anatomical structures while avoiding others to prevent collateral effects. For example, the clinician can select a treatment pattern to avoid blood vessels, thereby reducing exposure of the vessel to the therapeutic neuromodulation energy. This reduces the risk of damaging or rupturing vessels and, therefore, prevents immediate or latent bleeding. Further, the selective energy application provided by the neural mapping reduces collateral effects of the therapeutic neuromodulation, such as tissue sloughing off during wound healing (e.g., 1-3 weeks post ablation), thereby reducing the aspiration risk associated with the neuromodulation procedure.

The system 100 can be further configured to apply neuromodulation energy (via the electrodes (244, 336)) at specific frequencies attuned to the target neural structure and, therefore, specifically target desired neural tissue over non-target structures. For example, the specific neuromodulation frequencies can correspond to the frequencies identified as corresponding to the target structure during neural mapping. As described above, the inherent morphology and composition of the anatomical structures react differently to different frequencies. Thus, frequency-tuned neuromodulation energy tailored to a target structure does not have the same modulating effects on non-target structures. More specifically, applying the neuromodulation energy at the target-specific frequency causes ionic agitation in the target neural structure, leading to differentials in osmotic potentials of the targeted neural tissue and dynamic changes in neuronal membronic potentials (resulting from the difference in intra-cellular and extra-cellular fluidic pressure). This causes degeneration, possibly resulting in vacuolar degeneration and, eventually, necrosis at the target neural structure, but is not expected to functionally affect at least some non-target structures (e.g., blood vessels). Accordingly, the system 100 can use the neural-structure specific frequencies to both (1) identify the locations of target neural tissue to plan electrode ablation configurations (e.g., electrode geometry and/or activation pattern) that specifically focus the neuromodulation on the target neural structure; and (2) apply the neuromodulation energy at the characteristic neural frequencies to selectively ablate the neural tissue responsive to the characteristic neural frequencies. For example, the end effector (214, 314) of the system 100 may selectively stimulate and/or modulate parasympathetic fibers, sympathetic fibers, sensory fibers, alpha/beta/delta fibers, C-fibers, anoxic terminals of one or more of the foregoing, insulated over non-insulated fibers (regions with fibers), and/or other neural tissue. In some embodiments, the system 100 may also selectively target specific cells or cellular regions during anatomical mapping and/or therapeutic modulation, such as smooth muscle cells, sub-mucosal glands, goblet cells, and stratified cellular regions within a given tissue type. Therefore, the system 100 provides highly selective neuromodulation therapy specific to targeted neural tissue, and reduces the collateral effects of neuromodulation therapy to non-target structures (e.g., blood vessels).

The present disclosure provides a method of anatomical mapping and therapeutic neuromodulation. The method includes expanding an end effector (i.e., end effector (214, 314)) at a zone of interest (“interest zone”). For example, the end effector (214, 314) can be expanded such that at least some of the electrodes (244, 336) are placed in contact with tissue at the interest zone. The expanded device can then take bioelectric measurements via the electrodes (244, 336) and/or other sensors to ensure that the desired electrodes are in proper contact with the tissue at the interest zone. In some embodiments, for example, the system 100 detects the impedance and/or resistance across pairs of the electrodes (244, 336) to confirm that the desired electrodes have appropriate surface contact with the tissue and that all of the electrodes are (244, 336) functioning properly.

The method continues by optionally applying an electrical stimulus to the tissue, and detecting bioelectric properties of the tissue to establish baseline norms of the tissue. For example, the method can include measuring resistance, complex impedance, current, voltage, nerve firing rate, neuromagnetic field, muscular activation, and/or other parameters that are indicative of the location and/or function of neural tissue and/or other anatomical structures (e.g., glandular structures, blood vessels, etc.). In some embodiments, the electrodes (244, 336) send one or more stimulation signals (e.g., pulsed signals or constant signals) to the interest zone to stimulate neural activity and initiate action potentials. The stimulation signal can have a frequency attuned to a specific target structure (e.g., a specific neural structure, a glandular structure, a vessel) that allows for identification of the location of the specific target structure. The specific frequency of the stimulation signal is a function of the host permeability and, therefore, applying the unique frequency alters the tissue attenuation and the depth into the tissue the RF energy will penetrate. For example, lower frequencies typically penetrate deeper into the tissue than higher frequencies.

Pairs of the non-stimulating electrodes (244, 336) of the end effector (214, 314) can then detect one or more bioelectric properties of the tissue that occur in response to the stimulus, such as impedance or resistance. For example, an array of electrodes (e.g., the electrodes (244, 336)) can be selectively paired together in a desired pattern (e.g., multiplexing the electrodes (244, 336)) to detect the bioelectric properties at desired depths and/or across desired regions to provide a high level of spatial awareness at the interest zone. In certain embodiments, the electrodes (244, 336) can be paired together in a time-sequenced manner according to an algorithm (e.g., provided by the mapping/evaluation/feedback algorithms 110). In various embodiments, stimuli can be injected into the tissue at two or more different frequencies, and the resultant bioelectric responses (e.g., action potentials) in response to each of the injected frequencies can be detected via various pairs of the electrodes (244, 336). For example, an anatomical or neural mapping algorithm can cause the end effector (214, 314) to deliver pulsed RF energy at specific frequencies between different pairs of the electrodes (244, 336) and the resultant bioelectric response can be recorded in a time sequenced rotation until the desired interest zone is adequately mapped (i.e., “multiplexing”). For example, the end effector (214, 314) can deliver stimulation energy at a first frequency via adjacent pairs of the electrodes (244, 336) for a predetermined time period (e.g., 1-50 milliseconds), and the resultant bioelectric activity (e.g., resistance) can be detected via one or more other pairs of electrodes (244, 336) (e.g., spaced apart from each other to reach varying depths within the tissue). The end effector (214, 314) can then apply stimulation energy at a second frequency different from the first frequency, and the resultant bioelectric activity can be detected via the other electrodes. This can continue when the interest zone has been adequately mapped at the desired frequencies. As described in further detail below, in some embodiments the baseline tissue bioelectric properties (e.g., nerve firing rate) are detected using static detection methods (without the injection of a stimulation signal).

After detecting the baseline bioelectric properties, the information can be used to map anatomical structures and/or functions at the interest zone. For example, the bioelectric properties detected by the electrodes (244, 336) can be amazed via the mapping/evaluation/feedback algorithms 110, and an anatomical map can be output to a user via the display 112. In some embodiments, complex impedance, dielectric, or resistance measurements can be used to map parasympathetic nerves and, optionally, identify neural tissue in a diseased state of hyperactivity. The bioelectric properties can also be used to map other non-target structures and the general anatomy, such as blood vessels, bone, and/or glandular structures. The anatomical locations can be provided to a user (e.g., on the display 112) as a two-dimensional map (e.g., illustrating relative intensities, illustrating specific sites of potential target structures) and/or as a three-dimensional image. This information can be used to differentiate structures on a submicron, cellular level and identify very specific target structures (e.g., hyperactive parasympathetic nerves). The method can also predict the ablation patterns of the end effector (214, 314) based on different electrode neuromodulation protocol and, optionally, superimpose the predicted neuromodulation patterns onto the mapped anatomy to indicate to the user which anatomical structures will be affected by a specific neuromodulation protocol. For example, when the predicted neuromodulation pattern is displayed in relation to the mapped anatomy, a clinician can determine whether target structures will be appropriately ablated and whether non-target structures (e.g., blood vessels) will be undesirably exposed to the therapeutic neuromodulation energy. Thus, the method can be used for planning neuromodulation therapy to locate very specific target structures, avoid non-target structures, and select electrode neuromodulation protocols.

Once the target structure is located and a desired electrode neuromodulation protocol has been selected, the method continues by applying therapeutic neuromodulation to the target structure. The neuromodulation energy can be applied to the tissue in a highly targeted manner that forms micro-lesions to selectively modulate the target structure, while avoiding non-targeted blood vessels and allowing the surrounding tissue structure to remain healthy for effective wound healing. In some embodiments, the neuromodulation energy can be applied in a pulsed manner, allowing the tissue to cool between modulation pulses to ensure appropriate modulation without undesirably affecting non-target tissue. In some embodiments, the neuromodulation algorithm can deliver pulsed RF energy between different pairs of the electrodes (244, 336) in a time sequenced rotation until neuromodulation is predicted to be complete (i.e., “multiplexing”). For example, the end effector (214, 314) can deliver neuromodulation energy (e.g., having a power of 5-10 W (e.g., 7 W, 8 W, 9 W) and a current of about 50-100 mA) via adjacent pairs of the electrodes (244, 336) until at least one of the following conditions is met: (a) load resistance reaches a predefined maximum resistance (e.g., 350Ω); (b) a thermocouple temperature associated with the electrode pair reaches a predefined maximum temperature (e.g., 80° C.); or (c) a predetermined time period has elapsed (e.g., 10 seconds). After the predetermined conditions are met, the end effector (214, 314) can move to the next pair of electrodes in the sequence, and the neuromodulation algorithm can terminate when all of the load resistances of the individual pairs of electrodes is at or above a predetermined threshold (e.g., 100Ω). In various embodiments, the RF energy can be applied at a predetermined frequency (e.g., 450-500 kHz) and is expected to initiate ionic agitation of the specific target structure, while avoiding functional disruption of non-target structures.

During and/or after neuromodulation therapy, the method continues by detecting and, optionally, mapping the post-therapy bioelectric properties of the target site. This can be performed in a similar manner as described above. The post-therapy evaluation can indicate if the target structures (e.g., hyperactive parasympathetic nerves) were adequately modulated or ablated. If the target structures are not adequately modulated (i.e., if neural activity is still detected in the target structure and/or the neural activity has not decreased), the method can continue by again applying therapeutic neuromodulation to the target. If the target structures were adequately ablated, the neuromodulation procedure can be completed.

### Detection of Anatomical Structures and Function

Various embodiments of the present technology can include features that measure bio-electric, dielectric, and/or other properties of tissue at target sites to determine the presence, location, and/or activity of neural tissue and other anatomical structures and, optionally, map the locations of the detected neural tissue and/or other anatomical structures. For example, the present technology can be used to detect glandular structures and, optionally, their mucoserous functions and/or other functions. The present technology can also be configured to detect vascular structures (e.g., arteries) and, optionally, their arterial functions, volumetric pressures, and/or other functions. The mapping features discussed below can be incorporated into any the system 100 and/or any other devices disclosed herein to provide an accurate depiction of nerves at the target site.

Neural and/or anatomical detection can occur (a) before the application of a therapeutic neuromodulation energy to determine the presence or location of neural tissue and other anatomical structures (e.g., blood vessels, glands, etc.) at the target site and/or record baseline levels of neural activity; (b) during therapeutic neuromodulation to determine the real-time effect of the energy application on the neural fibers at the treatment site; and/or (c) after therapeutic neuromodulation to confirm the efficacy of the treatment on the targeted structures (e.g., nerves glands, etc.). This allows for the identification of very specific anatomical structures (even to the micro-scale or cellular level) and, therefore, provides for highly targeted neuromodulation. This enhances the efficacy and efficiency of the neuromodulation therapy. In addition, the anatomical mapping reduces the collateral effects of neuromodulation therapy to non-target sites. Accordingly, the targeted neuromodulation inhibits damage or rupture of blood vessels (i.e., inhibits undesired bleeding) and collateral damage to tissue that may be of concern during wound healing (e.g., when damaged tissue sloughs off).

In certain embodiments, the systems disclosed herein can use bioelectric measurements, such as impedance, resistance, voltage, current density, and/or other parameters (e.g., temperature) to determine the anatomy, in particular the neural, glandular, and vascular anatomy, at the target site. The bioelectric properties can be detected after the transmission of a stimulus (e.g., an electrical stimulus, such as RF energy delivered via the electrodes (244, 336); i.e., “dynamic” detection) and/or without the transmission of a stimulus (i.e., “static” detection).

Dynamic measurements include various embodiments to excite and/or detect primary or secondary effects of neural activation and/or propagation. Such dynamic embodiments involve the heightened states of neural activation and propagation and use this dynamic measurement for nerve location and functional identification relative to the neighboring tissue types. For example, a method of dynamic detection can include: (1) delivering stimulation energy to a treatment site via a treatment device (e.g., the end effector) to excite parasympathetic nerves at the treatment site; (2) measuring one or more physiological parameters (e.g., resistance, impedance, etc.) at the treatment site via a measuring/sensing array of the treatment device (e.g., the electrodes (244, 336)); (4) based on the measurements, identifying the relative presence and position of parasympathetic nerves at the treatment site; and (5) delivering ablation energy to the identified parasympathetic nerves to block the detected para-sympathetic nerves.

Static measurements include various embodiments associated with specific native properties of the stratified or cellular composition at or near the treatment site. The static embodiments are directed to inherent biologic and electrical properties of tissue types at or near the treatment site, the stratified or cellular compositions at or near the treatment site, and contrasting both foregoing measurements with tissue types adjacent the treatment site (and that are not targeted for neuromodulation). This information can be used to localize specific targets (e.g., parasympathetic fibers) and non-targets (e.g., vessels, sensory nerves, etc.). For example, a method of static detection can include: (1) before ablation, utilizing a measuring/sensing array of a treatment device (e.g., the electrodes (244, 336)) to determine one or more baseline physiological parameters; (2) geometrically identifying inherent tissue properties within a region of interest based on the measured physiological parameters (e.g., resistance, impedance, etc.); (3) delivering ablation energy to one or more nerves within the region of via treatment device interest; (4) during the delivery of the ablation energy, determining one or more mid-procedure physiological parameters via the measuring/sensing array; and (5) after the delivery of ablation energy, determining one or more post-procedure physiological parameters via the measurement/sensing array to determine the effectiveness of the delivery of the ablation energy on blocking the nerves that received the ablation energy.

After the initial static and/or dynamic detection of bioelectric properties, the location of anatomical features can be used to determine where the treatment site(s) should be with respect to various anatomical structures for therapeutically effective neuromodulation of the targeted nerves. The bioelectric and other physiological properties described herein can be detected via electrodes (e.g., the electrodes (244, 336) of the end effector (214, 314)), and the electrode pairings on a device (e.g., end effector (214, 314)) can be selected to obtain the bioelectric data at specific zones or regions and at specific depths of the targeted regions. The specific properties detected at or surrounding target neuromodulation sites and associated methods for obtaining these properties are described below. These specific detection and mapping methods discussed below are described with reference to the system 100, although the methods can be implemented on other suitable systems and devices that provide for anatomical identification, anatomical mapping and/or neuromodulation therapy.

### Neural Identification and Mapping

In many neuromodulation procedures, it is beneficial to identify the portions of the nerves that fall within a zone and/or region of influence (referred to as the “interest zone”) of the energy delivered by a device 102, as well as the relative three-dimensional position of the neural tissue relative to the device 102. Characterizing the portions of the neural tissue within the interest zone and/or determining the relative positions of the neural tissue within the interest zone enables the clinician to (1) selectively activate target neural tissue over non-target structures (e.g., blood vessels), and (2) sub-select specific targeted neural tissue (e.g., parasympathetic nerves) over non-target neural tissue (e.g., sensory nerves, subgroups of neural tissue, neural tissue having certain compositions or morphologies). The target structures (e.g., parasympathetic nerves) and non-target structures (e.g., blood vessels, sensory nerves, etc.) can be identified based on the inherent signatures of specific structures, which are defined by the unique morphological compositions of the structures and the bioelectrical properties associated with these morphological compositions. For example, unique, discrete frequencies can be associated with morphological compositions and, therefore, be used to identify certain structures. The target and non-target structures can also be identified based on relative bioelectrical activation of the structures to sub-select specific neural structures. Further, target and non-target structures can be identified by the differing detected responses of the structures to a tailored injected stimuli. For example, the systems described herein can detect the magnitude of response of structures and the difference in the responses of anatomical structures with respect to differing stimuli (e.g., stimuli injected at different frequencies).

At least for purposes of this disclosure, a nerve can include the following portions that are defined based on their respective orientations relative to the interest zone: terminating neural tissue (e.g., terminating axonal structures), branching neural tissue (e.g., branching axonal structures), and traveling neural tissue (e.g., traveling axonal structures). For example, terminating neural tissue enter the zone but do not exit. As such, terminating neural tissue are terminal points for neuronal signaling and activation. Branching neural tissue are nerves that enter the interest zone and increase number of nerves exiting the interest zone. Branching neural tissue are typically associated with a reduction in relative geometry of nerve bundle. Traveling neural tissue are nerves that enter the interest zone and exit the zone with no substantially no change in geometry or numerical value.

The system 100 can be used to detect voltage, current, complex impedance, resistance, permittivity, and/or conductivity, which are tied to the compound action potentials of nerves, to determine and/or map the relative positions and proportionalities of nerves in the interest zone. Neuronal cross-sectional area (“CSA”) is expected to be due to the increase in axonic structures. Each axon is a standard size. Larger nerves (in cross-sectional dimension) have a larger number of axons than nerves having smaller cross-sectional dimensions. The compound action responses from the larger nerves, in both static and dynamic assessments, are greater than smaller nerves. This is at least in part because the compound action potential is the cumulative action response from each of the axons. When using static analysis, for example, the system 100 can directly measure and map impedance or resistance of nerves and, based on the determined impedance or resistance, determine the location of nerves and/or relative size of the nerves. In dynamic analysis, the system 100 can be used to apply a stimulus to the interest zone and detect the dynamic response of the neural tissue to the stimulus. Using this information, the system 100 can determine and/or map impedance or resistance in the interest zone to provide information related to the neural positions or relative nerve sizes. Neural impedance mapping can be illustrated by showing the varying complex impedance levels at a specific location at differing cross-sectional depths. In other embodiments, neural impedance or resistance can be mapped in a three-dimensional display.

Identifying the portions and/or relative positions of the nerves within the interest zone can inform and/or guide selection of one or more treatment parameters (e.g., electrode ablation patterns, electrode activation plans, etc.) of the system 100 for improving treatment efficiency and efficacy. For example, during neural monitoring and mapping, the system 100 can identify the directionality of the nerves based at least in part on the length of the neural structure extending along the interest zone, relative sizing of the neural tissue, and/or the direction of the action potentials. This information can then be used by the system 100 or the clinician to automatically or manually adjust treatment parameters (e.g., selective electrode activation, bipolar and/or multipolar activation, and/or electrode positioning) to target specific nerves or regions of nerves. For example, the system 100 can selectively activate specific electrodes (244, 336), electrode combinations (e.g., asymmetric or symmetric), and/or adjust the bi-polar or multi-polar electrode configuration. In some embodiments, the system 100 can adjust or select the waveform, phase angle, and/or other energy delivery parameters based on the nerve portion/position mapping and/or the nerve proportionality mapping. In some embodiments, structure and/or properties of the electrodes (244, 336) themselves (e.g., material, surface roughening, coatings, cross-sectional area, perimeter, penetrating, penetration depth, surface-mounted, etc.) may be selected based on the nerve portion and proportionality mapping.

In various embodiments, treatment parameters and/or energy delivery parameters can be adjusted to target on-axis or near axis traveling neural tissue and/or avoid the activation of traveling neural tissue that are at least generally perpendicular to the end effector (214, 314). Greater portions of the on-axis or near axis traveling neural tissue are exposed and susceptible to the neuromodulation energy provided by the end effector (214, 314) than a perpendicular traveling neural structure, which may only be exposed to therapeutic energy at a discrete cross-section. Therefore, the end effector (214, 314) is more likely to have a greater effect on the on-axis or near axis traveling neural tissue. The identification of the neural structure positions (e.g., via complex impedance or resistance mapping) can also allow targeted energy delivery to traveling neural tissue rather than branching neural tissue (typically downstream of the traveling neural tissue) because the traveling neural tissue are closer to the nerve origin and, therefore, more of the nerve is affected by therapeutic neuromodulation, thereby resulting in a more efficient treatment and/or a higher efficacy of treatment. Similarly, the identification of neural structure positions can be used to target traveling and branching neural tissue over terminal neural tissue. In some embodiments, the treatment parameters can be adjusted based on the detected neural positions to provide a selective regional effect. For example, a clinician can target downstream portions of the neural tissue if only wanting to influence partial effects on very specific anatomical structures or positions.

In various embodiments, neural locations and/or relative positions of nerves can be determined by detecting the nerve-firing voltage and/or current over time. An array of the electrodes (244, 336) can be positioned in contact with tissue at the interest zone, and the electrodes (244, 336) can measure the voltage and/or current associated with nerve-firing. This information can optionally be mapped (e.g., on a display 112) to identify the location of nerves in a hyper state (i.e., excessive parasympathetic tone). Rhinitis is at least in part the result of over-firing nerves because this hyper state drives the hyper-mucosal production and hyper-mucosal secretion. Therefore, detection of nerve firing rate via voltage and current measurements can be used to locate the portions of the interest region that include hyper-parasympathetic neural function (i.e., nerves in the diseased state). This allows the clinician to locate specific nerves (i.e., nerves with excessive parasympathetic tone) before neuromodulation therapy, rather than simply targeting all parasympathetic nerves (including non-diseased state parasympathetic nerves) to ensure that the correct tissue is treated during neuromodulation therapy. Further, nerve firing rate can be detected during or after neuromodulation therapy so that the clinician can monitor changes in nerve firing rate to validate treatment efficacy. For example, recording decreases or elimination of nerve firing rate after neuromodulation therapy can indicate that the therapy was effective in therapeutically treating the hyper/diseased nerves.

In various embodiments, the system 100 can detect neural activity using dynamic activation by injecting a stimulus signal (i.e., a signal that temporarily activates nerves) via one or more of the electrodes (244, 336) to induce an action potential, and other pairs of electrodes (244, 336) can detect bioelectric properties of the neural response. Detecting neural tissue using dynamic activation involves detecting the locations of action potentials within the interest zone by measuring the discharge rate in neurons and the associated processes. The ability to numerically measure, profile, map, and/or image fast neuronal depolarization for generating an accurate index of activity is a factor in measuring the rate of discharge in neurons and their processes. The action potential causes a rapid increase in the voltage across nerve fiber and the electrical impulse then spreads along the fiber. As an action potential occurs, the conductance of a neural cell membrane changes, becoming about 40 times larger than it is when the cell is at rest. During the action potential or neuronal depolarization, the membrane resistance diminishes by about 80 times, thereby allowing an applied current to enter the intracellular space as well. Over a population of neurons, this leads to a net decrease in the resistance during coherent neuronal activity, such as chronic para-sympathetic responses, as the intracellular space will provide additional conductive ions. The magnitude of such fast changes has been estimated to have local resistivity changes with recording near DC is 2.8-3.7% for peripheral nerve bundles.

Detecting neural tissue using dynamic activation includes detecting the locations of action potentials within the interest zone by measuring the discharge rate in neurons and the associated processes. The basis of each this discharge is the action potential, during which there is a depolarization of the neuronal membrane of up to 110 mV or more, lasting approximately 2 milliseconds, and due to the transfer of micromolar quantities of ions (e.g., sodium and potassium) across the cellular membrane. The complex impedance or resistance change due to the neuronal membrane falls from 1000 to 25 Ωcm. The introduction of a stimulus and subsequent measurement of the neural response can attenuate noise and improve signal to noise ratios to precisely focus on the response region to improve neural detection, measurement, and mapping.

In some embodiments, the difference in measurements of physiological parameters (e.g., complex impedance, resistance, voltage) over time, which can reduce errors, can be used to create a neural profiles, spectrums, or maps. For example, the sensitivity of the system 100 can be improved because this process provides repeated averaging to a stimulus. As a result, the mapping function outputs can be a unit-less ratio between the reference and test collated data at a single frequency and/or multiple frequencies and/or multiple amplitudes. Additional considerations may include multiple frequency evaluation methods that consequently expand the parameter assessments, such as resistivity, admittivity, center frequency, or ratio of extra- to intracellular resistivity.

In some embodiments, the system 100 may also be configured to indirectly measure the electrical activity of neural tissue to quantify the metabolic recovery processes that accompany action potential activity and act to restore ionic gradients to normal. These are related to an accumulation of ions in the extracellular space. The indirect measurement of electrical activity can be approximately a thousand times larger (in the order of millimolar), and thus are easier to measure and can enhance the accuracy of the measured electrical properties used to generate the neural maps.

The system 100 can perform dynamic neural detection by detecting nerve-firing voltage and/or current and, optionally, nerve firing rate over time, in response to an external stimulation of the nerves. For example, an array of the electrodes (244, 336) can be positioned in contact with tissue at the interest zone, one or more of the electrodes (244, 336) can be activated to inject a signal into the tissue that stimulates the nerves, and other electrodes (244, 336) of the electrode array can measure the neural voltage and/or current due to nerve firing in response to the stimulus. This information can optionally be mapped (e.g., on a display 112) to identify the location of nerves and, in certain embodiments, identify parasympathetic nerves in a hyper state (e.g., indicative of Rhinitis or other diseased state). The dynamic detection of neural activity (voltage, current, firing rate, etc.) can be performed before neuromodulation therapy to detect target nerve locations to select the target site and treatment parameters to ensure that the correct tissue is treated during neuromodulation therapy. Further, dynamic detection of neural activity can be performed during or after neuromodulation therapy to allow the clinician to monitor changes in neural activity to validate treatment efficacy. For example, recording decreases or elimination of neural activity after neuromodulation therapy can indicate that the therapy was effective in therapeutically treating the hyper/diseased nerves.

In some embodiments, a stimulating signal can be delivered to the vicinity of the targeted nerve via one or more penetrating electrodes (e.g., microneedles that penetrate tissue) associated with the end effector (214, 314) and/or a separate device. The stimulating signal generates an action potential, which causes smooth muscle cells or other cells to contract. The location and strength of this contraction can be detected via the penetrating electrode(s) and, thereby, indicate to the clinician the distance to the nerve and/or the location of the nerve relative to the stimulating needle electrode. In some embodiments, the stimulating electrical signal may have a voltage of typically 1-2 mA or greater and a pulse width of typically 100-200 microseconds or greater. Shorter pulses of stimulation result in better discrimination of the detected contraction, but may require more current. The greater the distance between the electrode and the targeted nerve, the more energy is required to stimulate. The stimulation and detection of contraction strength and/or location enables identification of how close or far the electrodes are from the nerve, and therefore can be used to localize the nerve spatially. In some embodiments, varying pulse widths may be used to measure the distance to the nerve. As the needle becomes closer to the nerve, the pulse duration required to elicit a response becomes less and less.

To localize nerves via muscle contraction detection, the system 100 can vary pulse-width or amplitude to vary the energy (Energy=pulse-width*amplitude) of the stimulus delivered to the tissue via the penetrating electrode(s). By varying the stimulus energy and monitoring muscle contraction via the penetrating electrodes and/or other type of sensor, the system 100 can estimate the distance to the nerve. If a large amount of energy is required to stimulate the nerve/contract the muscle, the stimulating/penetrating electrode is far from the nerve. As the stimulating/penetrating electrode, moves closer to the nerve, the amount of energy required to induce muscle contraction will drop. For example, an array of penetrating electrodes can be positioned in the tissue at the interest zone and one or more of the electrodes can be activated to apply stimulus at different energy levels until they induce muscle contraction. Using an iterative process, localize the nerve (e.g., via the mapping/evaluation/feedback algorithm 110).

In some embodiments, the system 100 can measure the muscular activation from the nerve stimulus (e.g., via the electrodes (244, 336)) to determine neural positioning for neural mapping, without the use of penetrating electrodes. In this embodiment, the treatment device targets the smooth muscle cells' varicosities surrounding the submucosal glands and the vascular supply, and then the compound muscle action potential. This can be used to summate voltage response from the individual muscle fiber action potentials. The shortest latency is the time from stimulus artifact to onset of the response. The corresponding amplitude is measured from baseline to negative peak and measured in millivolts (mV). Nerve latencies (mean±SD) in adults typically range about 2-6 milliseconds, and more typically from about 3.4±0.8 to about 4.0±0.5 milliseconds.

In some embodiments, the system 100 can record a neuromagnetic field outside of the nerves to determine the internal current of the nerves without physical disruption of the nerve membrane. Without being bound by theory, the contribution to the magnetic field from the current inside the membrane is two orders of magnitude larger than that from the external current, and that the contribution from current within the membrane is substantially negligible. Electrical stimulation of the nerve in tandem with measurements of the magnetic compound action fields (“CAFs”) can yield sequential positions of the current dipoles such that the location of the conduction change can be estimated (e.g., via the least-squares method). Visual representation (e.g., via the display 112) using magnetic contour maps can show normal or non-normal neural characteristics (e.g., normal can be equated with a characteristic quadrupolar pattern propagating along the nerve), and therefore indicate which nerves are in a diseases, hyperactive state and suitable targets for neuromodulation.

During magnetic field detection, an array of the electrodes (244, 336) can be positioned in contact with tissue at the interest zone and, optionally, one or more of the electrodes (244, 336) can be activated to inject an electrical stimulus into the tissue. As the nerves in the interest zone fire (either in response to a stimulus or in the absence of it), the nerve generates a magnetic field (e.g., similar to a current carrying wire), and therefore changing magnetic fields are indicative of the nerve nerve-firing rate. The changing magnetic field caused by neural firing can induce a current detected by nearby sensor wire (e.g., the sensor 314) and/or wires associated with the nearby electrodes (244, 336). By measuring this current, the magnetic field strength can be determined. The magnetic fields can optionally be mapped (e.g., on a display 112) to identify the location of nerves and select target nerves (nerves with excessive parasympathetic tone) before neuromodulation therapy to ensure that the desired nerves are treated during neuromodulation therapy. Further, the magnetic field information can be used during or after neuromodulation therapy so that the clinician can monitor changes in nerve firing rate to validate treatment efficacy.

In other embodiments, the neuromagnetic field is measured with a Hall Probe or other suitable device, which can be integrated into the end effector (214, 314) and/or part of a separate device delivered to the interest zone. Alternatively, rather than measuring the voltage in the second wire, the changing magnetic field can be measured in the original wire (i.e. the nerve) using a Hall probe. A current going through the Hall probe will be deflected in the semi-conductor. This will cause a voltage difference between the top and bottom portions, which can be measured. In some aspects of this embodiments, three orthogonal planes are utilized.

In some embodiments, the system 100 can be used to induce electromotive force (“EMF”) in a wire (i.e., a frequency-selective circuit, such as a tunable/LC circuit) that is tunable to resonant frequency of a nerve. In this embodiment, the nerve can be considered to be a current carrying wire, and the firing action potential is a changing voltage. This causes a changing current which, in turn, causes a changing magnetic flux (i.e., the magnetic field that is perpendicular to the wire). Under Faraday's Law of Induction/Faraday's Principle, the changing magnetic flux induces EMF (including a changing voltage) in a nearby sensor wire (e.g., integrated into the end effector (214, 314), the sensor 314, and/or other structure), and the changing voltage can be measured via the system 100.

In further embodiments, the sensor wire (e.g., the sensor 314) is an inductor and, therefore, provides an increase of the magnetic linkage between the nerve (i.e., first wire) and the sensor wire (i.e., second wire), with more turns for increasing effect. (e.g., V2,rms=V1,rms (N2/N1)). Due to the changing magnetic field, a voltage is induced in the sensor wire, and this voltage can be measured and used to estimate current changes in the nerve. Certain materials can be selected to enhance the efficiency of the EMF detection. For example, the sensor wire can include a soft iron core or other high permeability material for the inductor.

During induced EMF detection, the end effector (214, 314) and/or other device including a sensor wire is positioned in contact with tissue at the interest zone and, optionally, one or more of the electrodes (244, 336) can be activated to inject an electrical stimulus into the tissue. As the nerves in the interest zone fire (either in response to a stimulus or in the absence of it), the nerve generates a magnetic field (e.g., similar to a current carrying wire) that induces a current in the sensor wire (e.g., the sensor 314). This information can be used to determine neural location and/or map the nerves (e.g., on a display 112) to identify the location of nerves and select target nerves (nerves with excessive parasympathetic tone) before neuromodulation therapy to ensure that the desired nerves are treated during neuromodulation therapy. EMF information can also be used during or after neuromodulation therapy so that the clinician can monitor changes in nerve firing rate to validate treatment efficacy.

In some embodiments, the system 100 can detect magnetic fields and/or EMF generated at a selected frequency that corresponds to a particular type of nerve. The frequency and, by extension, the associated nerve type of the detected signal can be selected based on an external resonant circuit. Resonance occurs on the external circuit when it is matched to the frequency of the magnetic field of the particular nerve type and that nerve is firing. In manner, the system 100 can be used to locate a particular sub-group/type of nerves.

In some embodiments, the system 100 can include a variable capacitor frequency-selective circuit to identify the location and/or map specific nerves (e.g., parasympathetic nerve, sensory nerve, nerve fiber type, nerve subgroup, etc.). The variable capacitor frequency-selective circuit can be defined by the sensor 314 and/or other feature of the end effector (214, 314). Nerves have different resonant frequencies based on their function and structure. Accordingly, the system 100 can include a tunable LC circuit with a variable capacitor (C) and/or variable inductor (L) that can be selectively tuned to the resonant frequency of desired nerve types. This allows for the detection of neural activity only associated with the selected nerve type and its associated resonant frequency. Tuning can be achieved by moving the core in and out of the inductor. For example, tunable LC circuits can tune the inductor by: (i) changing the number of coils around the core; (ii) changing the cross-sectional area of the coils around the core; (iii) changing the length of the coil; and/or (iv) changing the permeability of the core material (e.g., changing from air to a core material). Systems including such a tunable LC circuit provide a high degree of dissemination and differentiation not only as to the activation of a nerve signal, but also with respect to the nerve type that is activated and the frequency at which the nerve is firing.

### Anatomical Mapping

In various embodiments, the system 100 is further configured to provide minimally-invasive anatomical mapping that uses focused energy current/voltage stimuli from a spatially localized source (e.g., the electrodes (244, 336)) to cause a change in the conductivity of the of the tissue at the interest zone and detect resultant biopotential and/or bioelectrical measurements (e.g., via the electrodes (244, 336)). The current density in the tissue changes in response to changes of voltage applied by the electrodes (244, 336), which creates a change in the electric current that can be measured with the end effector (214, 314) and/or other portions of the system 100. The results of the bioelectrical and/or biopotential measurements can be used to predict or estimate relative absorption profilometry to predict or estimate the tissue structures in the interest zone. More specifically, each cellular construct has unique conductivity and absorption profiles that can be indicative of a type of tissue or structure, such as bone, soft tissue, vessels, nerves, types of nerves, and/or certain neural tissue. For example, different frequencies decay differently through different types of tissue. Accordingly, by detecting the absorption current in a region, the system 100 can determine the underlying structure and, in some instances, to a sub-microscale, cellular level that allows for highly specialized target localization and mapping. This highly specific target identification and mapping enhances the efficacy and efficiency of neuromodulation therapy, while also enhancing the safety profile of the system 100 to reduce collateral effects on non-target structures.

To detect electrical and dielectric tissue properties (e.g., resistance, complex impedance, conductivity, and/or, permittivity as a function of frequency), the electrodes (244, 336) and/or another electrode array is placed on tissue at an interest region, and an internal or external source (e.g., the generator 106) applies stimuli (current/voltage) to the tissue. The electrical properties of the tissue between the source and the receiver electrodes (244, 336) are measured, as well as the current and/or voltage at the individual receiver electrodes (244, 336). These individual measurements can then be converted into an electrical map/image/profile of the tissue and visualized for the user on the display 112 to identify anatomical features of interest and, in certain embodiments, the location of firing nerves. For example, the anatomical mapping can be provided as a color-coded or gray-scale three-dimensional or two-dimensional map showing differing intensities of certain bioelectric properties (e.g., resistance, impedance, etc.), or the information can be processed to map the actual anatomical structures for the clinician. This information can also be used during neuromodulation therapy to monitor treatment progression with respect to the anatomy, and after neuromodulation therapy to validate successful treatment. In addition, the anatomical mapping provided by the bioelectrical and/or biopotential measurements can be used to track the changes to non-target tissue (e.g., vessels) due to neuromodulation therapy to avoid negative collateral effects. For example, a clinician can identify when the therapy begins to ligate a vessel and/or damage tissue, and modify the therapy to avoid bleeding, detrimental tissue ablation, and/or other negative collateral effects.

Furthermore, the threshold frequency of electric current used to identify specific targets can subsequently be used when applying therapeutic neuromodulation energy. For example, the neuromodulation energy can be applied at the specific threshold frequencies of electric current that are target neuronal-specific and differentiated from other non-targets (e.g., blood vessels, non-target nerves, etc.). Applying ablation energy at the target-specific frequency results in an electric field that creates ionic agitation in the target neural structure, which leads to differentials in osmotic potentials of the targeted neural tissue. These osmotic potential differentials cause dynamic changes in neuronal membronic potentials (resulting from the difference in intra-cellular and extra-cellular fluidic pressure) that lead to vacuolar degeneration of the targeted neural tissue and, eventually, necrosis. Using the highly targeted threshold neuromodulation energy to initiate the degeneration allows the system 100 to deliver therapeutic neuromodulation to the specific target, while surrounding blood vessels and other non-target structures are functionally maintained.

In some embodiments, the system 100 can further be configured to detect bioelectrical properties of tissue by non-invasively recording resistance changes during neuronal depolarization to map neural activity with electrical impedance, resistance, bio-impedance, conductivity, permittivity, and/or other bioelectrical measurements. Without being bound by theory, when a nerve depolarizes, the cell membrane resistance decreases (e.g., by approximately 80×) so that current will pass through open ion channels and into the intracellular space. Otherwise the current remains in the extracellular space. For non-invasive resistance measurements, tissue can be stimulated by applying a current of less than 100 Hz, such as applying a constant current square wave at 1 Hz with an amplitude less than 25% (e.g., 10%) of the threshold for stimulating neuronal activity, and thereby preventing or reducing the likelihood that the current does not cross into the intracellular space or stimulating at 2 Hz. In either case, the resistance and/or complex impedance is recorded by recording the voltage changes. A complex impedance or resistance map or profile of the area can then be generated.

For impedance/conductivity/permittivity detection, the electrodes (244, 336) and/or another electrode array are placed on tissue at an interest region, and an internal or external source (e.g., the generator 106) applies stimuli to the tissue, and the current and/or voltage at the individual receiver electrodes (244, 336) is measured. The stimuli can be applied at different frequencies to isolate different types of nerves. These individual measurements can then be converted into an electrical map/image/profile of the tissue and visualized for the user on the display 112 to identify anatomical features of interest. The neural mapping can also be used during neuromodulation therapy to select specific nerves for therapy, monitor treatment progression with respect to the nerves and other anatomy, and validate successful treatment.

In some embodiments of the neural and/or anatomical detection methods described above, the procedure can include comparing the mid-procedure physiological parameter(s) to the baseline physiological parameter(s) and/or other, previously-acquired mid-procedure physiological parameter(s) (within the same energy delivery phase). Such a comparison can be used to analyze state changes in the treated tissue. The mid-procedure physiological parameter(s) may also be compared to one or more predetermined thresholds, for example, to indicate when to stop delivering treatment energy. In some embodiments of the present technology, the measured baseline, mid-, and post-procedure parameters include a complex impedance. In some embodiments of the present technology, the post-procedure physiological parameters are measured after a pre-determined time period to allow the dissipation of the electric field effects (ionic agitation and/or thermal thresholds), thus facilitating accurate assessment of the treatment.

In some embodiments, the anatomical mapping methods described above can be used to differentiate the depth of soft tissues within the nasal mucosa. The depth of mucosa on the turbinates is relatively deep while the depth off the turbinate is relatively shallow and, therefore, identifying the tissue depth in the present technology also identifies positions within the nasal mucosa and where precisely to target. Further, by providing the micro-scale spatial impedance mapping of epithelial tissues as described above, the inherent unique signatures of stratified layers or cellular bodies can be used as identifying the region of interest. For example, different regions have larger or small populations of specific structures, such as submucosal glands, so target regions can be identified via the identification of these structures.

In some embodiments, the system 100 includes additional features that can be used to detect anatomical structures and map anatomical features. For example, the system 100 can include an ultrasound probe for identification of neural tissue and/or other anatomical structures. Higher frequency ultrasound provides higher resolution, but less depth of penetration. Accordingly, the frequency can be varied to achieve the appropriate depth and resolution for neural/anatomical localization. Functional identification may rely on the spatial pulse length (“SPL”) (wavelength multiplied by number of cycles in a pulse). Axial resolution (SPL/2) may also be determined to locate nerves.

In some embodiments, the system 100 can further be configured to emit stimuli with selective parameters that suppress rather than fully stimulate neural activity. For example, in embodiments where the strength-duration relationship for extracellular neural stimulation is selected and controlled, a state exists where the extracellular current can hyperpolarize cells, resulting in suppression rather than stimulation spiking behavior (i.e., a full action potential is not achieved). Both models of ion channels, Hodgkin-Huxley (HH) and Retinol Ganglion Cell (RGC), suggest that it is possible to hyperpolarize cells with appropriately designed burst extracellular stimuli, rather than extending the stimuli. This phenomenon could be used to suppress, rather than stimulate, neural activity during any of the embodiments of neural detection and/or modulation described herein.

As generally understood, the Hodgkin-Huxley (HH) model, or conductance-based model, is a mathematical model that describes how action potentials in neurons are initiated and propagated. It is a set of nonlinear differential equations that approximates the electrical characteristics of excitable cells such as neurons and cardiac myocytes and is a continuous-time dynamical system. A Hodgkin-Huxley type model represents the biophysical characteristic of cell membranes, as illustrated in the schematic diagram below:

The lipid bilayer is represented as a capacitance (Cₘ). Voltage-gated and leak ion channels are represented by nonlinear (gₙ) and linear (g_{L}) conductances, respectively. The electrochemical gradients driving the flow of ions are represented by batteries (E), and ion pumps and exchangers are represented by current sources (Iₚ).

A retinal ganglion cell (RGC) is a type of neuron located near the inner surface (the ganglion cell layer) of the retina of the eye. It receives visual information from photoreceptors via two intermediate neuron types: bipolar cells and retina amacrine cells. Retina amacrine cells, particularly narrow field cells, are important for creating functional subunits within the ganglion cell layer and making it so that ganglion cells can observe a small dot moving a small distance. Retinal ganglion cells collectively transmit image-forming and non-image forming visual information from the retina in the form of action potential to several regions in the thalamus, hypothalamus, and mesencephalon, or midbrain. The six types of retinal neurons are bipolar cells, ganglion cells, horizontal cells, retina amacrine cells, and rod and cone photoreceptors.

In various embodiments, the system 100 could apply the anatomical mapping techniques disclosed herein to locate or detect the targeted vasculature and surrounding anatomy before, during, and/or after treatment.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

1. A method for treating a condition, the method comprising:
providing a device comprising an end effector including a plurality of electrodes and a controller operably associated with the device;
positioning the end effector at a target site associated with a patient;
receiving, via the controller, data from the device associated with bioelectric properties of one or more tissues at the target site;
processing, via the controller, the data to identify a type of each of the one or more tissues at the target site and further identify a dielectric relaxation pattern for each of the one or more identified tissue types; and
determining, via the controller, an ablation pattern to be delivered by one or more of the plurality of electrodes of the end effector based on identified dielectric relaxation patterns, wherein ablation energy associated with the ablation pattern is at a level sufficient to ablate a targeted tissue and minimize and/or prevent collateral damage to surrounding or adjacent non-targeted tissue at the target site.

2. The method of claim 1, wherein a subset of the plurality of electrodes is configured to deliver non-therapeutic stimulating energy at a frequency/waveform to respective positions at the target site to thereby sense the bioelectric properties of the one or more tissues at the target site.

3. The method of claim 1, wherein the bioelectric properties comprise at least one of complex impedance, resistance, reactance, capacitance, inductance, permittivity, conductivity, dielectric properties, muscle or nerve firing voltage, muscle or nerve firing current, depolarization, hyperpolarization, magnetic field, and induced electromotive force.

4. The method of claim 3, wherein the dielectric properties comprise at least a complex, real and imaginary relative dielectric permittivity.

5. The method of claim 1, wherein the processing of the data, via the controller, comprises comparing the data received from the device with electric signature data associated with a plurality of known tissue types.

6. The method of claim 5, wherein the electric signature data comprises at least bioelectric properties and dielectric relaxation patterns of known tissue types.

7. The method of claim 6, wherein the dielectric relaxation patterns comprise at least one of a Maxwell-Wagner-Sillar (MWS) relaxation pattern, ionic relation pattern, and dielectric relaxation pattern.

8. The method of claim 5, wherein the comparison comprises correlating the data received from the device with electric signature data from a supervised and/or an unsupervised trained neural network.

9. The method of claim 1, wherein the ablation energy is tuned to a target frequency associated with relaxation patterns of the targeted tissue.

10. The method of claim 9, wherein the target frequency comprises a frequency at which the targeted tissue exhibits relaxation phenomena behavior and the non-targeted tissue does not exhibit relaxation phenomena behavior.

11. The method of claim 10, wherein delivery of the ablation energy, tuned to the target frequency, penetrates a membrane of one or more cells associated only with the targeted tissue.

12. The method of claim 1, wherein condition comprises a peripheral neurological condition.

13. The method of claim 12, wherein the peripheral neurological condition is associated with a nasal condition or a non-nasal condition of the patient.

14. The method of claim 13, wherein the non-nasal condition comprises atrial fibrillation (AF).

15. The method of claim 13, wherein the nasal condition comprises rhinosinusitis.

16. The method of claim 15, wherein the target site is within a sino-nasal cavity of the patient.

17. The method of claim 16, wherein delivery of the ablation energy results in disruption of multiple neural signals to, and/or result in local hypoxia of, mucus producing and/or mucosal engorgement elements within the sino-nasal cavity of the patient.

18. The method of claim 17, wherein the targeted tissue is proximate or inferior to a sphenopalatine foramen.

19. The method of claim 18, wherein delivery of the ablation energy results in therapeutic modulation of postganglionic parasympathetic nerves innervating nasal mucosa at foramina and or microforamina of a palatine bone of the patient.

20. The method of claim 19, wherein delivery of the ablation energy causes multiple points of interruption of neural branches extending through foramina and microforamina of palatine bone.

21. The method of claim 17, wherein delivery of the ablation energy causes thrombus formation within one or more blood vessels associated with mucus producing and/or mucosal engorgement elements within the nose.

22. The method of claim 21, wherein the resulting local hypoxia of the mucus producing and/or mucosal engorgement elements results in decreased mucosal engorgement to thereby increase volumetric flow through a nasal passage of the patient.

23-44. (canceled)
